(19) United States
(12) Patent Application Publication
(10) Pub. No.: US 2006/0002929 A1
(43) Pub. Date: Jan. 5, 2006

(54) Monoclonal antibodies

(76) Inventors: Sanjay D. Khare, Newbury Park, CA (US); Sirid-Aimee Kellermann, Menlo Park, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/086,289

(22) Filed: Mar. 23, 2005

Related U.S. Application Data
(60) Provisional application No. 60/555,396, filed on Mar. 23, 2004.

(51) Int. Cl. A61K 39/395; C07K 16/44

(52) U.S. Cl. 424/144.1; 530/387.3; 530/388.22

(57) ABSTRACT

Nucleotide sequences encoding, and amino acid sequences comprising, heavy and light chains of monoclonal antibodies.

This patent application claims the benefit of U.S. provisional application No. 60/555,396, filed on Mar. 23, 2004, which is incorporated by reference herein.

### FIELD

The present invention relates to antibodies reactive with OX40 ligand (OX40L), cells producing antibodies reactive with OX40L, pharmaceutical compositions comprising antibodies reactive with OX40L, methods using antibodies reactive with OX40L, and kits comprising antibodies reactive with OX40L.

### BACKGROUND

The interaction between OX40 and its ligand, OX40L, plays a role in the activation and expansion of antigen-activated CD4 T cells during an immune response. Initially, CD4 T cells are activated via the presentation of antigens bound to MHC-II and the T cell receptor (TCR). After antigen presentation, OX40 and OX40L cell surface expression is upregulated with OX40 expressed on the CD4 T cell surface and OX40L expressed on the antigen presenting cell (APC) surface. The combined signals of the antigen-TCR and OX40L-OX40 interactions facilitate CD4 T cell activation, expansion, migration, and cytokine production. See generally, e.g., Lane, P., J. Exp. Med. 191: 201-05 (2000).

OX40L is a member of the tumor necrosis factor (TNF) family of proteins. OX40L is typically expressed on APCs such as dendritic cells (DCs), macrophages, microglia, and B cells. OX40 is typically expressed in lymphoid tissue, e.g., in activated CD4 T cells. These OX40+T cells are preferentially found at sites of inflammation in the body. Likewise, in patients with an inflammatory condition, OX40L is typically expressed in tissues at the site of inflammation and not in healthy tissue. Investigators have demonstrated that reagents that inhibit the OX40L-OX40 interaction may be used to modulate T cell mediated experimental inflammatory diseases. See generally, e.g., Weinberg, A., Trends in Immunol. 23:102-09 (2002).

### SUMMARY OF THE INVENTION

In certain embodiments, an isolated polypeptide is provided comprising at least one complementarity determining region (CDR) selected from CDR1a, CDR2a, or CDR3a, wherein CDR1a comprises the amino acid sequence a b c d e, wherein amino acid a is selected from asparagine, threonine, phenylalanine, or serine; amino acid b is selected from alanine or tyrosine; amino acid c is selected from tryptophan, tyrosine, or glycine; amino acid d is selected from methionine or tryptophan; and amino acid e is selected from serine, asparagine, or histidine; wherein CDR2a comprises the amino acid sequence f g h i j k l m n o p q r s t, wherein amino acid f is selected from arginine or valine; amino acid g is isoleucine; amino acid h is selected from lysine, tyrosine, or tryptophan; amino acid i is selected from serine, isoleucine, tyrosine, threonine, or arginine; amino acid j is selected from lysine, serine, or aspartic acid; amino acid k is selected from threonine or glycine; amino acid l is selected from aspartic acid, serine, or glutamic acid; amino acid m is selected from glycine, threonine, or asparagine; amino acid n is selected from glycine, asparagine, lysine, or threonine; amino acid o is selected from threonine or tyrosine; amino acid p is selected from threonine, isoleucine, asparagine, or tyrosine; amino acid q is selected from aspartic acid, proline, or alanine; amino acid r is selected from tyrosine, serine, or aspartic acid; amino acid s is selected from glycine, alanine, leucine, or serine; and amino acid t is selected from alanine, lysine, or valine; wherein CDR3a comprises the amino acid sequence u v w x y z a′ b′ c′ d′ e′ f′, wherein amino acid u is selected from aspartic acid, glycine, methionine, or serine; amino acid v is selected from arginine, glycine, aspartic acid, tyrosine, or phenylalanine; amino acid w is selected from tyrosine, valine, glycine, or leucine; amino acid x is selected from phenylalanine, aspartic acid, tyrosine, or tryptophan; amino acid y is selected from phenylalanine, aspartic acid, tyrosine, or isoleucine; amino acid z is selected from glycine, tyrosine, proline, valine, or phenylalanine; amino acid a′ is selected from glutamic acid, serine, tyrosine, tryptophan, or alanine; amino acid b′ is selected from phenylalanine, glycine, tyrosine, threonine, or serine; amino acid c′ is selected from proline, tyrosine, serine, lysine, or glycine; amino acid d′ is selected from phenylalanine, tyrosine, or glycine; amino acid e′ is selected from aspartic acid, tyrosine, arginine, or histidine; and amino acid f′ is selected from tyrosine, valine, glycine, arginine, or threonine; and wherein the polypeptide, in association with an antibody light chain, is capable of binding OX40L.

In certain embodiments, an isolated polypeptide is provided comprising at least one complementarity determining region (CDR) selected from at least one of amino acids 50 to 54 of SEQ ID NO. 2, amino acids 69 to 87 of SEQ ID NO. 2, amino acids 120 to 135 of SEQ ID NO. 2, amino acids 50 to 54 of SEQ ID NO. 6, amino acids 69 to 84 of SEQ ID NO. 6, amino acids 117 to 134 of SEQ ID NO. 6, amino acids 50 to 54 of SEQ ID NO. 10, amino acids 69 to 85 of SEQ ID NO. 10, amino acids 118 to 135 of SEQ ID NO. 10, amino acids 50 to 54 of SEQ ID NO. 14, amino acids 69 to 84 of SEQ ID NO. 14, amino acids 117 to 131 of SEQ ID NO. 14, amino acids 50 to 54 of SEQ ID NO. 18, amino acids 69 to 87 of SEQ ID NO. 18, amino acids 120 to 133 of SEQ ID NO. 18, amino acids 50 to 54 of SEQ ID NO. 22, amino acids 69 to 87 of SEQ ID NO. 22, or amino acids 120 to 131 of SEQ ID NO. 22, wherein the polypeptide, in association with an antibody light chain, is capable of binding OX40L.

In certain embodiments, an isolated polypeptide is provided comprising at least one complementarity determining region (CDR) selected from CDR1b, CDR2b, or CDR3b wherein CDR1b comprises the amino acid sequence a1 b1 c1 d1 e1 f1 g1 h1 i1 j1 k1, wherein amino acid a1 is arginine; amino acid b1 is selected from alanine or serine; amino acid c1 is serine; amino acid d1 is glutamine; amino acid e1 is selected from glycine or serine; amino acid f1 is selected from isoleucine, valine, or leucine; amino acid g1 is selected from serine or valine; amino acid h1 is selected from asparagine, serine, or histidine; amino acid i1 is selected from histidine, asparagine, serine, or tyrosine; amino acid j1 is selected from leucine, tyrosine, or aspartic acid; and amino acid k1 is selected from valine, leucine, glycine, or asparagine; wherein CDR2b comprises the amino acid sequence l1 m1 n1 o1 p1 q1 r1, wherein amino acid l1 is selected from alanine, glycine, or lysine; amino acid m1 is selected from alanine or lysine; amino acid n1 is selected from serine or phenylalanine; amino acid o1 is selected from threonine, serine, or asparagine; amino acid p1 is selected from leucine or arginine; amino acid q1 is selected from glutamine, alanine, or phenylalanine; and amino acid r1 is selected from serine or threonine; wherein CDR3b comprises the amino acid sequence s1 t1 u1 v1 w1 x1 y1 z1 a1′, wherein amino acid s1 is selected from glutamine or methionine; and amino acid t1 is selected from lysine or glutamine; amino acid u1 is selected from tyrosine, alanine, serine, or phenylalanine; amino acid v1 is selected from asparagine, glycine, threonine, or tyrosine; amino acid w1 is selected from serine, glycine, or glutamine; amino acid x1 is selected from alanine, serine, isoleucine, or threonine; amino acid y1 is selected from proline or leucine; amino acid z1 is selected from leucine, tryptophan, or phenylalanine; and amino acid a1′ is threonine; and wherein the polypeptide, in association with an antibody heavy chain, is capable of binding OX40L.

In certain embodiments, an isolated polypeptide is provided comprising at least one complementarity determining region (CDR) selected from at least one of amino acids 46 to 56 of SEQ ID NO. 4, amino acids 72 to 78 of SEQ ID NO. 4, amino acids 111 to 119 of SEQ ID NO. 4, amino acids 46 to 56 of SEQ ID NO. 8, amino acids 72 to 78 of SEQ ID NO. 8, amino acids 111 to 119 of SEQ ID NO. 8, amino acids 44 to 59 of SEQ ID NO. 12, amino acids 75 to 81 of SEQ ID NO. 12, amino acids 114 to 122 of SEQ ID NO. 12, amino acids 44 to 55 of SEQ ID NO. 16, amino acids 71 to 77 of SEQ ID NO. 16, amino acids 110 to 118 of SEQ ID NO. 16, amino acids 46 to 56 of SEQ ID NO. 20, amino acids 72 to 78 of SEQ ID NO. 20, or amino acids 111 to 119 of SEQ ID NO. 20, wherein the polypeptide, in association with an antibody heavy chain, is capable of binding OX40L.

In certain embodiments, an isolated polynucleotide is provided comprising a sequence encoding a polypeptide comprising at least one complementarity determining region (CDR) selected from CDR1a, CDR2a, or CDR3a, wherein CDR1a comprises the amino acid sequence a b c d e, wherein amino acid a is selected from asparagine, threonine, phenylalanine, or serine; amino acid b is selected from alanine or tyrosine; amino acid c is selected from tryptophan, tyrosine, or glycine; amino acid d is selected from methionine or tryptophan; and amino acid e is selected from serine, asparagine, or histidine; wherein CDR2a comprises the amino acid sequence f g h i j k l m n o p q r s t, wherein amino acid f is selected from arginine or valine; amino acid g is isoleucine; amino acid h is selected from lysine, tyrosine, or tryptophan; amino acid i is selected from serine, isoleucine, tyrosine, threonine, or arginine; amino acid j is selected from lysine, serine, or aspartic acid; amino acid k is selected from threonine or glycine; amino acid l is selected from aspartic acid, serine, or glutamic acid; amino acid m is selected from glycine, threonine, or asparagine; amino acid n is selected from glycine, asparagine, lysine, or threonine; amino acid o is selected from threonine or tyrosine; amino acid p is selected from threonine, isoleucine, asparagine, or tyrosine; amino acid q is selected from aspartic acid, proline, or alanine; amino acid r is selected from tyrosine, serine, or aspartic acid; amino acid s is selected from glycine, alanine, leucine, or serine; and amino acid t is selected from alanine, lysine, or valine; wherein CDR3a comprises the amino acid sequence u v w x y z a′ b′ c′ d′ e′ f′, wherein amino acid u is selected from aspartic acid, glycine, methionine, or serine; amino acid v is selected from arginine, glycine, aspartic acid, tyrosine, or phenylalanine; amino acid w is selected from tyrosine, valine, glycine, or leucine; amino acid x is selected from phenylalanine, aspartic acid, tyrosine, or tryptophan; amino acid y is selected from phenylalanine, aspartic acid, tyrosine, or isoleucine; amino acid z is selected from glycine, tyrosine, proline, valine, or phenylalanine; amino acid a′ is selected from glutamic acid, serine, tyrosine, tryptophan, or alanine; amino acid b′ is selected from phenylalanine, glycine, tyrosine, threonine, or serine; amino acid c′ is selected from proline, tyrosine, serine, lysine, or glycine; amino acid d′ is selected from phenylalanine, tyrosine, or glycine; amino acid e′ is selected from aspartic acid, tyrosine, arginine, or histidine; and amino acid f′ is selected from tyrosine, valine, glycine, arginine, or threonine; and wherein the polypeptide, in association with an antibody light chain, is capable of binding OX40L.

In certain embodiments, an isolated polynucleotide is provided comprising a sequence encoding a polypeptide comprising at least one complementarity determining region (CDR) selected from CDR1b, CDR2b, or CDR3b wherein CDR1b comprises the amino acid sequence a1 b1 c1 d1 e1 f1 g1 h1 i1 j1 k1, wherein amino acid a1 is arginine; amino acid b1 is selected from alanine or serine; amino acid c1 is serine; amino acid d1 is glutamine; amino acid e1 is selected from glycine or serine; amino acid f1 is selected from isoleucine, valine, or leucine; amino acid g1 is selected from serine or valine; amino acid h1 is selected from asparagine, serine, or histidine; amino acid i1 is selected from histidine, asparagine, serine, or tyrosine; amino acid j1 is selected from leucine, tyrosine, or aspartic acid; and amino acid k1 is selected from valine, leucine, glycine, or asparagine; wherein CDR2b comprises the amino acid sequence 11 m1 n1 o1 p1 q1 r1, wherein amino acid l1 is selected from alanine, glycine, or lysine; amino acid m1 is selected from alanine or lysine; amino acid n1 is selected from serine or phenylalanine; amino acid o1 is selected from threonine, serine, or asparagine; amino acid p1 is selected from leucine or arginine; amino acid q1 is selected from glutamine, alanine, or phenylalanine; and amino acid r1 is selected from serine or threonine; wherein CDR3b comprises the amino acid sequence s1 t1 u1 v1 w1 x1 y1 z1 a1′, wherein amino acid s1 is selected from glutamine or methionine; and amino acid t1 is selected from lysine or glutamine; amino acid u1 is selected from tyrosine, alanine, serine, or phenylalanine; amino acid v1 is selected from asparagine, glycine, threonine, or tyrosine; amino acid w1 is selected from serine, glycine, or glutamine; amino acid x1 is selected from alanine, serine, isoleucine, or threonine; amino acid y1 is selected from proline or leucine; amino acid z1 is selected from leucine, tryptophan, or phenylalanine; and amino acid a1′ is threonine; and wherein the polypeptide, in association with an antibody heavy chain, is capable of binding OX40L.

In certain embodiments, an isolated anti-OX40L antibody is provided wherein the antibody comprises:
(i) a first polypeptide comprising at least one complementarity determining region (CDR) selected from CDR1a, CDR2a, or CDR3a;
   wherein CDR1a comprises the amino acid sequence a b c d e, wherein amino acid a is selected from asparagine, threonine, phenylalanine, or serine; amino acid b is selected from alanine or tyrosine; amino acid c is selected from tryptophan, tyrosine, or glycine; amino acid d is selected from methionine or tryptophan; and amino acid e is selected from serine, asparagine, or histidine;
   wherein CDR2a comprises the amino acid sequence f g h i j k l m n o p q r s t, wherein amino acid f is selected from arginine or valine; amino acid g is isoleucine; amino acid h is selected from lysine, tyrosine, or tryptophan; amino acid i is selected from serine, isoleucine, tyrosine, threonine, or arginine; amino acid j is selected from lysine, serine, or aspartic acid; amino acid k is selected from threonine or glycine; amino acid l is selected from aspartic acid, serine, or glutamic acid; amino acid m is selected from glycine, threonine, or asparagine; amino acid n is selected from glycine, asparagine, lysine, or threonine; amino acid o is selected from threonine or tyrosine; amino acid p is selected from threonine, isoleucine, asparagine, or tyrosine; amino acid q is selected from aspartic acid, proline, or alanine; amino acid r is selected from tyrosine, serine, or aspartic acid; amino acid s is selected from glycine, alanine, leucine, or serine; and amino acid t is selected from alanine, lysine, or valine;
   wherein CDR3a comprises the amino acid sequence u v w x y z a′ b′ c′ d′ e′ f′, wherein amino acid u is selected from aspartic acid, glycine, methionine, or serine; amino acid v is selected from arginine, glycine, aspartic acid, tyrosine, or phenylalanine; amino acid w is selected from tyrosine, valine, glycine, or leucine; amino acid x is selected from phenylalanine, aspartic acid, tyrosine, or tryptophan; amino acid y is selected from phenylalanine, aspartic acid, tyrosine, or isoleucine; amino acid z is selected from glycine, tyrosine, proline, valine, or phenylalanine; amino acid a′ is selected from glutamic acid, serine, tyrosine, tryptophan, or alanine; amino acid b′ is selected from phenylalanine, glycine, tyrosine, threonine, or serine; amino acid c′ is selected from proline, tyrosine, serine, lysine, or glycine; amino acid d′ is selected from phenylalanine, tyrosine, or glycine; amino acid e′ is selected from aspartic acid, tyrosine, arginine, or histidine; and amino acid f′ is selected from tyrosine, valine, glycine, arginine, or threonine; and wherein the first polypeptide, in association with an antibody light chain, is capable of binding OX40L; and
(ii) a second polypeptide comprising at least one complementarity determining region (CDR) selected from CDR1b, CDR2b, or CDR3b
   wherein CDR1b comprises the amino acid sequence a1 b1 c1 d1 e1 f1 g1 h1 i1 j1 k1, wherein amino acid a1 is arginine; amino acid b1 is selected from alanine or serine; amino acid c1 is serine; amino acid d1 is glutamine; amino acid e1 is selected from glycine or serine; amino acid f1 is selected from isoleucine, valine, or leucine; amino acid g1 is selected from serine or valine; amino acid h1 is selected from asparagine, serine, or histidine; amino acid i1 is selected from histidine, asparagine, serine, or tyrosine; amino acid j1 is selected from leucine, tyrosine, or aspartic acid; and amino acid k1 is selected from valine, leucine, glycine, or asparagine;
   wherein CDR2b comprises the amino acid sequence l1 m1 n1 o1 p1 q1 r1, wherein amino acid l1 is selected from alanine, glycine, or lysine; amino acid m1 is selected from alanine or lysine; amino acid n1 is selected from serine or phenylalanine; amino acid o1 is selected from threonine, serine, or asparagine; amino acid p1 is selected from leucine or arginine; amino acid q1 is selected from glutamine, alanine, or phenylalanine; and amino acid r1 is selected from serine or threonine;
   wherein CDR3b comprises the amino acid sequence s1 t1 u1 v1 w1 x1 y1 z1 a1′, wherein amino acid s1 is selected from glutamine or methionine; and amino acid t1 is selected from lysine or glutamine; amino acid u1 is selected from tyrosine, alanine, serine, or phenylalanine; amino acid v1 is selected from asparagine, glycine, threonine, or tyrosine; amino acid w1 is selected from serine, glycine, or glutamine; amino acid x1 is selected from alanine, serine, isoleucine, or threonine; amino acid y1 is selected from proline or leucine; amino acid z1 is selected from leucine, tryptophan, or phenylalanine; and amino acid a1′ is threonine; and
   wherein the second polypeptide, in association with an antibody heavy chain, is capable of binding OX40L.

In certain embodiments, an isolated anti-OX40L antibody is provided wherein the antibody comprises
a first polypeptide comprising complementarity determining regions (CDRs) as set forth in SEQ ID NO. 2 and a second polypeptide comprising CDRs as set forth in SEQ ID NO. 4;
a first polypeptide comprising CDRs as set forth in SEQ ID NO. 6 and a second polypeptide comprising CDRs as set forth in SEQ ID NO. 8;
a first polypeptide comprising CDRs as set forth in SEQ ID NO. 10 and a second polypeptide comprising CDRs as set forth in SEQ ID NO. 12;
a first polypeptide comprising CDRs as set forth in SEQ ID NO. 14 and a second polypeptide comprising CDRs as set forth in SEQ ID NO. 16; or
a first polypeptide comprising CDRs as set forth in SEQ ID NO. 18 and a second polypeptide comprising CDRs as set forth in SEQ ID NO. 20.

In certain embodiments, a method for detecting the presence or absence of OX40L in a sample is provided. In certain embodiments, such a method comprises (a) combining an anti-OX40L antibody and the sample; (b) separating antibodies bound to an antigen from unbound antibodies; and (c) detecting the presence or absence of antibodies bound to the antigen.

In certain embodiments, a method for isolating OX40L is provided. In certain embodiments, such a method comprises (a) attaching an anti-OX40L antibody to a substrate; (b) exposing a sample containing OX40L to the antibody of part (a); and (c) isolating OX40L.

In certain embodiments, a method for treating an inflammatory disease in a patient is provided. In certain embodiments, such a method comprises administering a therapeutically effective amount of an anti-OX40L antibody to the patient.

In certain embodiments, a method of making a polypeptide is provided. In certain embodiments, such a method comprises producing a polypeptide in a cell comprising an expression vector comprising a polynucleotide encoding a polypeptide comprising complementarity determining regions (CDRs) as set forth in SEQ ID NO. 2; SEQ ID NO. 6; SEQ ID NO. 10; SEQ ID NO. 14; SEQ ID NO. 18; or SEQ ID NO. 22, wherein the CDRs comprise an anti-OX40L antibody heavy chain variable region, in conditions suitable to express the polynucleotide contained therein to produce the polypeptide. In certain embodiments, such a method comprises producing a polypeptide in a cell comprising an expression vector comprising a polynucleotide encoding a polypeptide comprising complementarity determining regions (CDRs) as set forth in SEQ ID NO. 4; SEQ ID NO. 8; SEQ ID NO. 12; SEQ ID NO. 16; or SEQ ID NO. 20, wherein the CDRs comprise an anti-OX40L antibody light chain variable region, in conditions suitable to express the polynucleotide contained therein to produce the polypeptide.

In certain embodiments, a method of making an anti-OX40L antibody is provided. In certain embodiments, such a method comprises producing the antibody in a cell comprising an expression vector comprising a polynucleotide encoding a polypeptide comprising complementarity determining regions (CDRs) as set forth in SEQ ID NO. 2; SEQ ID NO. 6; SEQ ID NO. 10; SEQ ID NO. 14; SEQ ID NO. 18; or SEQ ID NO. 22, wherein the CDRs comprise an anti-OX40L antibody heavy chain variable region; and further comprising an expression vector comprising a polynucleotide encoding a polypeptide comprising complementarity determining regions (CDRs) as set forth in SEQ ID NO. 4; SEQ ID NO. 8; SEQ ID NO. 12; SEQ ID NO. 16; or SEQ ID NO. 20, wherein the CDRs comprise an anti-OX40L antibody light chain variable region, in conditions suitable to express the polynucleotides contained therein to produce the antibody.

In certain embodiments, a kit for detecting the presence or absence of OX40L in a sample is provided. In certain embodiments, such a kit comprises an anti-OX40L antibody and reagents for detecting the antibody.

In certain embodiments, a kit for isolating OX40L is provided. In certain embodiments, such a kit comprises an anti-OX40L antibody attached to a substrate and reagents for isolating OX40L.

In certain embodiments, a pharmaceutical composition comprising an anti-OX40L antibody and a pharmaceutically acceptable carrier is provided.

In certain embodiments, a isolated antibody is provided, wherein the antibody specifically binds to an epitope that is specifically bound by at least one of Ab A, Ab B, Ab C, Ab D, Ab E, Ab F, Ab G, Ab H, Ab I, or Ab J.

### BRIEF DESCRIPTION OF THE FIGURES

FIG. 1 shows the cDNA nucleotide sequence encoding the heavy chain of Ab A (SEQ ID NO. 1) and the amino acid sequence of the heavy chain of Ab A (SEQ ID NO. 2). Amino acids in the signal peptide and their corresponding encoding nucleotides are italicized. Amino acids in the framework regions of the variable region and their corresponding encoding nucleotides are in regular text. Amino acids in the CDRs of the variable region and their corresponding encoding nucleotides are in bold text. Amino acids in the constant region and their corresponding encoding nucleotides are underlined.

FIG. 2 shows the cDNA nucleotide sequence encoding the light chain of Ab A (SEQ ID NO. 3) and the amino acid sequence of the light chain of Ab A (SEQ ID NO. 4). Amino acids in the signal peptide and their corresponding encoding nucleotides are italicized. Amino acids in the framework regions of the variable region and their corresponding encoding nucleotides are in regular text. Amino acids in the CDRs of the variable region and their corresponding encoding nucleotides are in bold text. Amino acids in the constant region and their corresponding encoding nucleotides are underlined.

FIG. 3 shows the cDNA nucleotide sequence encoding the heavy chain of Ab B (SEQ ID NO. 5) and the amino acid sequence of the heavy chain of Ab B (SEQ ID NO. 6). Amino acids in the signal peptide and their corresponding encoding nucleotides are italicized. Amino acids in the framework regions of the variable region and their corresponding encoding nucleotides are in regular text. Amino acids in the CDRs of the variable region and their corresponding encoding nucleotides are in bold text. Amino acids in the constant region and their corresponding encoding nucleotides are underlined.

FIG. 4 shows the cDNA nucleotide sequence encoding the light chain of Ab B (SEQ ID NO. 7) and the amino acid sequence of the light chain of Ab B (SEQ ID NO. 8). Amino acids in the signal peptide and their corresponding encoding nucleotides are italicized. Amino acids in the framework regions of the variable region and their corresponding encoding nucleotides are in regular text. Amino acids in the CDRs of the variable region and their corresponding encoding nucleotides are in bold text. Amino acids in the constant region and their corresponding encoding nucleotides are underlined.

FIG. 5 shows the cDNA nucleotide sequence encoding the heavy chain of Ab C (SEQ ID NO. 9) and the amino acid sequence of the heavy chain of Ab C (SEQ ID NO. 10). Amino acids in the signal peptide and their corresponding encoding nucleotides are italicized. Amino acids in the framework regions of the variable region and their corresponding encoding nucleotides are in regular text. Amino acids in the CDRs of the variable region and their corresponding encoding nucleotides are in bold text. Amino acids in the constant region and their corresponding encoding nucleotides are underlined.

FIG. 6 shows the cDNA nucleotide sequence encoding the light chain of Ab C (SEQ ID NO. 11) and the amino acid sequence of the light chain of Ab C (SEQ ID NO. 12). Amino acids in the signal peptide and their corresponding encoding nucleotides are italicized. Amino acids in the framework regions of the variable region and their corresponding encoding nucleotides are in regular text. Amino acids in the CDRs of the variable region and their corresponding encoding nucleotides are in bold text. Amino acids in the constant region and their corresponding encoding nucleotides are underlined.

FIG. 7 shows the cDNA nucleotide sequence encoding the heavy chain of Ab D (SEQ ID NO. 13) and the amino acid sequence of the heavy chain of Ab D (SEQ ID NO. 14). Amino acids in the signal peptide and their corresponding encoding nucleotides are italicized. Amino acids in the framework regions of the variable region and their corresponding encoding nucleotides are in regular text. Amino acids in the CDRs of the variable region and their corresponding encoding nucleotides are in bold text. Amino acids in the constant region and their corresponding encoding nucleotides are underlined.

FIG. 8 shows the cDNA nucleotide sequence encoding the light chain of Ab D (SEQ ID NO. 15) and the amino acid sequence of the light chain of Ab D (SEQ ID NO. 16). Amino acids in the signal peptide and their corresponding encoding nucleotides are italicized. Amino acids in the framework regions of the variable region and their corresponding encoding nucleotides are in regular text. Amino acids in the CDRs of the variable region and their corresponding encoding nucleotides are in bold text. Amino acids in the constant region their and corresponding encoding nucleotides are underlined.

FIG. 9 shows the cDNA nucleotide sequence encoding the heavy chain of Abs E and F (SEQ ID NO. 17) and the amino acid sequence of the heavy chain of Abs E and F (SEQ ID NO. 18). Amino acids in the signal peptide and their corresponding encoding nucleotides are italicized. Amino acids in the framework regions of the variable region and their corresponding encoding nucleotides are in regular text. Amino acids in the CDRs of the variable region and their corresponding encoding nucleotides are in bold text. Amino acids in the constant region and their corresponding encoding nucleotides are underlined.

FIG. 10 shows the cDNA nucleotide sequence encoding the light chain of Abs E and F (SEQ ID NO. 19) and the amino acid sequence of the light chain of Abs E and F (SEQ ID NO. 20). Amino acids in the signal peptide and their corresponding encoding nucleotides are italicized. Amino acids in the framework regions of the variable region and their corresponding encoding nucleotides are in regular text. Amino acids in the CDRs of the variable region and their corresponding encoding nucleotides are in bold text. Amino acids in the constant region and their corresponding encoding nucleotides are underlined.

FIG. 11 shows the cDNA nucleotide sequence encoding the heavy chain of Ab G (SEQ ID NO. 21) and the amino acid sequence of the heavy chain of Ab G (SEQ ID NO. 22). Amino acids in the signal peptide and their corresponding encoding nucleotides are italicized. Amino acids in the framework regions of the variable region and their corresponding encoding nucleotides are in regular text. Amino acids in the CDRs of the variable region and their corresponding encoding nucleotides are in bold text. Amino acids in the constant region and their corresponding encoding nucleotides are underlined.

FIG. 12 shows the relatedness, as determined by Vector NTI software, of the amino acid sequences in certain different anti-OX40L human monoclonal antibodies. Numbers on the right side are the number of somatic mutations (amino acid differences from the closest germline sequence) in each V region.

FIG. 13 shows three representative graphs that compare the binding of Ab C, Ab D, and Ab F to human OX40L (right side up triangles), cynomolgus monkey OX40L (inverted triangles), human IL-1 receptor (squares), and mouse OX40L (diamonds), according to the work described in Example 2. MFI indicates mean fluorescence intensity.

FIG. 14 shows a representative graph of data from an equilibrium binding analysis. The MAbs were used at a fixed concentration of 0.2 nM. Results for Ab F (circles), Ab E (triangles), Ab C (inverted triangles), and Ab D (diamonds), according to the work described in Example 3, are shown.

FIG. 15 shows a representative graph of data from a competitive binding assay in which OX40L is expressed on HUVECs. The test antibodies used to compete for binding with the hFc-OX40R protein were Ab A (filled square), Ab E (filled inverted triangle), Ab I (filled circle), Ab B (open square), Ab H (open triangle), Ab C (open inverted triangle), Ab D (open diamond), Ab G (open circle), and Ab F (X symbol), according to the work described in Example 3.

FIG. 16 shows a representative graph of data from a whole blood assay measuring inhibition of IL-2 production. Blocking reagents were hFc-OX40R (X symbol), Ab E (upside down triangle), Ab D (right side up triangle), and Ab C (circles), according to the work described in Example 4.

FIG. 17 shows a representative graph of data from a co-stimulation assay measuring the ability of Ab C to block IL-2 production by human T cells, according to the work described in Example 4.

FIG. 18 shows a representative graph of data from a co-stimulation assay measuring the ability of Ab C to block IL-2 production by cynomolgous monkey T cells, according to the work described in Example 5. T cells from 4 cynomolgus monkey donors were tested. Co-stimulator hFc-OX40L was used at a final concentration of 2.5 μg/ml. Resulting ELISA OD values were converted into percentage of control values (POC) for graphical analysis.

FIG. 19 shows a representative graph of data from a co-stimulation assay measuring the ability of Ab C to block IL-2 production by cynomolgous monkey T cells, according to the work described in Example 5. T cells from the 4 cynomolgus monkey donors of FIG. 18 were tested. Co-stimulator hFc-OX40L was used at a final concentration of 1.25 μg/ml. Resulting ELISA OD values were converted into percentage of control values (POC) for graphical analysis.

FIG. 20 shows a representative graph of data from a PBMC assay measuring inhibition of T cell proliferation. Blocking reagents were Ab E (filled inverted triangle), Ab D (open triangle), Ab C (light filled circle), hFc-OX40R (X symbol), and IgG (dark filled circle), according to the work described in Example 6. The Y axis is the percent inhibition of ³H incorporation (in the presence of different concentrations of inhibitor antibodies, in relation to ³H incorporation with inducer alone (without IgG). The IgG control was also tested separately at different concentrations.

FIG. 21A shows a representative graph of data from a direct binding assay detecting the binding of Ab C or cFc-OX40R to OX40L expressed on CHO cells. Staining reagents are Ab C (triangle), human IgG (dark circle), and cFc-OX40R (light circle), according to the work described in Example 7. FIG. 21B shows a representative FACS analysis comparing the three staining groups with 5 μg/ml of staining reagent.

FIG. 22A shows a representative graph of data from a neutralization assay detecting the ability of Ab C obtained from various sources to neutralize the binding of cFc-OX40R to OX40L expressed on CHO cells. The neutralizing agents used are various lots of Ab C expressed in CHO cells (diamonds, squares, and triangles) and Ab C expressed from hybridoma cells (X symbol), according to the work described in Example 7. FIG. 22B shows the percent inhibition of cFc-OX40R binding, according to the work described in Example 7. The experiments of both figures use cFc-OX40R at 5 μg/ml.

FIG. 23 shows a FACS analysis of the neutralization activity of Ab C against cFc-OX40R at various concentrations of Ab C, according to the work described in Example 7.

### DETAILED DESCRIPTION OF CERTAIN PREFERRED EMBODIMENTS

The section headings used herein are for organizational purposes only and are not to be construed as limiting the subject matter described. All references or portions of references cited in this application are expressly incorporated by reference herein in their entirety for any purpose.

Definitions

Standard techniques may be used for recombinant DNA, oligonucleotide synthesis, and tissue culture and transfection (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is linked to a polynucleotide which it is not linked to in nature, or (3) does not occur in nature as part of a larger sequence.

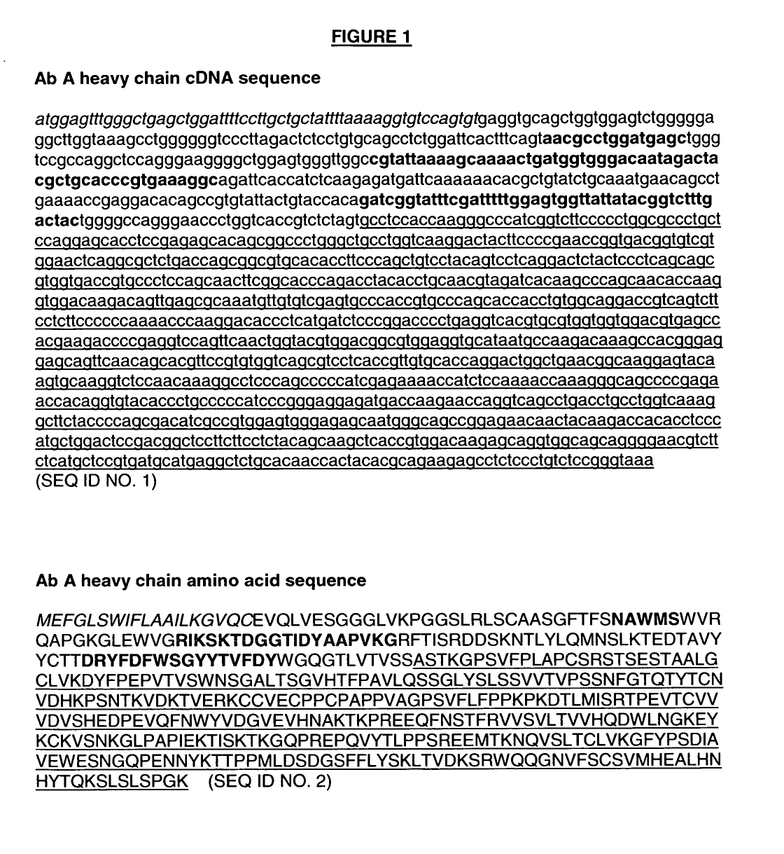

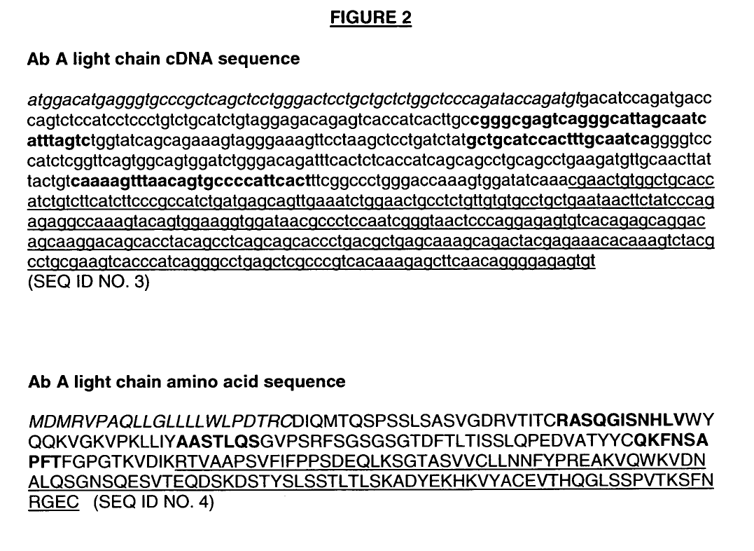

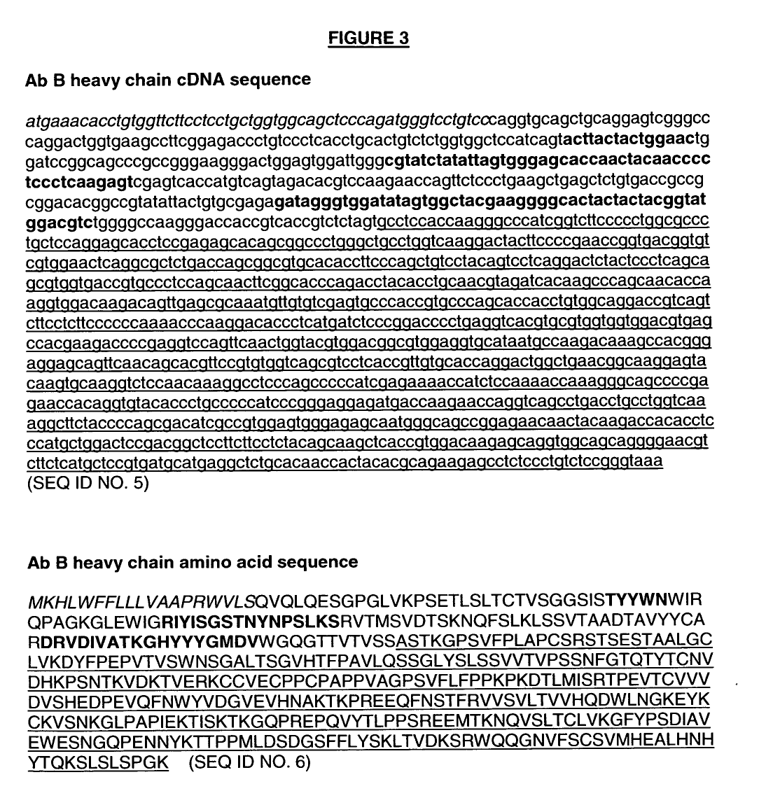

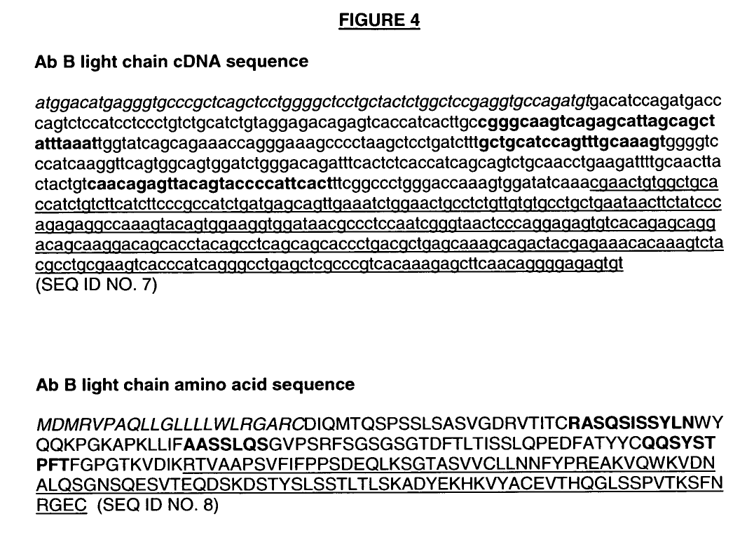

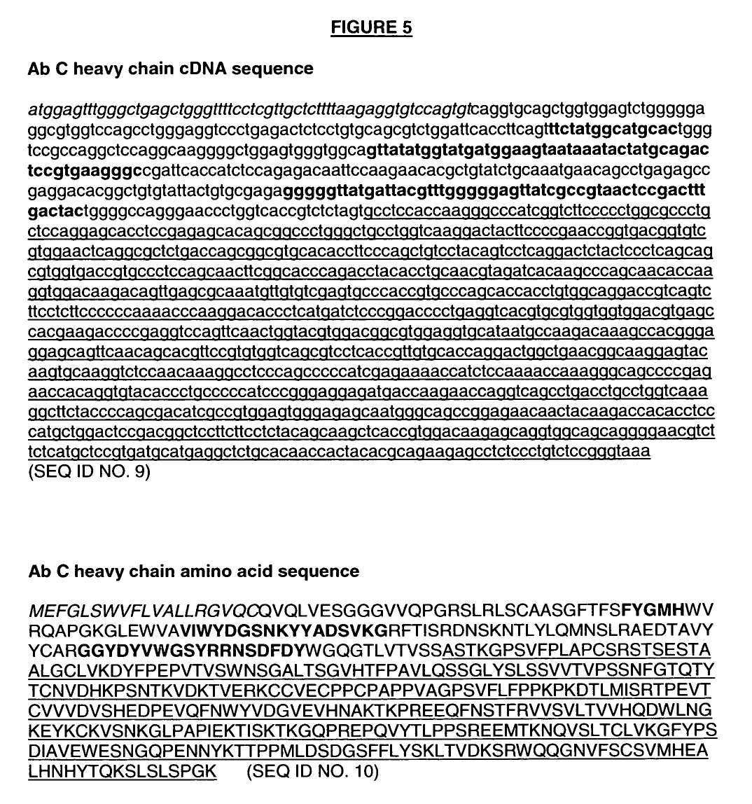

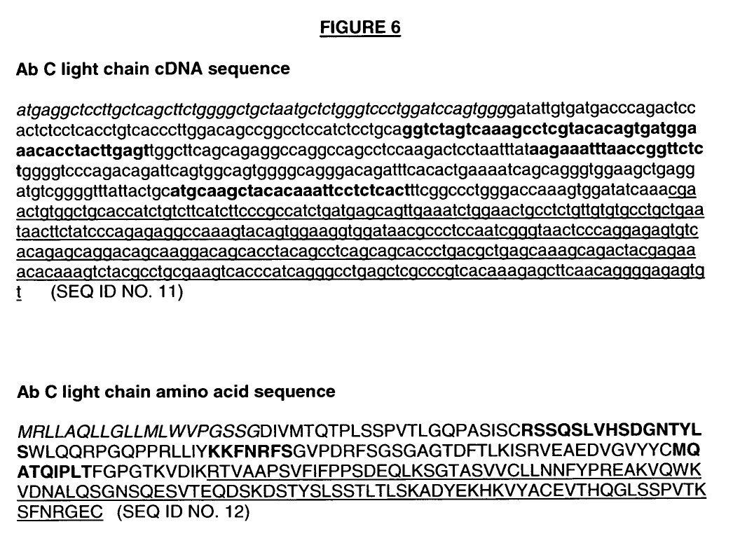

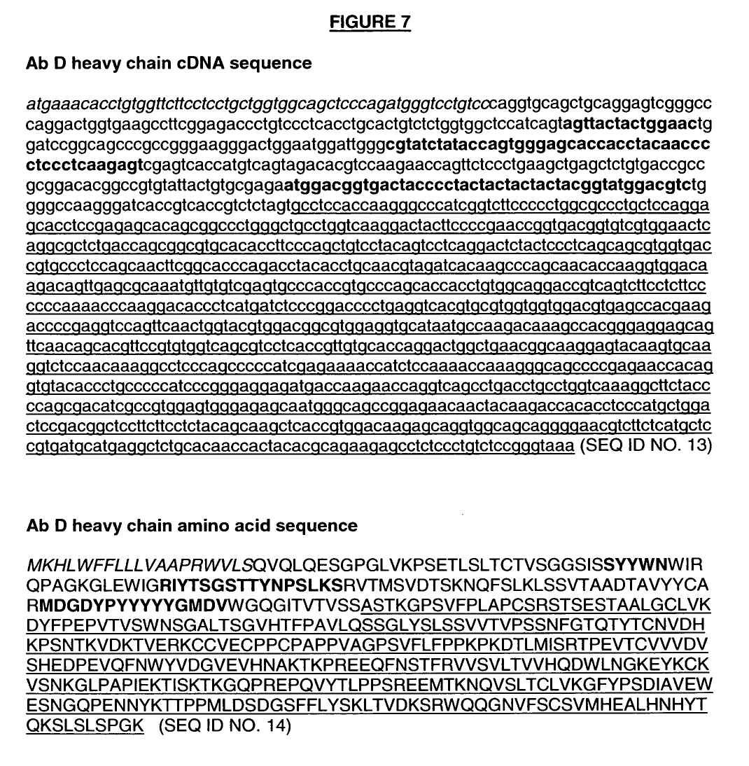

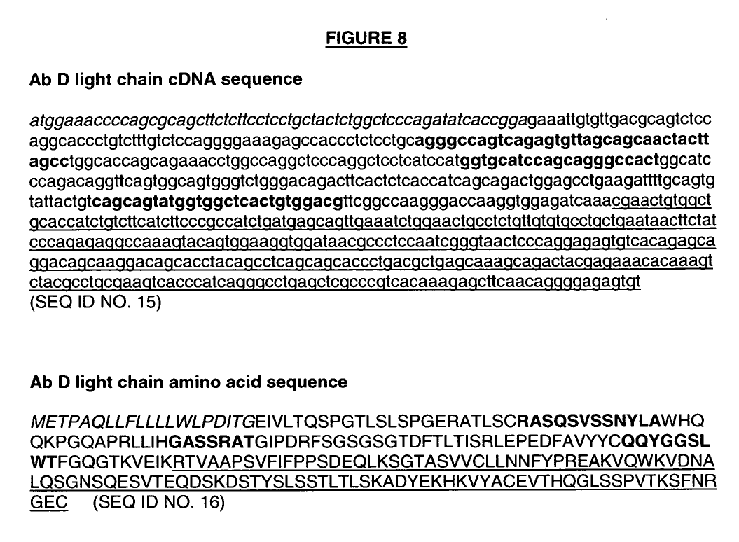

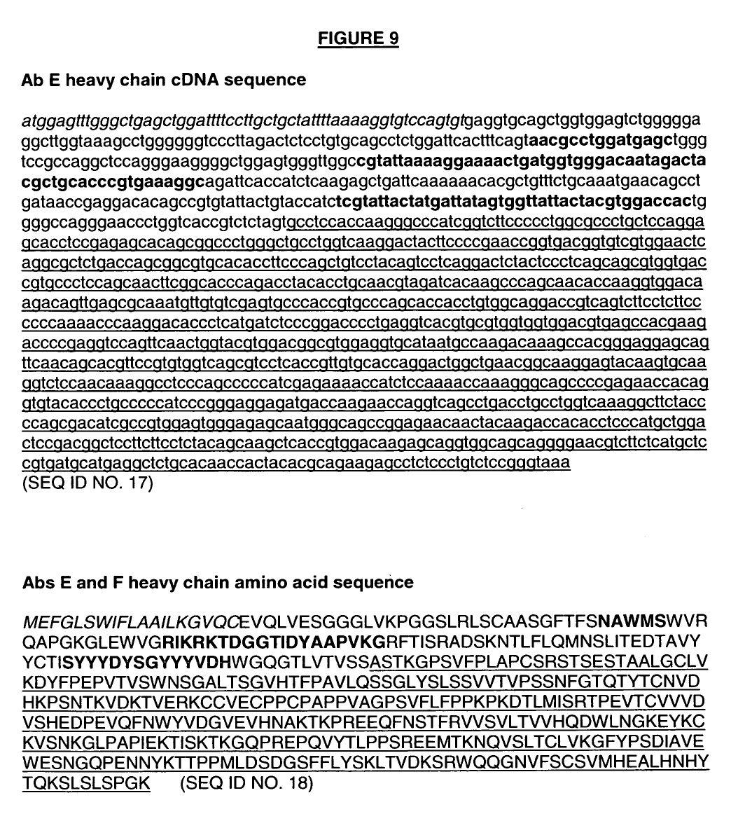

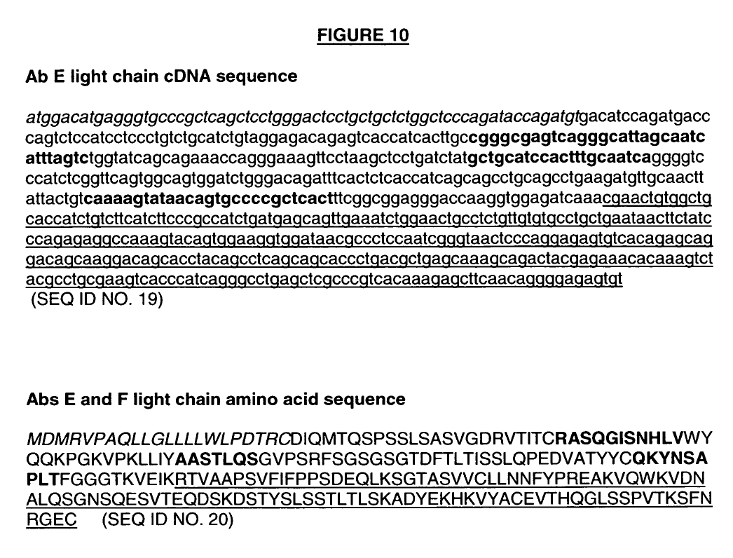

The term “polynucleotide” as referred to herein means a polymeric form of nucleotides of at least 10 bases in length. In certain embodiments, the bases may comprise at least one of ribonucleotides, deoxyribonucleotides, and a modified form of either type of nucleotide. The term includes single and double stranded forms of DNA. The term “polynucleotide” also encompasses sequences that comprise SEQ ID NOS: 1, 3, 5, 7, 9, 11, 13, 15, 17, 19, and 21. In certain embodiments, polynucleotides have nucleotide sequences that are about 90 percent, or about 95 percent, or about 96 percent, or about 97 percent, or about 98 percent, or about 99 percent identical to nucleotide sequences shown in FIGS. 1-11. In certain embodiments, polynucleotides complementary to specific polynucleotides that encode certain polypeptides described herein are provided.

In certain embodiments, a polynucleotide comprises a sequence encoding a polypeptide comprising at least one complementarity determining region (CDR) selected from CDR1a, CDR2a, or CDR3a, wherein CDR1a comprises the amino acid sequence a b c d e, wherein amino acid a is selected from asparagine, threonine, phenylalanine, or serine; amino acid b is selected from alanine or tyrosine; amino acid c is selected from tryptophan, tyrosine, or glycine; amino acid d is selected from methionine or tryptophan; and amino acid e is selected from serine, asparagine, or histidine; wherein CDR2a comprises the amino acid sequence f g h i j k l m n o p q r s t, wherein amino acid f is selected from arginine or valine; amino acid g is isoleucine; amino acid h is selected from lysine, tyrosine, or tryptophan; amino acid i is selected from serine, isoleucine, tyrosine, threonine, or arginine; amino acid j is selected from lysine, serine, or aspartic acid; amino acid k is selected from threonine or glycine; amino acid l is selected from aspartic acid, serine, or glutamic acid; amino acid m is selected from glycine, threonine, or asparagine; amino acid n is selected from glycine, asparagine, lysine, or threonine; amino acid o is selected from threonine or tyrosine; amino acid p is selected from threonine, isoleucine, asparagine, or tyrosine; amino acid q is selected from aspartic acid, proline, or alanine; amino acid r is selected from tyrosine, serine, or aspartic acid; amino acid s is selected from glycine, alanine, leucine, or serine; and amino acid t is selected from alanine, lysine, or valine; wherein CDR3a comprises the amino acid sequence u v w x y z a′ b′ c′ d′ e′ f′, wherein amino acid u is selected from aspartic acid, glycine, methionine, or serine; amino acid v is selected from arginine, glycine, aspartic acid, tyrosine, or phenylalanine; amino acid w is selected from tyrosine, valine, glycine, or leucine; amino acid x is selected from phenylalanine, aspartic acid, tyrosine, or tryptophan; amino acid y is selected from phenylalanine, aspartic acid, tyrosine, or isoleucine; amino acid z is selected from glycine, tyrosine, proline, valine, or phenylalanine; amino acid a′ is selected from glutamic acid, serine, tyrosine, tryptophan, or alanine; amino acid b′ is selected from phenylalanine, glycine, tyrosine, threonine, or serine; amino acid c′ is selected from proline, tyrosine, serine, lysine, or glycine; amino acid d′ is selected from phenylalanine, tyrosine, or glycine; amino acid e′ is selected from aspartic acid, tyrosine, arginine, or histidine; and amino acid f′ is selected from tyrosine, valine, glycine, arginine, or threonine; and wherein the polypeptide, in association with an antibody light chain, is capable of binding OX40L. In certain embodiments, a polynucleotide comprises a sequence encoding CDR2a comprising the amino acid sequence f g h i j k l m n o p q r s t g′ wherein f through t is an amino acid sequence as defined above and wherein amino acid g′ is selected from proline, lysine, or serine. In certain embodiments, a polynucleotide comprises a sequence encoding CDR2a comprising the amino acid sequence f g h i j k l m n o p q r s t g′ h′ wherein f through g′ is an amino acid sequence as defined above and wherein amino acid h′ is selected from valine or glycine. In certain embodiments, a polynucleotide comprises a sequence encoding CDR2a comprising the amino acid sequence f g h i j k l m n o p q r s t g′ h′ i′ wherein f through h′ is an amino acid sequence as defined above and wherein amino acid i′ is lysine. In certain embodiments, a polynucleotide comprises a sequence encoding CDR2a comprising the amino acid sequence f g h i j k l m n o p q r s t g′ h′ i′ j′ wherein f through i′ is an amino acid sequence as defined above and wherein amino acid j′ is glycine. In certain embodiments, a polynucleotide comprises a sequence encoding CDR3a comprising the amino acid sequence u v w x y z a′ b′ c′ d′ e′ f′ k′ wherein u through f′ is an amino acid sequence as defined above and wherein amino acid k′ is selected from aspartic acid, methionine, asparagine, tyrosine, or valine. In certain embodiments, a polynucleotide comprises a sequence encoding CDR3a comprising the amino acid sequence u v w x y z a′ b′ c′ d′ e′ f′ k′ l′ wherein u through k′ is an amino acid sequence as defined above and wherein amino acid l′ is selected from histidine, aspartic acid, serine, tyrosine, or phenylalanine. In certain embodiments, a polynucleotide comprises a sequence encoding CDR3a comprising the amino acid sequence u v w x y z a′ b′ c′ d′ e′ f′ k′ l′ m′ wherein u through l′ is an amino acid sequence as defined above and wherein amino acid m′ is selected from valine, aspartic acid, or glycine. In certain embodiments, a polynucleotide comprises a sequence encoding CDR3a comprising the amino acid sequence u v w x y z a′ b′ c′ d′ e′ f′ k′ l′ m′ n′ wherein u through m′ is an amino acid sequence as defined above and wherein amino acid n′ is selected from phenylalanine, methionine, or tyrosine. In certain embodiments, a polynucleotide comprises a sequence encoding CDR3a comprising the amino acid sequence u v w x y z a′ b′ c′ d′ e′ f′ k′ l′ m′ n′ o′ wherein u through n′ is an amino acid sequence as defined above and wherein amino acid o′ is aspartic acid. In certain embodiments, a polynucleotide comprises a sequence encoding CDR3a comprising the amino acid sequence u v w x y z a′ b′ c′ d′ e′ f′ k′ l′ m′ n′ o′ p′ wherein u through o′ is an amino acid sequence as defined above and wherein amino acid p′ is selected from valine or tyrosine.

In certain embodiments, a polynucleotide comprises a sequence encoding a polypeptide comprising at least two complementarity determining regions (CDR) selected from CDR1a, CDR2a, or CDR3a, wherein the polypeptide, in association with an antibody light chain, is capable of binding OX40L. In certain embodiments, a polynucleotide comprises a sequence encoding a polypeptide comprising CDR1a, CDR2a, and CDR3a, wherein the polypeptide, in association with an antibody light chain, is capable of binding OX40L.

In certain embodiments, a polynucleotide comprises a sequence encoding a polypeptide comprising an antibody heavy chain variable region. In certain embodiments, a polynucleotide comprises a sequence encoding a polypeptide comprising a human antibody heavy chain variable region. In certain embodiments, a polynucleotide comprises a sequence encoding a polypeptide comprising a heavy chain constant region. In certain embodiments, a polynucleotide comprises a sequence encoding a polypeptide comprising a human heavy chain constant region. In certain embodiments, a polynucleotide comprises a sequence encoding a polypeptide comprising an amino acid sequence as set forth in SEQ ID NO. 2; SEQ ID NO. 6; SEQ ID NO. 10; SEQ ID NO. 14; SEQ ID NO. 18; or SEQ ID NO. 22. In certain embodiments, a polynucleotide comprises a sequence encoding a polypeptide comprising a non-human heavy chain constant region. In certain embodiments, a polynucleotide comprises a sequence encoding a polypeptide comprising a heavy chain constant region of a species other than human.

In certain embodiments, a polynucleotide comprises a sequence encoding a polypeptide comprising at least one complementarity determining region (CDR) selected from at least one of amino acids 50 to 54 of SEQ ID NO. 2; amino acids 69 to 87 of SEQ ID NO. 2; amino acids 120 to 135 of SEQ ID NO. 2; amino acids 50 to 54 of SEQ ID NO. 6; amino acids 69 to 84 of SEQ ID NO. 6; amino acids 117 to 134 of SEQ ID NO. 6; amino acids 50 to 54 of SEQ ID NO. 10; amino acids 69 to 85 of SEQ ID NO. 10; amino acids 118 to 135 of SEQ ID NO. 10; amino acids 50 to 54 of SEQ ID NO. 14; amino acids 69 to 84 of SEQ ID NO. 14; amino acids 117 to 131 of SEQ ID NO. 14; amino acids 50 to 54 of SEQ ID NO. 18; amino acids 69 to 87 of SEQ ID NO. 18; amino acids 120 to 133 of SEQ ID NO. 18; amino acids 50 to 54 of SEQ ID NO. 22; amino acids 69 to 87 of SEQ ID NO. 22; or amino acids 120 to 131 of SEQ ID NO. 22, wherein the polypeptide, in association with an antibody light chain, is capable of binding OX40L. In certain embodiments, a polynucleotide comprises a sequence encoding a polypeptide comprising at least two of the CDRs of SEQ ID NOS. 2, 6, 10, 14, 18, or 22. In certain embodiments, a polynucleotide comprises a sequence encoding a polypeptide comprising three of the CDRs of SEQ ID NOS. 2, 6, 10, 14, 18, or 22.

In certain embodiments, a polynucleotide comprises a sequence encoding a polypeptide comprising amino acids 50 to 54 of SEQ ID NO. 2, amino acids 69 to 87 of SEQ ID NO. 2, and amino acids 120 to 135 of SEQ ID NO. 2. In certain embodiments, a polynucleotide comprises a sequence encoding a polypeptide comprising amino acids 50 to 54 of SEQ ID NO. 6, amino acids 69 to 84 of SEQ ID NO. 6, and amino acids 117 to 134 of SEQ ID NO. 6. In certain embodiments, a polynucleotide comprises a sequence encoding a polypeptide comprising amino acids 50 to 54 of SEQ ID NO. 10, amino acids 69 to 85 of SEQ ID NO. 10, and amino acids 118 to 135 of SEQ ID NO. 10. In certain embodiments, a polynucleotide comprises a sequence encoding a polypeptide comprising amino acids 50 to 54 of SEQ ID NO. 14, amino acids 69 to 84 of SEQ ID NO. 14, and amino acids 117 to 131 of SEQ ID NO. 14. In certain embodiments, a polynucleotide comprises a sequence encoding a polypeptide comprising amino acids 50 to 54 of SEQ ID NO. 18, amino acids 69 to 87 of SEQ ID NO. 18, and amino acids 120 to 133 of SEQ ID NO. 18. In certain embodiments, a polynucleotide comprises a sequence encoding a polypeptide comprising amino acids 50 to 54 of SEQ ID NO. 22, amino acids 69 to 87 of SEQ ID NO. 22, and amino acids 120 to 131 of SEQ ID NO. 22.

In certain embodiments, a polynucleotide comprises a sequence encoding a polypeptide comprising at least one complementarity determining region (CDR) selected from CDR1b, CDR2b, or CDR3b, wherein CDR1b comprises the amino acid sequence a1 b1 c1 d1 e1 f1 g1 h1 i1 j1 k1, wherein amino acid a1 is arginine; amino acid b1 is selected from alanine or serine; amino acid c1 is serine; amino acid d1 is glutamine; amino acid e1 is selected from glycine or serine; amino acid f1 is selected from isoleucine, valine, or leucine; amino acid g1 is selected from serine or valine; amino acid h1 is selected from asparagine, serine, or histidine; amino acid i1 is selected from histidine, asparagine, serine, or tyrosine; amino acid j1 is selected from leucine, tyrosine, or aspartic acid; and amino acid k1 is selected from valine, leucine, glycine, or asparagine; wherein CDR2b comprises the amino acid sequence l1 m1 n1 o1 p1 q1 r1, wherein amino acid l1 is selected from alanine, glycine, or lysine; amino acid m1 is selected from alanine or lysine; amino acid n1 is selected from serine or phenylalanine; amino acid o1 is selected from threonine, serine, or asparagine; amino acid p1 is selected from leucine or arginine; amino acid q1 is selected from glutamine, alanine, or phenylalanine; and amino acid r1 is selected from serine or threonine; wherein CDR3b comprises the amino acid sequence s1 t1 u1 v1 w1 x1 y1 z1 a1′, wherein amino acid s1 is selected from glutamine or methionine; and amino acid t1 is selected from lysine or glutamine; amino acid u1 is selected from tyrosine, alanine, serine, or phenylalanine; amino acid v1 is selected from asparagine, glycine, threonine, or tyrosine; amino acid w1 is selected from serine, glycine, or glutamine; amino acid x1 is selected from alanine, serine, isoleucine, or threonine; amino acid y1 is selected from proline or leucine; amino acid z1 is selected from leucine, tryptophan, or phenylalanine; and amino acid a1′ is threonine; and wherein the polypeptide, in association with an antibody heavy chain, is capable of binding OX40L. In certain embodiments, a polynucleotide comprises a sequence encoding CDR1b comprising the amino acid sequence a1 b1 c1 d1 e1 f1 g1 h1 i1 j1 k1 b1′ wherein a1 through k1 is an amino acid sequence as defined above and wherein amino acid b1′ is selected from asparagine or alanine. In certain embodiments, a polynucleotide comprises a sequence encoding CDR1b comprising the amino acid sequence a1 b1 c1 d1 e1 f1 g1 h1 i1 j1 k1 b1′ c1′ wherein a1 through b1′ is an amino acid sequence as defined above and wherein amino acid c1′ is threonine. In certain embodiments, a polynucleotide comprises a sequence encoding CDR1b comprising the amino acid sequence a1 b1 c1 d1 e1 f1 g1 h1 i1 j1 k1 b1′ c1′ d1′ wherein a1 through c1′ is an amino acid sequence as defined above and wherein amino acid d1′ is tyrosine. In certain embodiments, a polynucleotide comprises a sequence encoding CDR1b comprising the amino acid sequence a1 b1 c1 d1 e1 f1 g1 h1 i1 j1 k1 b1′ c1′ d1′ e1′ wherein a1 through d1′ is an amino acid sequence as defined above and wherein amino acid e1′ is leucine. In certain embodiments, a polynucleotide comprises a sequence encoding CDR1b comprising the amino acid sequence a1 b1 c1 d1 e1 f1 g1 h1 i1 j1 k1 b1′ c1′ d1′ e1′ f1′ wherein a1 through e1′ is an amino acid sequence as defined above and wherein amino acid f1′ is serine.

In certain embodiments, a polynucleotide comprises a sequence encoding a polypeptide comprising at least two complementarity determining regions (CDR) selected from CDR1b, CDR2b, or CDR3b, wherein the polypeptide, in association with an antibody heavy chain, is capable of binding OX40L. In certain embodiments, a polynucleotide comprises a sequence encoding a polypeptide comprising CDR1b, CDR2b, and CDR3b, wherein the polypeptide, in association with an antibody heavy chain, is capable of binding OX40L.

In certain embodiments, a polynucleotide comprises a sequence encoding a polypeptide comprising an antibody light chain variable region. In certain embodiments, a polynucleotide comprises a sequence encoding a polypeptide comprising a human antibody light chain variable region. In certain embodiments, a polynucleotide comprises a sequence encoding a polypeptide comprising a light chain constant region. In certain embodiments, a polynucleotide comprises a sequence encoding a polypeptide comprising a human light chain constant region. In certain embodiments, a polynucleotide comprises a sequence encoding a polypeptide comprising an amino acid sequence as set forth in SEQ ID NO. 4; SEQ ID NO. 8; SEQ ID NO. 12; SEQ ID NO. 16; or SEQ ID NO. 20. In certain embodiments, a polynucleotide comprises a sequence encoding a polypeptide comprising a non-human light chain constant region. In certain embodiments, a polynucleotide comprises a sequence encoding a polypeptide comprising a light chain constant region of a species other than human.

In certain embodiments, a polynucleotide comprises a sequence encoding a polypeptide comprising at least one complementarity determining region (CDR) selected from at least one of amino acids 46 to 56 of SEQ ID NO. 4; amino acids 72 to 78 of SEQ ID NO. 4; amino acids 111 to 119 of SEQ ID NO. 4; amino acids 46 to 56 of SEQ ID NO. 8; amino acids 72 to 78 of SEQ ID NO. 8; amino acids 111 to 119 of SEQ ID NO. 8; amino acids 44 to 59 of SEQ ID NO. 12; amino acids 75 to 81 of SEQ ID NO. 12; amino acids 114 to 122 of SEQ ID NO. 12; amino acids 44 to 55 of SEQ ID NO. 16; amino acids 71 to 77 of SEQ ID NO. 16; amino acids 110 to 118 of SEQ ID NO. 16; amino acids 46 to 56 of SEQ ID NO. 20; amino acids 72 to 78 of SEQ ID NO. 20; or amino acids 111 to 119 of SEQ ID NO. 20, wherein the polypeptide, in association with an antibody heavy chain, is capable of binding OX40L. In certain embodiments, a polynucleotide comprises a sequence encoding a polypeptide comprising at least two of the CDRs of SEQ ID NOS. 4, 8, 12, 16, or 20. In certain embodiments, a polynucleotide comprises a sequence encoding a polypeptide comprising three of the CDRs of SEQ ID NOS. 4, 8, 12, 16, or 20.

In certain embodiments, a polynucleotide comprises a sequence encoding a polypeptide comprising amino acids 46 to 56 of SEQ ID NO. 4, amino acids 72 to 78 of SEQ ID NO. 4, and amino acids 111 to 119 of SEQ ID NO. 4. In certain embodiments, a polynucleotide comprises a sequence encoding a polypeptide comprising amino acids 46 to 56 of SEQ ID NO. 8, amino acids 72 to 78 of SEQ ID NO. 8, and amino acids 111 to 119 of SEQ ID NO. 8. In certain embodiments, a polynucleotide comprises a sequence encoding a polypeptide comprising amino acids 44 to 59 of SEQ ID NO. 12, amino acids 75 to 81 of SEQ ID NO. 12, and amino acids 114 to 122 of SEQ ID NO. 12. In certain embodiments, a polynucleotide comprises a sequence encoding a polypeptide comprising amino acids 44 to 55 of SEQ ID NO. 16, amino acids 71 to 77 of SEQ ID NO. 16, and amino acids 110 to 118 of SEQ ID NO. 16. In certain embodiments, a polynucleotide comprises a sequence encoding a polypeptide comprising amino acids 46 to 56 of SEQ ID NO. 20, amino acids 72 to 78 of SEQ ID NO. 20, and amino acids 111 to 119 of SEQ ID NO. 20.

In certain embodiments, this application discusses certain polynucleotides encoding antibody heavy and light chains. In certain embodiments, this application discusses certain polynucleotides encoding an antibody heavy chain variable region. In certain embodiments, this application discusses certain polynucleotides encoding a human antibody heavy chain variable region. In certain embodiments, this application discusses certain polynucleotides encoding antibody light chain variable regions. In certain embodiments, this application discusses certain polynucleotides encoding a human antibody light chain variable region. In certain embodiments, this application discusses certain polynucleotides encoding an antibody heavy chain constant region. In certain embodiments, this application discusses certain polynucleotides encoding a human antibody heavy chain constant region. In certain embodiments, this application discusses certain polynucleotides encoding an antibody heavy chain constant region of a species other than human. In certain embodiments, this application discusses certain polynucleotides encoding antibody light chain constant regions. In certain embodiments, this application discusses certain polynucleotides encoding a human antibody light chain constant region. In certain embodiments, this application discusses certain polynucleotides encoding an antibody light chain constant region of a species other than human. In certain embodiments, this application discusses certain polynucleotides encoding a single-chain antibody.

In certain embodiments, these antibody heavy and light chain polynucleotides and polypeptides are human antibody heavy and light chain polynucleotides and polypeptides. In certain embodiments a polynucleotide comprises a nucleotide sequence as set forth in SEQ ID NOS. 1, 3, 5, 7, 9, 11, 13, 15, 17, 19, or 21, and sequences that have deletions, additions, and/or substitutions of one or more nucleotides of those sequences. In certain embodiments, a polynucleotide comprises a nucleotide sequence encoding an amino acid sequence comprising an amino acid sequence as set forth in SEQ ID NOS: 2, 4, 6, 8, 10, 12, 14, 16, 18, 20, or 22. In certain embodiments, variable region sequences comprising complementarity determining regions (CDRs), e.g., CDR1 through CDR3, are provided. In certain embodiments, variable region polynucleotides and polypeptides are human variable region polynucleotides and polypeptides.

The term “oligonucleotide” referred to herein includes naturally occurring and/or modified nucleotides linked together by naturally occurring, and/or non-naturally occurring oligonucleotide linkages. Oligonucleotides are a polynucleotide subset generally comprising a length of 200 bases or fewer. In certain embodiments, oligonucleotides are 10 to 60 bases in length. In certain embodiments, oligonucleotides are 12, 13, 14, 15, 16, 17, 18, 19, or 20 to 40 bases in length. Oligonucleotides may be single stranded or double stranded, e.g. for use in the construction of a gene mutant. Oligonucleotides of the invention may be sense or antisense oligonucleotides.

The term “naturally occurring nucleotides” includes deoxyribonucleotides and ribonucleotides. Deoxyribonucleotides include, but are not limited to, adenosine, guanine, cytosine, and thymidine. Ribonucleotides include, but are not limited to, adenosine, cytosine, thymidine, and uricil. The term “modified nucleotides” includes nucleotides with modified or substituted sugar groups and the like. The term “oligonucleotide linkages” includes oligonucleotides linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). In certain instances, an oligonucleotide can include a label for detection.

The term “isolated polypeptide” referred to herein means a polypeptide encoded by cDNA, recombinant RNA, or synthetic origin or some combination thereof, which (1) is free of at least some proteins with which it would normally be found, (2) is essentially free of other proteins from the same source, e.g., from the same species, (3) is expressed by a cell from a different species, or (4) does not occur in nature.

The term “polypeptide” is used herein as a generic term to refer to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than those normally encoded by a codon.

Polypeptides include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. Such modifications may be present to the same or varying degrees at several sites in a given polypeptide. Also, in certain embodiments, a given polypeptide may contain many types of modifications such as deletions, additions, and/or substitutions of one or more amino acids of a native sequence. In certain embodiments, polypeptides may be branched as a result of ubiquitination, and, in certain embodiments, they may be cyclic, with or without branching. Cyclic, branched and branched cyclic polypeptides may result from post-translation natural processes or may be made by synthetic methods. Modifications include, but are not limited to, acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. The term “polypeptide” also encompasses sequences that comprise the amino acid sequences of the heavy chain and/or light chain of Ab A, Ab B, Ab C, Ab D, Ab E, Ab F, Ab G, Ab H, Ab I, or Ab J as described below (see SEQ ID NOS: 2, 4, 6, 8, 10, 12, 14, 16, 18, 20, and 22 for certain such sequences), and sequences that have deletions, additions, and/or substitutions of one or more amino acids of those sequences. In certain embodiments, certain polypeptide sequences comprise at least one complementarity determining region (CDR).

In certain embodiments, a polypeptide comprises at least one complementarity determining region (CDR) selected from CDR1a, CDR2a, or CDR3a wherein CDR1a comprises the amino acid sequence a b c d e, wherein amino acid a is selected from asparagine, threonine, phenylalanine, or serine; amino acid b is selected from alanine or tyrosine; amino acid c is selected from tryptophan, tyrosine, or glycine; amino acid d is selected from methionine or tryptophan; and amino acid e is selected from serine, asparagine, or histidine; wherein CDR2a comprises the amino acid sequence f g h i j k l m n o p q r s t, wherein amino acid f is selected from arginine or valine; amino acid g is isoleucine; amino acid h is selected from lysine, tyrosine, or tryptophan; amino acid i is selected from serine, isoleucine, tyrosine, threonine, or arginine; amino acid j is selected from lysine, serine, or aspartic acid; amino acid k is selected from threonine or glycine; amino acid l is selected from aspartic acid, serine, or glutamic acid; amino acid m is selected from glycine, threonine, or asparagine; amino acid n is selected from glycine, asparagine, lysine, or threonine; amino acid o is selected from threonine or tyrosine; amino acid p is selected from threonine, isoleucine, asparagine, or tyrosine; amino acid q is selected from aspartic acid, proline, or alanine; amino acid r is selected from tyrosine, serine, or aspartic acid; amino acid s is selected from glycine, alanine, leucine, or serine; and amino acid t is selected from alanine, lysine, or valine; wherein CDR3a comprises the amino acid sequence u v w x y z a′ b′ c′ d′ e′ f′, wherein amino acid u is selected from aspartic acid, glycine, methionine, or serine; amino acid v is selected from arginine, glycine, aspartic acid, tyrosine, or phenylalanine; amino acid w is selected from tyrosine, valine, glycine, or leucine; amino acid x is selected from phenylalanine, aspartic acid, tyrosine, or tryptophan; amino acid y is selected from phenylalanine, aspartic acid, tyrosine, or isoleucine; amino acid z is selected from glycine, tyrosine, proline, valine, or phenylalanine; amino acid a′ is selected from glutamic acid, serine, tyrosine, tryptophan, or alanine; amino acid b′ is selected from phenylalanine, glycine, tyrosine, threonine, or serine; amino acid c′ is selected from proline, tyrosine, serine, lysine, or glycine; amino acid d′ is selected from phenylalanine, tyrosine, or glycine; amino acid e′ is selected from aspartic acid, tyrosine, arginine, or histidine; and amino acid f′ is selected from tyrosine, valine, glycine, arginine, or threonine; and wherein the polypeptide, in association with an antibody light chain, is capable of binding OX40L. In certain embodiments, CDR2a comprises the amino acid sequence f g h i j k l m n o p q r s t g′ wherein f through t is an amino acid sequence as defined above and wherein amino acid g′ is selected from proline, lysine, or serine. In certain embodiments, CDR2a comprises the amino acid sequence f g h i j k l m n o p q r s t g′ h′ wherein f through g′ is an amino acid sequence as defined above and wherein amino acid h′ is selected from valine or glycine. In certain embodiments, CDR2a comprises the amino acid sequence f g h i j k l m n o p q r s t g′ h′ i′ wherein f through h′ is an amino acid sequence as defined above and wherein amino acid i′ is lysine. In certain embodiments, CDR2a comprises the amino acid sequence f g h i j k l m n o p q r s t g′ h′ i′ j′ wherein f through i′ is an amino acid sequence as defined above and wherein amino acid j′ is glycine. In certain embodiments, CDR3a comprises the amino acid sequence u v w x y z a′ b′ c′ d′ e′ f′ k′ wherein u through f′ is an amino acid sequence as defined above and wherein amino acid k′ is selected from aspartic acid, methionine, asparagine, tyrosine, or valine. In certain embodiments, CDR3a comprises the amino acid sequence u v w x y z a′ b′ c′ d′ e′ f′ k′ l′ wherein u through k′ is an amino acid sequence as defined above and wherein amino acid l′ is selected from histidine, aspartic acid, serine, tyrosine, or phenylalanine. In certain embodiments, CDR3a comprises the amino acid sequence u v w x y z a′ b′ c′ d′ e′ f′ k′ l′ m′ wherein u through l′ is an amino acid sequence as defined above and wherein amino acid m′ is selected from valine, aspartic acid, or glycine. In certain embodiments, CDR3a comprises the amino acid sequence u v w x y z a′ b′ c′ d′ e′ f′ k′ l′ m′ n′ wherein u through m′ is an amino acid sequence as defined above and wherein amino acid n′ is selected from phenylalanine, methionine, or tyrosine. In certain embodiments, CDR3a comprises the amino acid sequence u v w x y z a′ b′ c′ d′ e′ f′ k′ l′ m′ n′ o′ wherein u through n′ is an amino acid sequence as defined above and wherein amino acid o′ is aspartic acid. In certain embodiments, CDR3a comprises the amino acid sequence u v w x y z a′ b′ c′ d′ e′ f′ k′ l′ m′ n′ o′ p′ wherein u through o′ is an amino acid sequence as defined above and wherein amino acid p′ is selected from valine or tyrosine.

In certain embodiments, a polypeptide comprises at least two complementarity determining regions (CDR) selected from CDR1a, CDR2a, or CDR3a, wherein the polypeptide, in association with an antibody light chain, is capable of binding OX40L. In certain embodiments, a polypeptide comprises CDR1a, CDR2a, and CDR3a, wherein the polypeptide, in association with an antibody light chain, is capable of binding OX40L.

In certain embodiments, a polypeptide comprises an antibody heavy chain variable region. In certain embodiments, a polypeptide comprises a human antibody heavy chain variable region. In certain embodiments, a polypeptide comprises a heavy chain constant region. In certain embodiments, a polypeptide comprises a human heavy chain constant region. In certain embodiments, a polypeptide comprises an amino acid sequence as set forth in SEQ ID NO. 2; SEQ ID NO. 6; SEQ ID NO. 10; SEQ ID NO. 14; SEQ ID NO. 18; or SEQ ID NO. 22. In certain embodiments, a polypeptide comprises a non-human heavy chain constant region. In certain embodiments, a polypeptide comprises a heavy chain constant region of a species other than human.

In certain embodiments, a polypeptide comprises at least one complementarity determining region (CDR) selected from at least one of amino acids 50 to 54 of SEQ ID NO. 2; amino acids 69 to 87 of SEQ ID NO. 2; amino acids 120 to 135 of SEQ ID NO. 2; amino acids 50 to 54 of SEQ ID NO. 6; amino acids 69 to 84 of SEQ ID NO. 6; amino acids 117 to 134 of SEQ ID NO. 6; amino acids 50 to 54 of SEQ ID NO. 10; amino acids 69 to 85 of SEQ ID NO. 10; amino acids 118 to 135 of SEQ ID NO. 10; amino acids 50 to 54 of SEQ ID NO. 14; amino acids 69 to 84 of SEQ ID NO. 14; amino acids 117 to 131 of SEQ ID NO. 14; amino acids 50 to 54 of SEQ ID NO. 18; amino acids 69 to 87 of SEQ ID NO. 18; amino acids 120 to 133 of SEQ ID NO. 18; amino acids 50 to 54 of SEQ ID NO. 22; amino acids 69 to 87 of SEQ ID NO. 22; or amino acids 120 to 131 of SEQ ID NO. 22, wherein the polypeptide, in association with an antibody light chain, is capable of binding OX40L. In certain embodiments, a polypeptide comprises at least two of the CDRs of SEQ ID NOS. 2, 6, 10, 14, 18, or 22. In certain embodiments, a polypeptide comprises at least three of the CDRs of SEQ ID NOS. 2, 6, 10, 14, 18, or 22.

In certain embodiments, a polypeptide comprises amino acids 50 to 54 of SEQ ID NO. 2, amino acids 69 to 87 of SEQ ID NO. 2, and amino acids 120 to 135 of SEQ ID NO. 2. In certain embodiments, a polypeptide comprises amino acids 50 to 54 of SEQ ID NO. 6, amino acids 69 to 84 of SEQ ID NO. 6, and amino acids 117 to 134 of SEQ ID NO. 6. In certain embodiments, a polypeptide comprises amino acids 50 to 54 of SEQ ID NO. 10, amino acids 69 to 85 of SEQ ID NO. 10, and amino acids 118 to 135 of SEQ ID NO. 10. In certain embodiments, a polypeptide comprises amino acids 50 to 54 of SEQ ID NO. 14, amino acids 69 to 84 of SEQ ID NO. 14, and amino acids 117 to 131 of SEQ ID NO. 14. In certain embodiments, a polypeptide comprises amino acids 50 to 54 of SEQ ID NO. 18, amino acids 69 to 87 of SEQ ID NO. 18, and amino acids 120 to 133 of SEQ ID NO. 18. In certain embodiments, a polypeptide comprises amino acids 50 to 54 of SEQ ID NO. 22, amino acids 69 to 87 of SEQ ID NO. 22, and amino acids 120 to 131 of SEQ ID NO. 22.

In certain embodiments, a polypeptide comprises at least one complementarity determining region (CDR) selected from CDR1b, CDR2b, or CDR3b, wherein CDR1b comprises the amino acid sequence a1 b1 c1 d1 e1 f1 g1 h1 i1 j1 k1, wherein amino acid a1 is arginine; amino acid b1 is selected from alanine or serine; amino acid c1 is serine; amino acid d1 is glutamine; amino acid e1 is selected from glycine or serine; amino acid f1 is selected from isoleucine, valine, or leucine; amino acid g1 is selected from serine or valine; amino acid h1 is selected from asparagine, serine, or histidine; amino acid i1 is selected from histidine, asparagine, serine, or tyrosine; amino acid j1 is selected from leucine, tyrosine, or aspartic acid; and amino acid k1 is selected from valine, leucine, glycine, or asparagine; wherein CDR2b comprises the amino acid sequence l1 m1 n1 o1 p1 q1 r1, wherein amino acid l1 is selected from alanine, glycine, or lysine; amino acid m1 is selected from alanine or lysine; amino acid n1 is selected from serine or phenylalanine; amino acid o1 is selected from threonine, serine, or asparagine; amino acid p1 is selected from leucine or arginine; amino acid q1 is selected from glutamine, alanine, or phenylalanine; and amino acid r1 is selected from serine or threonine; wherein CDR3b comprises the amino acid sequence s1 t1 u1 v1 w1 x1 y1 z1 a1′, wherein amino acid s1 is selected from glutamine or methionine; and amino acid t1 is selected from lysine or glutamine; amino acid u1 is selected from tyrosine, alanine, serine, or phenylalanine; amino acid v1 is selected from asparagine, glycine, threonine, or tyrosine; amino acid w1 is selected from serine, glycine, or glutamine; amino acid x1 is selected from alanine, serine, isoleucine, or threonine; amino acid y1 is selected from proline or leucine; amino acid z1 is selected from leucine, tryptophan, or phenylalanine; and amino acid a1′ is threonine; and wherein the polypeptide, in association with an antibody heavy chain, is capable of binding OX40L. In certain embodiments, CDR1b comprises the amino acid sequence a1 b1 c1 d1 e1 f1 g1 h1 i1 j1 k1 b1′ wherein a1 through k1 is an amino acid sequence as defined above and wherein amino acid b1′ is selected from asparagine or alanine. In certain embodiments, CDR1b comprises the amino acid sequence a1 b1 c1 d1 e1 f1 g1 h1 i1 j1 k1 b1′ c1′ wherein a1 through b1′ is an amino acid sequence as defined above and amino acid c1′ is threonine. In certain embodiments, CDR1b comprises the amino acid sequence a1 b1 c1 d1 e1 f1 g1 h1 i1 j1 k1 b1′ c1′ d1′ wherein a1 through c1′ is an amino acid sequence as defined above and wherein amino acid d1′ is tyrosine. In certain embodiments, CDR1b comprises the amino acid sequence a1 b1 c1 d1 e1 f1 g1 h1 i1 j1 k1 b1′ c1′ d1′ e1′ wherein a1 through d1′ is an amino acid sequence as defined above and wherein amino acid e1′ is leucine. In certain embodiments, CDR1b comprises the amino acid sequence a1 b1 c1 d1 e1 f1 g1 h1 i1 j1 k1 b1′ c1′ d1′ e1′ f1′ wherein a1 through e1′ is an amino acid sequence as defined above and wherein amino acid f1′ is serine.

In certain embodiments, a polypeptide comprises at least two complementarity determining regions (CDR) selected from CDR1b, CDR2b, or CDR3b, wherein the polypeptide, in association with an antibody heavy chain, is capable of binding OX40L. In certain embodiments, a polypeptide comprises CDR1b, CDR2b, and CDR3b, wherein the polypeptide, in association with an antibody heavy chain, is capable of binding OX40L.

In certain embodiments, a polypeptide comprises an antibody light chain variable region. In certain embodiments, a polypeptide comprises a human antibody light chain variable region. In certain embodiments, a polypeptide comprises a light chain constant region. In certain embodiments, a polypeptide comprises a human light chain constant region. In certain embodiments, a polypeptide comprises an amino acid sequence as set forth in SEQ ID NO. 4; SEQ ID NO. 8; SEQ ID NO. 12; SEQ ID NO. 16; or SEQ ID NO. 20. In certain embodiments, a polypeptide comprises a non-human light chain constant region. In certain embodiments, a polypeptide comprises a light chain constant region of a species other than human.

In certain embodiments, a polypeptide which comprises at least one complementarity determining region (CDR) selected from at least one of amino acids 46 to 56 of SEQ ID NO. 4; amino acids 72 to 78 of SEQ ID NO. 4; amino acids 111 to 119 of SEQ ID NO. 4; amino acids 46 to 56 of SEQ ID NO. 8; amino acids 72 to 78 of SEQ ID NO. 8; amino acids 111 to 119 of SEQ ID NO. 8; amino acids 44 to 59 of SEQ ID NO. 12; amino acids 75 to 81 of SEQ ID NO. 12; amino acids 114 to 122 of SEQ ID NO. 12; amino acids 44 to 55 of SEQ ID NO. 16; amino acids 71 to 77 of SEQ ID NO. 16; amino acids 110 to 118 of SEQ ID NO. 16; amino acids 46 to 56 of SEQ ID NO. 20; amino acids 72 to 78 of SEQ ID NO. 20; or amino acids 111 to 119 of SEQ ID NO. 20, wherein the polypeptide, in association with an antibody heavy chain, is capable of binding OX40L. In certain embodiments, a polypeptide comprises at least two of the CDRs of SEQ ID NOS. 4, 8, 12, 16, or 20. In certain embodiments, a polypeptide comprises at least three of the CDRs of SEQ ID NOS. 4, 8, 12, 16, or 20.

In certain embodiments, a polypeptide comprises amino acids 46 to 56 of SEQ ID NO. 4, amino acids 72 to 78 of SEQ ID NO. 4, and amino acids 111 to 119 of SEQ ID NO. 4. In certain embodiments, a polypeptide comprises amino acids 46 to 56 of SEQ ID NO. 8, amino acids 72 to 78 of SEQ ID NO. 8, and amino acids 111 to 119 of SEQ ID NO. 8. In certain embodiments, a polypeptide comprises amino acids 44 to 59 of SEQ ID NO. 12, amino acids 75 to 81 of SEQ ID NO. 12, and amino acids 114 to 122 of SEQ ID NO. 12. In certain embodiments, a polypeptide comprises amino acids 44 to 55 of SEQ ID NO. 16, amino acids 71 to 77 of SEQ ID NO. 16, and amino acids 110 to 118 of SEQ ID NO. 16. In certain embodiments, a polypeptide comprises amino acids 46 to 56 of SEQ ID NO. 20, amino acids 72 to 78 of SEQ ID NO. 20, and amino acids 111 to 119 of SEQ ID NO. 20.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to components that are in a relationship permitting them to function in their intended manner. For example, a control sequence “operably linked” to a coding sequence is ligated in such a way that expression of the coding sequence is achieved under conditions compatible with the control sequences.

The term “control sequence” as used herein refers to polynucleotide sequences which may effect the expression and processing of coding sequences to which they are ligated. The nature of such control sequences may differ depending upon the host organism. According to certain embodiments, control sequences for prokaryotes may include promoter, ribosomal binding site, and transcription termination sequence. According to certain embodiments, control sequences for eukaryotes may include promoters, enhancers, and transcription termination sequence. In certain embodiments, “control sequences” can include leader sequences and/or fusion partner sequences.

Identity and similarity of related polypeptides can be readily calculated by known methods. Such methods include, but are not limited to, those described in Computational Molecular Biology, Lesk, A. M., ed., Oxford University Press, New York (1988); Biocomputing: Informatics and Genome Projects, Smith, D. W., ed., Academic Press, New York (1993); Computer Analysis of Sequence Data, Part 1, Griffin, A. M., and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., M. Stockton Press, New York (1991); and Carillo et al., SIAM J. Applied Math., 48:1073 (1988). In certain embodiments, polypeptides have amino acid sequences that are about 90 percent, or about 95 percent, or about 96 percent, or about 97 percent, or about 98 percent, or about 99 percent identical to amino acid sequences shown in FIGS. 1-11.

Certain methods to determine identity are designed to give the largest match between the sequences tested. Certain, methods to determine identity are described in publicly available computer programs. Certain computer program methods to determine identity between two sequences include, but are not limited to, the GCG program package, including GAP (Devereux et al., Nucl. Acid. Res., 12:387 (1984); Genetics Computer Group, University of Wisconsin, Madison, Wis., BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra (1990)). The well-known Smith Waterman algorithm may also be used to determine identity.

Certain alignment schemes for aligning two amino acid sequences may result in the matching of only a short region of the two sequences, and this small aligned region may have very high sequence identity even though there is no significant relationship between the two full-length sequences. Accordingly, in certain embodiments, the selected alignment method (GAP program) will result in an alignment that spans at least 50 contiguous amino acids of the target polypeptide.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). In certain embodiments, a gap opening penalty (which is calculated as 3× the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see Dayhoff et al., Atlas of Protein Sequence and Structure, 5 (3)(1978) for the PAM 250 comparison matrix; Henikoff et al., Proc. Natl. Acad. Sci USA, 89:10915-10919 (1992) for the BLOSUM 62 comparison matrix) is also used by the algorithm.

In certain embodiments, the parameters for a polypeptide sequence comparison include the following:
Algorithm: Needleman et al., J. Mol. Biol., 48:443-453 (1970);
Comparison matrix: BLOSUM 62 from Henikoff et al., supra (1992);
Gap Penalty: 12
Gap Length Penalty: 4
Threshold of Similarity: 0

The GAP program may be useful with the above parameters. In certain embodiments, the aforementioned parameters are the default parameters for polypeptide comparisons (along with no penalty for end gaps) using the GAP algorithm.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)). Stereoisomers (e.g., D-amino acids) of the twenty conventional amino acids, unnatural amino acids such as α-,α-disubstituted amino acids, N-alkyl amino acids, lactic acid, and other unconventional amino acids may also be suitable components for polypeptides of the present invention. Examples of unconventional amino acids include, but are not limited to: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences.”

Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties.

Naturally occurring residues may be divided into classes based on common side chain properties:
1) hydrophobic: norleucine, Met, Ala, Val, Leu, lie;
2) neutral hydrophilic: Cys, Ser, Thr, Asn, Gln;
3) acidic: Asp, Glu;
4) basic: His, Lys, Arg;
5) residues that influence chain orientation: Gly, Pro; and
6) aromatic: Trp, Tyr, Phe.

For example, non-conservative substitutions may involve the exchange of a member of one of these classes for a member from another class.

In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One may also identify epitopes from primary amino acid sequences on the basis of hydrophilicity. These regions are also referred to as “epitopic core regions.”

Exemplary amino acid substitutions are set forth in Table 1.

TABLE 1
Amino Acid Substitutions

| | | More specific |
|---|---|---|
| Original | Exemplary | exemplary |
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, | Leu |
| | Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan will be able to determine suitable variants of the polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides. In certain embodiments, even areas that may be important for biological activity, including but not limited to the CDRs of an antibody, or that may be important for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues which are important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of an antibody with respect to its three dimensional structure. In certain embodiments, one skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays known to those skilled in the art. For example, the skilled artisan may screen test variants for their ability to bind to OX40L. Such variants could be used to gather information about suitable variants. For example, if one discovered that a change to a particular amino acid residue resulted in destroyed, undesirably reduced, or unsuitable activity, variants with such a change may be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult J., Curr. Op. in Biotech., 7 (4):422-427 (1996), Chou et al., Biochemistry, 13 (2):222-245 (1974); Chou et al., Biochemistry, 113 (2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See Holm et al., Nucl. Acid. Res., 27 (1):244-247 (1999). It has been suggested (Brenner et al., Curr. Op. Struct. Biol., 7 (3):369-376 (1997)) that there are a limited number of folds in a given polypeptide or protein and that once a critical number of structures have been resolved, structural prediction will become dramatically more accurate.

Additional methods of predicting secondary structure include “threading” (Jones, D., Curr. Opin. Struct. Biol., 7 (3):377-87 (1997); Sippl et al., Structure, 4 (1):15-19 (1996)), “profile analysis” (Bowie et al., Science, 253:164-170 (1991); Gribskov et al., Meth. Enzym., 183:146-159 (1990); Gribskov et al., Proc. Nat. Acad. Sci., 84 (13):4355-4358 (1987)), and “evolutionary linkage” (See Holm, supra (1999), and Brenner, supra (1997)).

In certain embodiments, antibody variants include glycosylation variants wherein the number and/or type of glycosylation site has been altered compared to the amino acid sequences of the parent polypeptide. In certain embodiments, protein variants comprise a greater or a lesser number of N-linked glycosylation sites than the native protein. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created.

In certain embodiments, antibody variants include cysteine variants. In certain embodiments, cysteine variants have one or more cysteine residues that are deleted from or that are replaced by another amino acid (e.g., serine) as compared to the parent amino acid sequence. In certain embodiments, cysteine variants have one or more cysteine residues that are added to or that replace another amino acid (e.g., serine) as compared to the parent amino acid sequence. In certain embodiments, cysteine variants may be useful when antibodies are refolded into a biologically active conformation such as after the isolation of insoluble inclusion bodies. In certain embodiments, cysteine variants have fewer cysteine residues than the native protein. In certain embodiments, cysteine variants have more cysteine residues than the native protein. In certain embodiments, cysteine variants have an even number of cysteine residues to minimize interactions resulting from unpaired cysteines.

According to certain embodiments, amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physicochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W.H. Freeman and Company, New York (1984)); Introduction to Protein Structure (C. Branden and J. Tooze, eds., Garland Publishing, New York, N.Y. (1991)); and Thornton et at. Nature 354:105 (1991).

The term “polypeptide fragment” as used herein refers to a polypeptide that has an amino-terminal and/or carboxy-terminal deletion. In certain embodiments, fragments are at least 5 to 467 amino acids long. It will be appreciated that in certain embodiments, fragments are at least 5, 6, 8, 10, 14, 20, 50, 70, 100, 150, 200, 250, 300, 350, 400, or 450 amino acids long.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al. J. Med. Chem. 30:1229 (1987). Such compounds are often developed with the aid of computerized molecular modeling. Peptide mimetics that are structurally similar to therapeutically useful peptides may be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used in certain embodiments to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, by adding internal cysteine residues capable of forming intramolecular disulfide bridges which cyclize the peptide.

The term “isolated antibody” as used herein means an antibody which (1) is free of at least some proteins with which it would normally be found, (2) is essentially free of other proteins from the same source, e.g., from the same species, (3) is expressed by a cell from a different species, or (4) does not occur in nature.

“Antibody” or “antibody peptide(s)” both refer to an intact antibody, or a fragment thereof. In certain embodiments, the antibody fragment may be a binding fragment that competes with the intact antibody for specific binding. The term “antibody” also encompasses polyclonal antibodies and monoclonal antibodies. In certain embodiments, binding fragments are produced by recombinant DNA techniques. In certain embodiments, binding fragments are produced by enzymatic or chemical cleavage of intact antibodies. In certain embodiments, binding fragments are produced by recombinant DNA techniques. Binding fragments include, but are not limited to, Fab, Fab′, F(ab′)2, Fv, Facb, and single-chain antibodies. Non-antigen binding fragments include, but are not limited to, Fc fragments. In certain embodiments, an antibody specifically binds to an epitope that is specifically bound by at least one of Ab A, Ab B, Ab C, Ab D, Ab E, Ab F, Ab G, Ab H, Ab I, or Ab J. The term “antibody” also encompasses anti-idiotypic antibodies that specifically bind to the variable region of another antibody. In certain embodiments, an anti-idiotypic antibody specifically binds to the variable region of an anti-OX40L antibody. In certain embodiments, anti-idiotypic antibodies may be used to detect the presence of a particular anti-OX40L antibody in a sample or to block the activity of an anti-OX40L antibody.

The term “anti-OX40L antibody” as used herein means an antibody that specifically binds to OX40L. In certain embodiments, an anti-OX40L antibody binds to an OX40L epitope to which at least one of Abs A-J binds. In various embodiments, OX40L may be the OX40L of any species, including, but not limited to, human, cynomolgus monkeys, mice, and rabbits. Certain assays for determining the specificity of an antibody are well known to the skilled artisan and include, but are not limited to, ELISA, ELISPOT, western blots, BIAcore assays, solution affinity binding assays, T cell costimulation assays, and T cell migration assays.

In certain embodiments, an anti-OX40L antibody comprises:
(i) a first polypeptide comprising at least one complementarity determining region (CDR) selected from CDR1a, CDR2a, or CDR3a
   wherein CDR1a comprises the amino acid sequence a b c d e, wherein amino acid a is selected from asparagine, threonine, phenylalanine, or serine; amino acid b is selected from alanine or tyrosine; amino acid c is selected from tryptophan, tyrosine, or glycine; amino acid d is selected from methionine or tryptophan; and amino acid e is selected from serine, asparagine, or histidine;
   wherein CDR2a comprises the amino acid sequence f g h i j k l m n o p q r s t, wherein amino acid f is selected from arginine or valine; amino acid g is isoleucine; amino acid h is selected from lysine, tyrosine, or tryptophan; amino acid i is selected from serine, isoleucine, tyrosine, threonine, or arginine; amino acid j is selected from lysine, serine, or aspartic acid; amino acid k is selected from threonine or glycine; amino acid l is selected from aspartic acid, serine, or glutamic acid; amino acid m is selected from glycine, threonine, or asparagine; amino acid n is selected from glycine, asparagine, lysine, or threonine; amino acid o is selected from threonine or tyrosine; amino acid p is selected from threonine, isoleucine, asparagine, or tyrosine; amino acid q is selected from aspartic acid, proline, or alanine; amino acid r is selected from tyrosine, serine, or aspartic acid; amino acid s is selected from glycine, alanine, leucine, or serine; and amino acid t is selected from alanine, lysine, or valine;
   wherein CDR3a comprises the amino acid sequence u v w x y z a′ b′ c′ d′ e′ f′, wherein amino acid u is selected from aspartic acid, glycine, methionine, or serine; amino acid v is selected from arginine, glycine, aspartic acid, tyrosine, or phenylalanine; amino acid w is selected from tyrosine, valine, glycine, or leucine; amino acid x is selected from phenylalanine, aspartic acid, tyrosine, or tryptophan; amino acid y is selected from phenylalanine, aspartic acid, tyrosine, or isoleucine; amino acid z is selected from glycine, tyrosine, proline, valine, or phenylalanine; amino acid a′ is selected from glutamic acid, serine, tyrosine, tryptophan, or alanine; amino acid b′ is selected from phenylalanine, glycine, tyrosine, threonine, or serine; amino acid c′ is selected from proline, tyrosine, serine, lysine, or glycine; amino acid d′ is selected from phenylalanine, tyrosine, or glycine; amino acid e′ is selected from aspartic acid, tyrosine, arginine, or histidine; and amino acid f′ is selected from tyrosine, valine, glycine, arginine, or threonine; and
   wherein the first polypeptide, in association with an antibody light chain, is capable of binding OX40L; and
(ii) a second polypeptide comprising at least one complementarity determining region (CDR) selected from CDR1b, CDR2b, or CDR3b
   wherein CDR1b comprises the amino acid sequence a1 b1 c1 d1 e1 f1 g1 h1 i1 j1 k1, wherein amino acid a1 is arginine; amino acid b1 is selected from alanine or serine; amino acid c1 is serine; amino acid d1 is glutamine; amino acid e1 is selected from glycine or serine; amino acid f1 is selected from isoleucine, valine, or leucine; amino acid g1 is selected from serine or valine; amino acid h1 is selected from asparagine, serine, or histidine; amino acid i1 is selected from histidine, asparagine, serine, or tyrosine; amino acid j1 is selected from leucine, tyrosine, or aspartic acid; and amino acid k1 is selected from valine, leucine, glycine, or asparagine;
   wherein CDR2b comprises the amino acid sequence l1 m1 n1 o1 p1 q1 r1, wherein amino acid l1 is selected from alanine, glycine, or lysine; amino acid m1 is selected from alanine or lysine; amino acid n1 is selected from serine or phenylalanine; amino acid o1 is selected from threonine, serine, or asparagine; amino acid p1 is selected from leucine or arginine; amino acid q1 is selected from glutamine, alanine, or phenylalanine; and amino acid r1 is selected from serine or threonine;
   wherein CDR3b comprises the amino acid sequence s1 t1 u1 v1 w1 x1 y1 z1 a1′, wherein amino acid s1 is selected from glutamine or methionine; and amino acid t1 is selected from lysine or glutamine; amino acid u1 is selected from tyrosine, alanine, serine, or phenylalanine; amino acid v1 is selected from asparagine, glycine, threonine, or tyrosine; amino acid w1 is selected from serine, glycine, or glutamine; amino acid x1 is selected from alanine, serine, isoleucine, or threonine; amino acid y1 is selected from proline or leucine; amino acid z1 is selected from leucine, tryptophan, or phenylalanine; and amino acid a1′ is threonine; and wherein the second polypeptide, in association with an antibody heavy chain, is capable of binding OX40L.

In certain embodiments, an anti-OX40L antibody comprises: a first polypeptide comprising complementarity determining regions (CDRs) as set forth in SEQ ID NO. 2 and a second polypeptide comprising CDRs as set forth in SEQ ID NO. 4; a first polypeptide comprising CDRs as set forth in SEQ ID NO. 6 and a second polypeptide comprising CDRs as set forth in SEQ ID NO. 8; a first polypeptide comprising CDRs as set forth in SEQ ID NO. 10 and a second polypeptide comprising CDRs as set forth in SEQ ID NO. 12; a first polypeptide comprising CDRs as set forth in SEQ ID NO. 14 and a second polypeptide comprising CDRs as set forth in SEQ ID NO. 16; or a first polypeptide comprising CDRs as set forth in SEQ ID NO. 18 and a second polypeptide comprising CDRs as set forth in SEQ ID NO. 20. In certain embodiments, an anti-OX40L antibody comprises a first polypeptide as set forth in paragraph [070] above and a second polypeptide as set forth in paragraph [075] above. In certain embodiments, an anti-OX40L antibody comprises a first polypeptide as set forth in paragraph [071] above and a second polypeptide as set forth in paragraph [076] above. In certain embodiments, an anti-OX40L antibody is a human antibody. In certain embodiments, an anti-OX40L antibody comprises a detectable label. In certain embodiments, an anti-OX40L antibody is a chimeric antibody.

“Chimeric antibody” refers to an antibody that has an antibody variable region of a first species fused to another molecule, for example, an antibody constant region of another second species. In certain embodiments, the first species may be different from the second species. In certain embodiments, the first species may be the same as the second species. In certain embodiments, chimeric antibodies may be made through mutagenesis or CDR grafting to match a portion of the known sequence of anti-OX40L antibody variable regions. CDR grafting typically involves grafting the CDRs from an antibody with desired specificity onto the framework regions (FRs) of another antibody.

A bivalent antibody other than a “multispecific” or “multifunctional” antibody, in certain embodiments, typically is understood to have each of its binding sites identical.

An antibody substantially inhibits adhesion of a ligand to a receptor when an excess of antibody reduces the quantity of receptor bound to the ligand by at least about 20%, 40%, 60%, 80%, 85%, or more (as measured in an in vitro competitive binding assay).

The term “epitope” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. An antibody specifically binds an antigen when it preferentially recognizes its target antigen in a complex mixture of proteins and/or macromolecules. In certain embodiments, an antibody specifically binds an antigen when the dissociation constant is ≦1 μM, in certain embodiments, when the dissociation constant is ≦100 nM, and in certain embodiments, when the dissociation constant is ≦10 nM. In certain embodiments, an antibody specifically binds OX40L.

The term “agent” is used herein to denote a chemical compound, a mixture of chemical compounds, a biological macromolecule, or an extract made from biological materials.

As used herein, the term “label” refers to any molecule that can be detected. In a certain embodiment, an antibody may be labeled by incorporation of a radiolabeled amino acid. In a certain embodiment, biotin moieties that can be detected by marked avidin (e.g., streptavidin containing a fluorescent marker or enzymatic activity that can be detected by optical or calorimetric methods) may be attached to the antibody. In certain embodiments, a label may be incorporated into or attached to another reagent which in turn binds to the antibody of interest. For example, a label may be incorporated into or attached to an antibody that in turn specifically binds the antibody of interest. In certain embodiments, the label or marker can also be therapeutic. Various methods of labeling polypeptides and glycoproteins are known in the art and may be used. Certain general classes of labels include, but are not limited to, enzymatic, fluorescent, chemiluminescent, and radioactive labels. Examples of labels for polypeptides include, but are not limited to, the following: radioisotopes or radionucleoides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., fluorescein isothocyanate (FITC), rhodamine, lanthanide phosphors, phycoerythrin (PE)), enzymatic labels (e.g., horseradish peroxidase, β-galactosidase, luciferase, alkaline phosphatase, glucose oxidase, glucose-6-phosphate dehydrogenase, alcohol dehyrogenase, malate dehyrogenase, penicillinase, luciferase), chemiluminescent, biotinyl groups, predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths to reduce potential steric hindrance.

The term “sample”, as used herein, includes, but is not limited to, any quantity of a substance from a living thing or formerly living thing. Such living things include, but are not limited to, humans, mice, monkeys, rats, rabbits, and other animals. Such substances include, but are not limited to, blood, serum, urine, cells, organs, tissues, bone, bone marrow, lymph nodes, and skin. In certain embodiments, a sample may be from a chemical reaction, including, but not limited to, a protein synthesis reaction.

The term “pharmaceutical agent or drug” as used herein refers to a chemical compound or composition capable of inducing a desired therapeutic effect when properly administered to a patient.

The term “modulator,” as used herein, is a compound that changes or alters the activity or function of a molecule. For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Certain exemplary activities and functions of a molecule include, but are not limited to, binding affinity, enzymatic activity, and signal transduction. Certain exemplary inhibitors include, but are not limited to, proteins, peptides, antibodies, peptibodies, carbohydrates or small organic molecules. Peptibodies are described, e.g., in WO 01/83525.

As used herein, “substantially pure” means an object species is the predominant species present (i.e., on a molar basis it is more abundant than any other individual species in the composition). In certain embodiments, a substantially purified fraction is a composition wherein the object species comprises at least about 50 percent (on a molar basis) of all macromolecular species present. In certain embodiments, a substantially pure composition will comprise more than about 80%, 85%, 90%, 95%, or 99% of all macromolar species present in the composition. In certain embodiments, the object species is purified to essential homogeneity (contaminant species cannot be detected in the composition by conventional detection methods) wherein the composition consists essentially of a single macromolecular species.

The term “patient” includes human and animal subjects.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

According to certain embodiments, a cell line expressing anti-OX40L antibodies is provided.

In certain embodiments, chimeric antibodies that comprise at least a portion of a human sequence and another species' sequence are provided. In certain embodiments, such a chimeric antibody may result in a reduced immune response in a host than an antibody without that host's antibody sequences. For example, in certain instances, an animal of interest may be used as a model for a particular human disease. To study the effect of an antibody on that disease in the animal host, one could use an antibody from a different species. But, in certain instances, such antibodies from another species, may elicit an immune response to the antibodies themselves in the host animal, thus impeding evaluation of these antibodies. In certain embodiments, replacing part of the amino acid sequence of the anti-OX40L antibody with antibody amino acid sequence from the host animal may decrease the magnitude of the host animal's anti-antibody response.

In certain embodiments, a chimeric antibody comprises a heavy chain and a light chain, wherein the variable regions of the light chain and the heavy chain are from a first species and the constant regions of the light chain and the heavy chain are from a second species. In certain embodiments, the antibody heavy chain constant region is an antibody heavy chain constant region of a species other than human. In certain embodiments, the antibody light chain constant region is an antibody light chain constant region of a species other than human. Exemplary antibody constant regions include, but are not limited to, a cynomolgus monkey antibody constant region, a mouse antibody constant region, and a rabbit antibody constant region. Exemplary antibody variable regions include, but are not limited to, a human antibody variable region, a mouse antibody variable region, a pig antibody variable region, a guinea pig antibody variable region, a cynomolgus monkey antibody variable region, and a rabbit antibody variable region. In certain embodiments, the framework regions of the variable region in the heavy chain and light chain may be replaced with framework regions derived from other antibody sequences.

Chimeric antibodies may be produced by methods well known to those of ordinary skill in the art. In certain embodiments, the polynucleotide of the first species encoding the heavy chain variable region and the polynucleotide of the second species encoding the heavy chain constant region can be fused. In certain embodiments, the polynucleotide of the first species encoding the light chain variable region and the nucleotide sequence of the second species encoding the light chain constant region can be fused. In certain embodiments, these fused nucleotide sequences can be introduced into a cell either in a single expression vector (e.g., a plasmid) or in multiple expression vectors. In certain embodiments, a cell comprising at least one expression vector may be used to make polypeptide. In certain embodiments, these fused nucleotide sequences can be introduced into a cell either in separate expression vectors or in a single expression vector. In certain embodiments, the host cell expresses both the heavy chain and the light chain, which combine to produce an antibody. In certain embodiments, a cell comprising at least one expression vector may be used to make an antibody. Exemplary methods for producing and expressing antibodies are discussed below.

In certain embodiments, functional domains, C_{H}1, C_{H}2, C_{H}3, and intervening sequences can be shuffled to create a different antibody constant region. For example, in certain embodiments, such hybrid constant regions can be optimized for half-life in serum, for assembly and folding of the antibody tetramer, and for improved effector function. In certain embodiments, modified antibody constant regions may also be produced by introducing single point mutations into the amino acid sequence of the constant region and testing the resulting antibody for improved qualities, e.g., those listed above.

In certain embodiments, conservative modifications to the heavy and light chains of an anti-OX40L antibody (and corresponding modifications to the encoding nucleotides) will produce antibodies having functional and chemical characteristics similar to those of the original antibody. In contrast, substantial modifications in the functional and/or chemical characteristics of an anti-OX40L antibody may be accomplished by selecting substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a normative residue such that there is little or no effect on the polarity or charge of the amino acid residue at that position. Furthermore, any native residue in the polypeptide may also be substituted with alanine, as has been previously described for “alanine scanning mutagenesis.”

Desired amino acid substitutions (whether conservative or non-conservative) can be determined by those skilled in the art at the time such substitutions are desired. In certain embodiments, amino acid substitutions can be used to identify important residues of the anti-OX40L antibodies, such as those which may increase or decrease the affinity of the antibodies to OX40L or the effector function of the antibodies.

In certain embodiments, the effects of an anti-OX40L antibody may be evaluated by measuring a reduction in the amount of symptoms of the disease. In certain embodiments, the disease of interest may be caused by a pathogen. In certain embodiments, a disease may be established in an animal host by other methods including introduction of a substance (such as a carcinogen) and genetic manipulation. In certain embodiments, effects may be evaluated by detecting one or more adverse events in the animal host. The term “adverse event” includes, but is not limited to, an adverse reaction in an animal host that receives an antibody that is not present in an animal host that does not receive the antibody. In certain embodiments, adverse events include, but are not limited to, a fever, an immune response to an antibody, inflammation, or death of the animal host.

Antibodies specific to an antigen may be produce in a number of ways. In one embodiment, an antigen containing an epitope of interest may be introduced into an animal host (e.g., a mouse), thus producing antibodies specific to that epitope. In certain instances, antibodies specific to an epitope of interest may be obtained from biological samples taken from hosts that were naturally exposed to the epitope. In certain instances, introduction of human immunoglobulin (Ig) loci into mice in which the endogenous Ig genes have been inactivated offers the opportunity to obtain fully human monoclonal antibodies (MAbs).

Naturally Occurring Antibody Structure

Naturally occurring antibody structural units typically comprise a tetramer. Each such tetramer typically is composed of two identical pairs of polypeptide chains, each pair having one full-length “light” chain (in certain embodiments, about 25 kDa) and one full-length “heavy” chain (in certain embodiments, about 50-70 kDa). The term “heavy chain” includes any polypeptide having sufficient variable region sequence to confer specificity for a particular antigen. A full-length heavy chain includes a variable region domain, V_{H}, and three constant region domains, C_{H}1, C_{H}2, and C_{H}3. The V_{H} domain is at the amino-terminus of the polypeptide, and the C_{H}3 domain is at the carboxy-terminus. The term “heavy chain”, as used herein, encompasses a full-length antibody heavy chain and fragments thereof.

The term “light chain” includes any polypeptide having sufficient variable region sequence to confer specificity for a particular epitope. A full-length light chain includes a variable region domain, V_{L}, and a constant region domain, C_{L}. Like the heavy chain, the variable region domain of the light chain is at the amino-terminus of the polypeptide. The term “light chain”, as used herein, encompasses a full-length light chain and fragments thereof.

The amino-terminal portion of each chain typically includes a variable region (V_{H} in the heavy chain and V_{L} in the light chain) of about 100 to 110 or more amino acids that typically is responsible for antigen recognition. The carboxy-terminal portion of each chain typically defines a constant region (C_{H} domains in the heavy chain and C_{L} in the light chain) that may be responsible for effector function. Antibody effector functions include activation of complement and stimulation of opsonophagocytosis. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology Ch. 7 (Paul, W., ed., 2nd ed. Raven Press, N.Y. (1989)). The variable regions of each light/heavy chain pair typically form the antigen binding site.

The variable regions typically exhibit the same general structure of relatively conserved framework regions (FR) joined by three hypervariable regions, also called complementarity determining regions or CDRs. The CDRs from the heavy and light chains of each pair typically are aligned by the framework regions, which may enable binding to a specific epitope. From N-terminal to C-terminal, both light and heavy chain variable regions typically comprise the domains FR1, CDR1, FR2, CDR2, FR3, CDR3, and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

As discussed above, there are several types of antibody fragments. A Fab fragment is comprised of one light chain and the C_{H}1 and variable regions of one heavy chain. The heavy chain of a Fab molecule cannot form a disulfide bond with another heavy chain molecule. A Fab′ fragment contains one light chain and one heavy chain that contains more of the constant region, between the C_{H}1 and C_{H}2 domains, such that an interchain disulfide bond can be formed between two heavy chains to form a F(ab′)2 molecule. A Facb fragment is similar to a F(ab′)2 molecule, except the constant region in the heavy chains of the molecule extends to the end of the C_{H}2 domain. The Fv region comprises the variable regions from both the heavy and light chains, but lacks the constant regions. Single-chain antibodies are Fv molecules in which the heavy and light chain variable regions have been connected by a flexible linker to form a single polypeptide chain which forms an antigen-binding region. Single chain antibodies are discussed in detail, e.g., in WO 88/01649 and U.S. Pat. Nos. 4,946,778 and 5,260,203. A Fc fragment contains the C_{H}2 and C_{H}3 domains of the heavy chain and contains more of the constant region, between the C_{H}1 and C_{H}2 domains, such that an interchain disulfide bond can be formed between two heavy chains.

Bispecific or Bifunctional Antibodies

A bispecific or bifunctional antibody typically is an artificial hybrid antibody having two different heavy/light chain pairs and two different binding sites. Bispecific antibodies may be produced by a variety of methods including, but not limited to, fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992).

Certain Preparation of Antibodies

In certain embodiments, antibodies can be expressed in cell lines other than hybridoma cell lines. In certain embodiments, sequences encoding particular antibodies, including chimeric antibodies, can be used for transformation of a suitable mammalian host cell. According to certain embodiments, transformation can be by any known method for introducing polynucleotides into a host cell, including, for example packaging the polynucleotide in a virus (or into a viral vector) and transducing a host cell with the virus or by transfecting a vector using procedures known in the art, as exemplified by U.S. Pat. Nos. 4,399,216; 4,912,040; 4,740,461; and 4,959,455.

In certain embodiments, an expression vector comprises any of the polynucleotide sequences discussed herein. In certain embodiments, a method of making a polypeptide comprising producing the polypeptide in a cell comprising any of the above expression vectors in conditions suitable to express the polynucleotide contained therein to produce the polypeptide is provided.

In certain embodiments, an expression vector comprises a polynucleotide comprising a sequence encoding a polypeptide comprising at least one complementarity determining region (CDR) selected from CDR1a, CDR2a, or CDR3a, wherein CDR1a comprises the amino acid sequence a b c d e, wherein amino acid a is selected from asparagine, threonine, phenylalanine, or serine; amino acid b is selected from alanine or tyrosine; amino acid c is selected from tryptophan, tyrosine, or glycine; amino acid d is selected from methionine or tryptophan; and amino acid e is selected from serine, asparagine, or histidine; wherein CDR2a comprises the amino acid sequence f g h i j k l m n o p q r s t, wherein amino acid f is selected from arginine or valine; amino acid g is isoleucine; amino acid h is selected from lysine, tyrosine, or tryptophan; amino acid i is selected from serine, isoleucine, tyrosine, threonine, or arginine; amino acid j is selected from lysine, serine, or aspartic acid; amino acid k is selected from threonine or glycine; amino acid l is selected from aspartic acid, serine, or glutamic acid; amino acid m is selected from glycine, threonine, or asparagine; amino acid n is selected from glycine, asparagine, lysine, or threonine; amino acid o is selected from threonine or tyrosine; amino acid p is selected from threonine, isoleucine, asparagine, or tyrosine; amino acid q is selected from aspartic acid, proline, or alanine; amino acid r is selected from tyrosine, serine, or aspartic acid; amino acid s is selected from glycine, alanine, leucine, or serine; and amino acid t is selected from alanine, lysine, or valine; wherein CDR3a comprises the amino acid sequence u v w x y z a′ b′ c′ d′ e′ f′, wherein amino acid u is selected from aspartic acid, glycine, methionine, or serine; amino acid v is selected from arginine, glycine, aspartic acid, tyrosine, or phenylalanine; amino acid w is selected from tyrosine, valine, glycine, or leucine; amino acid x is selected from phenylalanine, aspartic acid, tyrosine, or tryptophan; amino acid y is selected from phenylalanine, aspartic acid, tyrosine, or isoleucine; amino acid z is selected from glycine, tyrosine, proline, valine, or phenylalanine; amino acid a′ is selected from glutamic acid, serine, tyrosine, tryptophan, or alanine; amino acid b′ is selected from phenylalanine, glycine, tyrosine, threonine, or serine; amino acid c′ is selected from proline, tyrosine, serine, lysine, or glycine; amino acid d′ is selected from phenylalanine, tyrosine, or glycine; amino acid e′ is selected from aspartic acid, tyrosine, arginine, or histidine; and amino acid f′ is selected from tyrosine, valine, glycine, arginine, or threonine; and wherein the polypeptide, in association with an antibody light chain, is capable of binding OX40L. In certain embodiments, an expression vector comprises a polynucleotide comprising a sequence encoding CDR2a comprising the amino acid sequence f g h i j k l m n o p q r s t g′ wherein f through t is an amino acid sequence as defined above and wherein amino acid g′ is selected from proline, lysine, or serine. In certain embodiments, an expression vector comprises a polynucleotide comprising a sequence encoding CDR2a comprising the amino acid sequence f g h i j k l m n o p q r s t g′ h′ wherein f through g′ is an amino acid sequence as defined above and amino acid h′ is selected from valine or glycine. In certain embodiments, an expression vector comprises a polynucleotide comprising a sequence encoding CDR2a comprising the amino acid sequence f g h i j k l m n o p q r s t g′ h′ i′ wherein f through h′ is an amino acid sequence as defined above and wherein amino acid i′ is lysine. In certain embodiments, an expression vector comprises a polynucleotide comprising a sequence encoding CDR2a comprising the amino acid sequence f g h i j k l m n o p q r s t g′ h′ i′ j′ wherein f through i′ is an amino acid sequence as defined above and wherein amino acid j′ is glycine. In certain embodiments, an expression vector comprises a polynucleotide comprising a sequence encoding CDR3a comprising the amino acid sequence u v w x y z a′ b′ c′ d′ e′ f′ k′ wherein u through f′ is an amino acid sequence as defined above and wherein amino acid k′ is selected from aspartic acid, methionine, asparagine, tyrosine, or valine. In certain embodiments, an expression vector comprises a polynucleotide comprising a sequence encoding CDR3a comprising the amino acid sequence u v w x y z a′ b′ c′ d′ e′ f′ k′ l′ wherein u through k′ is an amino acid sequence as defined above and wherein amino acid l′ is selected from histidine, aspartic acid, serine, tyrosine, or phenylalanine. In certain embodiments, an expression vector comprises a polynucleotide comprising a sequence encoding CDR3a comprising the amino acid sequence u v w x y z a′ b′ c′ d′ e′ f′ k′ l′ m′ wherein u through l′ is an amino acid sequence as defined above and wherein amino acid m′ is selected from valine, aspartic acid, or glycine. In certain embodiments, an expression vector comprises a polynucleotide comprising a sequence encoding CDR3a comprising the amino acid sequence u v w x y z a′ b′ c′ d′ e′ f′ k′ l′ m′ n′ wherein u through m′ is an amino acid sequence as defined above and wherein amino acid n′ is selected from phenylalanine, methionine, or tyrosine. In certain embodiments, an expression vector comprises a polynucleotide comprising a sequence encoding CDR3a comprising the amino acid sequence u v w x y z a′ b′ c′ d′ e′ f′ k′ l′ m′ n′ o′ wherein u through n′ is an amino acid sequence as defined above and wherein amino acid o′ is aspartic acid. In certain embodiments, an expression vector comprises a polynucleotide comprising a sequence encoding CDR3a comprising the amino acid sequence u v w x y z a′ b′ c′ d′ e′ f′ k′ l′ m′ n′ o′ p′ wherein u through o′ is an amino acid sequence as defined above and wherein amino acid p′ is selected from valine or tyrosine. In certain embodiments, a method of making a polypeptide comprising producing the polypeptide in a cell comprising the above expression vector in conditions suitable to express the polynucleotide contained therein to produce the polypeptide is provided.

In certain embodiments, an expression vector comprises a polynucleotide comprising a sequence encoding a polypeptide comprising at least one complementarity determining region (CDR) selected from CDR1b, CDR2b, or CDR3b, wherein CDR1b comprises the amino acid sequence a1 b1 c1 d1 e1 f1 g1 h1 i1 j1 k1, wherein amino acid a1 is arginine; amino acid b1 is selected from alanine or serine; amino acid c1 is serine; amino acid d1 is glutamine; amino acid e1 is selected from glycine or serine; amino acid f1 is selected from isoleucine, valine, or leucine; amino acid g1 is selected from serine or valine; amino acid h1 is selected from asparagine, serine, or histidine; amino acid i1 is selected from histidine, asparagine, serine, or tyrosine; amino acid j1 is selected from leucine, tyrosine, or aspartic acid; and amino acid k1 is selected from valine, leucine, glycine, or asparagine; wherein CDR2b comprises the amino acid sequence l1 m1 n1 o1 p1 q1 r1, wherein amino acid l1 is selected from alanine, glycine, or lysine; amino acid m1 is selected from alanine or lysine; amino acid n1 is selected from serine or phenylalanine; amino acid o1 is selected from threonine, serine, or asparagine; amino acid p1 is selected from leucine or arginine; amino acid q1 is selected from glutamine, alanine, or phenylalanine; and amino acid r1 is selected from serine or threonine; wherein CDR3b comprises the amino acid sequence s1 t1 u1 v1 w1 x1 y1 z1 a1′, wherein amino acid s1 is selected from glutamine or methionine; and amino acid t1 is selected from lysine or glutamine; amino acid u1 is selected from tyrosine, alanine, serine, or phenylalanine; amino acid v1 is selected from asparagine, glycine, threonine, or tyrosine; amino acid w1 is selected from serine, glycine, or glutamine; amino acid x1 is selected from alanine, serine, isoleucine, or threonine; amino acid y1 is selected from proline or leucine; amino acid z1 is selected from leucine, tryptophan, or phenylalanine; and amino acid a1′ is threonine; and wherein the polypeptide, in association with an antibody heavy chain, is capable of binding OX40L. In certain embodiments, an expression vector comprises a polynucleotide comprising a sequence encoding CDR1b comprising the amino acid sequence a1 b1 c1 d1 e1 f1 g1 h1 i1 j1 k1 b1′ wherein a1 through k1 is an amino acid sequence as defined above and wherein amino acid b1′ is selected from asparagine or alanine. In certain embodiments, an expression vector comprises a polynucleotide comprising a sequence encoding CDR1b comprising the amino acid sequence a1 b1 c1 d1 e1 f1 g1 h1 i1 j1 k1 b1′ c1′ wherein a1 through b1′ is an amino acid sequence as defined above and wherein amino acid c1′ is threonine. In certain embodiments, an expression vector comprises a polynucleotide comprising a sequence encoding CDR1b comprising the amino acid sequence a1 b1 c1 d1 e1 f1 g1 h1 i1 j1 k1 b1′ c140 d1′ wherein a1 through c1′ is an amino acid sequence as defined above and amino acid d1′ is tyrosine. In certain embodiments, an expression vector comprises a polynucleotide comprising a sequence encoding CDR1b comprising the amino acid sequence a1 b1 c1 d1 e1 f1 g1 h1 i1 j1 k1 b1′ c1′ d1′ e1′ wherein a1 through d1′ is an amino acid sequence as defined above and wherein amino acid e1′ is leucine. In certain embodiments, an expression vector comprises a polynucleotide comprising a sequence encoding CDR1b comprising the amino acid sequence a1 b1 c1 d1 e1 f1 g1 h1 i1 j1 k1 b1′ c1′ d1′ e1′ f1′ wherein a1 through e1′ is an amino acid sequence as defined above and wherein amino acid f1′ is serine. In certain embodiments, a method of making a polypeptide comprising producing the polypeptide in a cell comprising the above expression vector in conditions suitable to express the polynucleotide contained therein to produce the polypeptide is provided. In certain embodiments, a cell comprising at least one of the above expression vectors is provided. In certain embodiments, a method of making an polypeptide comprising producing the polypeptide in a cell comprising the above expression vector in conditions suitable to express the polynucleotide contained therein to produce the polypeptide is provided.

In certain embodiments, an expression vector expresses an anti-OX40L antibody heavy chain. In certain embodiments, an expression vector expresses an anti-OX40L antibody light chain. In certain embodiments, an expression vector expresses both an anti-OX40L antibody heavy chain and an anti-OX40L antibody light chain. In certain embodiments, a method of making an anti-OX40L antibody comprising producing the antibody in a cell comprising at least one of the expression vectors described herein in conditions suitable to express the polynucleotides contained therein to produce the antibody is provided.

In certain embodiments, the transfection procedure used may depend upon the host to be transformed. Certain methods for introduction of heterologous polynucleotides into mammalian cells are known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

Certain mammalian cell lines available as hosts for expression are known in the art and include, but are not limited to, many immortalized cell lines available from the American Type Culture Collection (ATCC), including but not limited to Chinese hamster ovary (CHO) cells, E5 cells, HeLa cells, baby hamster kidney (BHK) cells, monkey kidney cells (COS), human hepatocellular carcinoma cells (e.g., Hep G2), NS0 cells, SP20 cells, Per C6 cells, 293 cells, and a number of other cell lines. In certain embodiments, cell lines may be selected through determining which cell lines have high expression levels and produce antibodies with constitutive antigen binding properties.

In certain embodiments, the vectors that may be transfected into a host cell comprise control sequences that are operably linked to a polynucleotide encoding an anti-OX40L antibody. In certain embodiments, control sequences facilitate expression of the linked polynucleotide, thus resulting in the production of the polypeptide encoded by the linked polynucleotide. In certain embodiments, the vector also comprises polynucleotide sequences that allow chromosome-independent replication in the host cell. Exemplary vectors include, but are not limited to, plasmids (e.g., BlueScript, puc, etc.), cosmids, and YACS.

Certain Antibody Uses

According to certain embodiments, antibodies are useful for detecting a particular antigen in a sample. In certain embodiments, this allows the identification of cells or tissues which produce the protein. For example, in certain embodiments, anti-OX40L antibodies may be used to detect the presence of OX40L in a sample. In certain embodiments, a method for detecting the presence or absence of OX40L in a sample comprises (a) combining an anti-OX40L antibody and the sample; (b) separating antibodies bound to an antigen from unbound antibodies; and (c) detecting the presence or absence of antibodies bound to the antigen.

Assays in which an antibody may be used to detect the presence or absence of an antigen include, but are not limited to, an ELISA and a western blot. In certain embodiments, an anti-OX40L antibody may be labeled. In certain embodiments, an anti-OX40L antibody may be detected by a labeled antibody that binds to the anti-OX40L antibody. In certain embodiments, a kit for detecting the presence or absence of OX40L in a sample is provided. In certain embodiments, the kit comprises an anti-OX40L antibody and reagents for detecting the antibody. In certain embodiments, the kit comprises an anti-OX40L antibody, as described in paragraph [0109] above and reagents for detecting the antibody. In certain embodiments, the kit comprises an anti-OX40L antibody as described in paragraph [0110] above and reagents for detecting the antibody.

In certain embodiments, antibodies may be used to substantially isolate a chemical moiety such as, but not limited to, a protein. In certain embodiments, the antibody is attached to a “substrate,” which is a supporting material used for immobilizing the antibody. Substrates include, but are not limited to, tubes, plates (i.e., multi-well plates), beads such as microbeads, filters, balls, and membranes. In certain embodiments, a substrate can be made of water-insoluble materials such as, but not limited to, polycarbonate resin, silicone resin, or nylon resin. Exemplary substrates for use in affinity chromatography include, but are not limited to, cellulose, agarose, polyacrylamide, dextran, polystyrene, polyvinyl alcohol, and porous silica. There are many commercially available chromatography substrates that include, but are not limited to, Sepharose 2b, Sepharose 4B, Sepharose 6B and other forms of Sepharose (Pharmacia); Bio-Gel (and various forms of Bio-Gel such as Biogel A, P, or CM), Cellex (and various forms of Cellex such as Cellex AE or Cellex-CM), Chromagel A, Chromagel P and Enzafix (Wako Chemical Indus.). The use of antibody affinity columns is known to a person of ordinary skill in the art. In certain embodiments, a method for isolating OX40L comprises (a) attaching an OX40L antibody to a substrate; (b) exposing a sample containing OX40L to the antibody of part (a); and (c) isolating OX40L. In certain embodiments, a method for isolating OX40L comprises (a) attaching an OX40L antibody as described in paragraph [0109] above to a substrate; (b) exposing a sample containing OX40L to the antibody of part (a); and (c) isolating OX40L. In certain embodiments, a method for isolating OX40L comprises (a) attaching an OX40L antibody as described in paragraph [0110] above to a substrate; (b) exposing a sample containing OX40L to the antibody of part (a); and (c) isolating OX40L. In certain embodiments, a kit for isolating OX40L is provided. In certain embodiments, the kit comprises an anti-OX40L antibody attached to a substrate and reagents for isolating OX40L. In certain embodiments, the kit comprises an anti-OX40L antibody as described in paragraph [0109] above attached to a substrate and reagents for isolating OX40L. In certain embodiments, the kit comprises an anti-OX40L antibody as described in paragraph [0110] above attached to a substrate and reagents for isolating OX40L.

The term “affinity chromatography” as used herein means a method of separating or purifying the materials of interest in a sample by utilizing the interaction (e.g., the affinity) between a pair of materials, such as an antigen and an antibody, an enzyme and a substrate, or a receptor and a ligand.

In certain embodiments, antibodies which bind to a particular protein and block interaction with other binding compounds may have therapeutic use. In this application, when discussing the use of anti-OX40L antibodies to treat diseases or conditions, such use may include use of the anti-OX40L antibodies themselves; compositions comprising anti-OX40L antibodies; and/or combination therapies comprising anti-OX40L antibodies and one or more additional active ingredients. When anti-OX40L antibodies are used to “treat” a disease or condition, such treatment may or may not include prevention of the disease or condition. For example, anti-OX40L antibodies, as shown in the examples below, can block the interaction of OX40L with its receptor, OX40R. Because OX40L is associated with inflammatory immune responses, in certain embodiments, anti-OX40L antibodies may have therapeutic use in treating a variety of diseases including, but not limited to, those diseases associated with inflammation. These diseases include, but are not limited to, rheumatoid arthritis, osteoarthritis, graft-versus-host disease, inflammatory bowel disease, Crohn's Disease, ulcerative colitis, multiple sclerosis, psoriasis, and proliferative lupus nephritis.

In certain embodiments, anti-OX40L antibodies may be used to treat bacterial, viral or protozoal infections, and complications resulting therefrom. Bacterial diseases include, but are not limited to, Mycoplasma pneumonia. In certain embodiments, anti-OX40L antibodies may be used, e.g., in combination with ENBREL™, to treat HIV infection and its associated disease, AIDS, and conditions associated with AIDS and/or related to AIDS, such as AIDS dementia complex, AIDS associated wasting, lipidistrophy due to antiretroviral therapy; CMV (cytomegalovirus) and Kaposi's sarcoma. In certain embodiments, anti-OX40L antibodies may be used to treat protozoal diseases, including, but not limited to, malaria and schistosomiasis. In certain embodiments, anti-OX40L antibodies may be used to treat erythema nodosum leprosum; bacterial or viral meningitis; tuberculosis, including pulmonary tuberculosis; and pneumonitis secondary to a bacterial or viral infection. In certain embodiments, anti-OX40L antibodies may be used to treat louse-borne relapsing fevers, such as that caused by Borrelia recurrentis. In certain embodiments, anti-OX40L antibodies may be used to treat conditions caused by Herpes viruses, such as herpetic stromal keratitis, corneal lesions; and virus-induced corneal disorders. In certain embodiments, anti-OX40L antibodies may be used to treat human papillomavirus infections. In certain embodiments, anti-OX40L antibodies may be used to treat influenza infection and infectious mononucleosis.

In certain embodiments, anti-OX40L antibodies may be used to treat chronic pain conditions, including, but not limited to, chronic pelvic pain, including chronic prostatitis/pelvic pain syndrome. In certain embodiments, anti-OX40L antibodies may be used to treat post-herpetic pain.

In certain embodiments, anti-OX40L antibodies may be used to treat various disorders of the endocrine system. In certain embodiments, anti-OX40L antibodies may be used to treat juvenile onset diabetes (includes autoimmune diabetes mellitus and insulin-dependent types of diabetes) and/or maturity onset diabetes (includes non-insulin dependent and obesity-mediated diabetes). In certain embodiments, anti-OX40L antibodies may be used with TNF inhibitors such as ENBREL™ or other active agents described herein to treat juvenile onset diabetes (includes autoimmune diabetes mellitus and insulin-dependent types of diabetes) and/or maturity onset diabetes (includes non-insulin dependent and obesity-mediated diabetes). In certain embodiments, anti-OX40L antibodies may be used to treat secondary conditions associated with diabetes, such as diabetic retinopathy, kidney transplant rejection in diabetic patients, obesity-mediated insulin resistance, and renal failure, which itself may be associated with proteinurea and hypertension. In certain embodiments, anti-OX40L antibodies may be used to treat other endocrine disorders, including, but not limited to, polycystic ovarian disease, X-linked adrenoleukodystrophy, hypothyroidism and thyroiditis, including Hashimoto's thyroiditis (i.e., autoimmune thyroiditis). In certain embodiments, anti-OX40L antibodies may be used to treat medical conditions associated with thyroid cell dysfunction, including, but not limited to, euthyroid sick syndrome.

In certain embodiments, anti-OX40L antibodies may be used to treat conditions of the gastrointestinal system including, but not limited to, coeliac disease. In certain embodiments, anti-OX40L antibodies may be used with TNF inhibitors such as ENBREL™ or other active agents described herein are suitable to treat coeliac disease. In certain embodiments, anti-OX40L antibodies may be used to treat gastrointestinal diseases including, but not limited to, Crohn's disease; ulcerative colitis; idiopathic gastroparesis; pancreatitis, including chronic pancreatitis; acute pancreatitis, inflammatory bowel disease and ulcers, including gastric and duodenal ulcers.

In certain embodiments, anti-OX40L antibodies may be used to treat disorders of the genitourinary system. In certain embodiments, anti-OX40L antibodies may be used to treat glomerulonephritis, including autoimmune glomerulonephritis, glomerulonephritis due to exposure to toxins or glomerulonephritis secondary to infections with haemolytic streptococci or other infectious agents. In certain embodiments, anti-OX40L antibodies may be used to treat genitourinary diseases including, but not limited to, uremic syndrome and its clinical complications (for example, renal failure, anemia, and hypertrophic cardiomyopathy), including uremic syndrome associated with exposure to environmental toxins, drugs or other causes. In certain embodiments, anti-OX40L antibodies may be used to treat complications that arise from inflammation of the gallbladder wall that leads to alteration in absorptive function. Such complications include, but are not limited to, cholelithiasis (gallstones) and choliedocholithiasis (bile duct stones) and the recurrence of cholelithiasis and choliedocholithiasis. In certain embodiments, anti-OX40L antibodies may be used to treat complications of hemodialysis; prostate conditions, including benign prostatic hypertrophy, nonbacterial prostatitis and chronic prostatitis; and complications of hemodialysis.

In certain embodiments, anti-OX40L antibodies may be used to treat various hematologic and oncologic disorders. In certain embodiments, anti-OX40L antibodies may be used to treat various forms of cancer, including, but not limited to, acute myelogenous leukemia, chronic myelogenous leukemia, Epstein-Barr virus-positive nasopharyngeal carcinoma, glioma, colon, stomach, prostate, renal cell, cervical and ovarian cancers, lung cancer (SCLC and NSCLC), including, but not limited to, cancer-associated cachexia, fatigue, asthenia, paraneoplastic syndrome of cachexia and hypercalcemia. In certain embodiments, anti-OX40L antibodies may be used to treat solid tumors, including sarcoma, osteosarcoma, and carcinoma, such as adenocarcinoma (for example, breast cancer) and squamous cell carcinoma. In certain embodiments, anti-OX40L antibodies may be used to treat esophageal cancer, gastric cancer, gall bladder carcinoma, leukemia, including acute myelogenous leukemia, chronic myelogenous leukemia, myeloid leukemia, chronic or acute lymphoblastic leukemia and hairy cell leukemia. In certain embodiments, anti-OX40L antibodies may be used to treat other malignancies with invasive metastatic potential, including, but not limited to, multiple myeloma. In certain embodiments, anti-OX40L antibodies may be used to treat anemias and hematologic disorders, including, but not limited to, chronic idiopathic neutropenia, anemia of chronic disease, aplastic anemia, including Fanconi's aplastic anemia; idiopathic thrombocytopenic purpura (ITP); thrombotic thrombocytopenic purpura, myelodysplastic syndromes (including refractory anemia, refractory anemia with ringed sideroblasts, refractory anemia with excess blasts, refractory anemia with excess blasts in transformation); myelofibrosis/myeloid metaplasia; and sickle cell vasocclusive crisis.

In certain embodiments, anti-OX40L antibodies may be used to treat various lymphoproliferative disorders. In certain embodiments, anti-OX40L antibodies may be used to treat autoimmune lymphoproliferative syndrome (ALPS), chronic lymphoblastic leukemia, hairy cell leukemia, chronic lymphatic leukemia, peripheral T cell lymphoma, small lymphocytic lymphoma, mantle cell lymphoma, follicular lymphoma, Burkitt's lymphoma, Epstein-Barr virus-positive T cell lymphoma, histiocytic lymphoma, Hodgkin's disease, diffuse aggressive lymphoma, acute lymphatic leukemias, T gamma lymphoproliferative disease, cutaneous B cell lymphoma, cutaneous T cell lymphoma (i.e., mycosis fungoides) and Sezary syndrome.

In certain embodiments, anti-OX40L antibodies may be used to treat hereditary conditions. In certain embodiments, anti-OX40L antibodies may be used to treat diseases including, but not limited to, Gaucher's disease, Huntington's disease, linear IgA disease, and muscular dystrophy.

In certain embodiments, anti-OX40L antibodies may be used to treat injuries to the head or spinal cord including, but not limited to, subdural hematoma due to trauma to the head. In certain embodiments, anti-OX40L antibodies may be used to treat head injuries and spinal chord injuries. In certain embodiments, anti-OX40L antibodies may be used to treat cranial neurologic damage and/or cervicogenic headache. In certain embodiments, anti-OX40L antibodies may be used to treat neurological side effects associated with brain irradiation.

In certain embodiments, anti-OX40L antibodies may be used to treat conditions of the liver. In certain embodiments, anti-OX40L antibodies may be used to treat hepatitis, including acute alcoholic hepatitis, acute drug-induced or viral hepatitis, hepatitis A, B and C, sclerosing cholangitis and inflammation of the liver due to unknown causes. In certain embodiments, anti-OX40L antibodies may be used to treat hepatic sinusoid epithelium. In certain embodiments, anti-OX40L antibodies may be used to treat various disorders that involve hearing loss including, but not limited to, cochlear nerve-associated hearing loss that is thought to result from an autoimmune process, i.e., autoimmune hearing loss. This condition currently is treated with steroids, methotrexate and/or cyclophosphamide. In certain embodiments, anti-OX40L antibodies may be used to treat Meniere's syndrome and cholesteatoma, a middle ear disorder often associated with hearing loss.

In certain embodiments, anti-OX40L antibodies may be used to treat non-arthritic medical conditions of the bones and joints, including, but not limited to, osteoclast disorders that lead to bone loss, such as but not limited to osteoporosis, including post-menopausal osteoporosis, osteoarthritis, periodontitis resulting in tooth loosening or loss, and prosthesis loosening after joint replacement (generally associated with an inflammatory response to wear debris). This latter condition also is called “orthopedic implant osteolysis.” In certain embodiments, anti-OX40L antibodies may be used to treat temporal mandibular joint dysfunction (TMJ).

In certain embodiments, anti-OX40L antibodies may be used to treat pulmonary diseases including, but not limited to, adult respiratory distress syndrome (ARDS), acute respiratory distress syndrome and acute lung injury caused by a variety of conditions, including exposure to toxic chemicals, pancreatitis, trauma or other causes of inflammation. In certain embodiments, anti-OX40L antibodies may be used to treat broncho-pulmonary dysplasia (BPD); chronic obstructive pulmonary diseases (e.g. emphysema and chronic bronchitis), and chronic fibrotic lung disease of preterm infants. In certain embodiments, anti-OX40L antibodies may be used to treat occupational lung diseases, including asbestosis, coal worker's pneurnoconiosis, silicosis or similar conditions associated with long-term exposure to fine particles. In certain embodiments, anti-OX40L antibodies may be used to treat bronchioliterans organizing pneumonia, pulmonary fibrosis, including, but not limited to, idiopathic pulmonary fibrosis and radiation-induced pulmonary fibrosis; pulmonary sarcoidosis; and allergies, including allergic rhinitis, contact dermatitis, atopic dermatitis and asthma.

In certain embodiments, anti-OX40L antibodies may be used to treat a variety of rheumatic disorders including, but not limited to, adult and juvenile rheumatoid arthritis; scleroderma; systemic lupus erythematosus; gout; osteoarthritis; polymyalgia rheumatica; seronegative spondylarthropathies, including ankylosing spondylitis, and Reiter's disease.

In certain embodiments, anti-OX40L antibodies may be used to treat psoriatic arthritis and chronic Lyme arthritis. In certain embodiments, anti-OX40L antibodies may be used to treat Still's disease and uveitis associated with rheumatoid arthritis. In certain embodiments, anti-OX40L antibodies may be used to treat disorders resulting in inflammation of the voluntary muscle and other muscles, including dermatomyositis, inclusion body myositis, polymyositis, and lymphangioleimyomatosis.

In certain embodiments, anti-OX40L antibodies may be used to treat primary amyloidosis. In certain embodiments, anti-OX40L antibodies may be used to treat secondary amyloidosis that is characteristic of various conditions. Such conditions include, but are not limited to, Alzheimer's disease, secondary reactive amyloidosis; Down's syndrome; and dialysis-associated amyloidosis. In certain embodiments, anti-OX40L antibodies may be used to treat inherited periodic fever syndromes, including familial Mediterranean fever, hyperimmunoglobulin D and periodic fever syndrome and TNF-receptor associated periodic syndromes (TRAPS).

In certain embodiments, anti-OX40L antibodies may be used to treat disorders involving the skin or mucous membranes. Such disorders include, but are not limited to, acantholytic diseases, including Darier's disease, keratosis follicularis and pemphigus vulgaris. In certain embodiments, anti-OX40L antibodies may be used to treat acne; acne rosacea; alopecia greata; aphthous stomatitis; bullous pemphigoid; burns; eczema; erythema, including erythema multiforme and erythema multiforme bullosum (Stevens-Johnson syndrome); inflammatory skin disease; lichen planus; linear IgA bullous disease (chronic bullous dermatosis of childhood); loss of skin elasticity; mucosal surface ulcers, including gastric ulcers; neutrophilic dermatitis (Sweet's syndrome); dermatomyositis, pityriasis rubra pilaris; psoriasis; pyoderma gangrenosum; multicentric reticulohistiocytosis; and toxic epidermal necrolysis. In certain embodiments, anti-OX40L antibodies may be used to treat dermatitis herpetiformis. In certain embodiments, anti-OX40L antibodies may be used to treat disorders associated with transplantation. Such disorders include, but are not limited to, graft-versus-host disease, and complications resulting from solid organ transplantation, such as heart, liver, skin, kidney, lung (lung transplant airway obliteration) or other transplants, including bone marrow transplants.

In certain embodiments, anti-OX40L antibodies may be used to treat ocular disorders, including, but not limited to, rhegmatogenous retinal detachment, and inflammatory eye disease, including inflammatory eye disease associated with smoking and macular degeneration.

In certain embodiments, anti-OX40L antibodies may be used to treat disorders that affect the female reproductive system. Examples include, but are not limited to, multiple implant failure/infertility; fetal loss syndrome or IV embryo loss (spontaneous abortion); preeclamptic pregnancies or eclampsia; endometriosis, chronic cervicitis, and pre-term labor.

In certain embodiments, anti-OX40L antibodies may be used to treat obesity, including to bring about a decrease in leptin formation. In certain embodiments, anti-OX40L antibodies may be used to treat sciatica, symptoms of aging, severe drug reactions (for example, 11-2 toxicity or bleomycin-induced pneumopathy and fibrosis), or to suppress the inflammatory response prior, during or after the transfusion of allogeneic red blood cells in cardiac or other surgery. In certain embodiments, anti-OX40L antibodies may be used to treat a traumatic injury to a limb or joint, such as traumatic knee injury. In certain embodiments, anti-OX40L antibodies may be used to treat diseases including, but not limited to, multiple sclerosis; Behcet's syndrome; Sjogren's syndrome; autoimmune hemolytic anemia; beta thalassemia; amyotrophic lateral sclerosis (Lou Gehrig's Disease); Parkinson's disease; and tenosynovitis of unknown cause, as well as various autoimmune disorders or diseases associated with hereditary deficiencies, including x-linked mental retardation.

In certain embodiments, anti-OX40L antibodies may be used to treat central nervous system (CNS) injuries, including, but not limited to, the effects of neurotoxic neurotransmitters discharged during excitation of inflammation in the central nervous system and to inhibit or prevent the development of glial scars at sites of central nervous system injury. In certain embodiments, anti-OX40L antibodies may be used to treat temporal lobe epilepsy. In connection with epilepsy and the treatment of seizures, reducing the severity and number of recurring seizures, and reducing the severity of the deleterious effects of seizures. In certain embodiments, anti-OX40L antibodies may be used to treat neuronal loss, neuronal degeneration, and gliosis associated with seizures.

In certain embodiments, anti-OX40L antibodies may be used to treat critical illness polyneuropathy and myopathy (CIPNM) acute polyneuropathy; anorexia nervosa; Bell's palsy; chronic fatigue syndrome; transmissible dementia, including Creutzfeld-Jacob disease; demyelinating neuropathy; Guillain-Barre syndrome; vertebral disc disease; Gulf war syndrome; chronic inflammatory demyelinating polyneuropathy, myasthenia gravis; silent cerebral ischemia; sleep disorders, including narcolepsy and sleep apnea; chronic neuronal degeneration; and stroke, including cerebral ischemic diseases. In certain embodiments, anti-OX40L antibodies may be used to treat anorexia and/or anorexic conditions, peritonitis, endotoxemia and septic shock, granuloma formation, heat stroke, Churg-Strauss syndrome, chronic inflammation following acute infections such as tuberculosis and leprosy, systemic sclerosis and hypertrophic scarring.

In certain embodiments, anti-OX40L antibodies may be used to treat the toxicity associated with antibody therapies, chemotherapy, radiation therapy and the effects of other apoptosis inducing agents, e.g. TRAEL and TRADE, and therapies that target IL-1 producing cells, OX40L producing cells, or illicit an inflammatory response. Monoclonal antibody therapies, chemotherapies and other apoptosis inducing therapies that target OX40L cells induce the production and/or release of OX40L. In certain embodiments, by administering therapies that inhibit the effects of OX40L by interfering with its interaction with its receptor and/or receptor accessory, the proinflammatory effects and medical conditions associated with OX40L may be reduced or eliminated.

In certain embodiments, anti-OX40L antibodies may be used to treat non-human animals, such as pets (dogs, cats, birds, primates, etc.), domestic farm animals (horses cattle, sheep, pigs, birds, etc.), or any animal that suffers from an OX40/OX40L inflammatory or arthritic condition. In certain such instances, an appropriate dose may be determined according to the animal's body weight. For example, in certain embodiments, a dose of 0.2-1 mg/kg may be used. In certain embodiments, the dose may be determined according to the animal's surface area, an exemplary dose ranging from 0.1 to 20 mg/in², or from 5 to 12 mg/m². For small animals, such as dogs or cats, in certain embodiments, a suitable dose is 0.4 mg/kg. In certain embodiments, anti-OX40L antibodies are administered by injection or other suitable route one or more times per week until the animal's condition is improved, or it may be administered indefinitely.

In certain embodiments, anti-OX40L antibodies may be used to treat psoriatic lesions. In certain embodiments, anti-OX40L antibodies may be used to treat psoriatic lesions that occur in patients who have ordinary psoriasis or psoriatic arthritis.

In certain embodiments, patients are defined as having ordinary psoriasis if they lack the more serious symptoms of psoriatic arthritis (e.g., distal interphalangeal joint DIP involvement, enthesopathy, spondylitis and dactylitis), but exhibit one of the following: 1) inflamed swollen skin lesions covered with silvery white scale (plaque psoriasis or psoriasis vulgaris); 2) small red dots appearing on the trunk, arms or legs (guttate psoriasis); 3) smooth inflamed lesions without scaling in the flexural surfaces of the skin (inverse psoriasis); 4) widespread reddening and exfoliation of fine scales, with or without itching and swelling (erythrodermic psoriasis); 5) blister-like lesions (pustular psoriasis); 6) elevated inflamed scalp lesions covered by silvery white scales (scalp psoriasis); 7) pitted fingernails, with or without yellowish discoloration, crumbling nails, or inflammation and detachment of the nail from the nail bed (nail psoriasis).

In treating ordinary psoriasis, in certain embodiments, anti-OX40L antibodies may be administered in an amount and for a time sufficient to induce an improvement in the patient's condition as measured according to any indicator that reflects the severity of the patient's psoriatic lesions. In certain embodiments, one or more such indicators may be assessed for determining whether the amount of anti-OX40L antibody and duration of treatment is sufficient. In certain embodiments, the anti-OX40L antibody is administered in an amount and for a time sufficient to induce an improvement over baseline in either the psoriasis area and severity index (PASI) or the Target Lesion Assessment Score. In certain embodiments, both indicators are used. In certain embodiments, when PASI score is used as the indicator, treatment is regarded as sufficient when the patient exhibits an at least 50% improvement in his or her PASI score, or alternatively, when the patient exhibits an at least 75% improvement in PASI score. In certain embodiments, using the Psoriasis Target Lesion Assessment Score to measure sufficiency of treatment involves determining for an individual psoriatic lesion whether improvement has occurred in one or more of the following, each of which is separately scored: plaque elevation; amount and degree of scaling or degree of erythema; and target lesion response to treatment. In certain embodiments, a Psoriasis Target Lesion Assessment Score is determined by adding together the separate scores for all four of the aforementioned indicia, and determining the extent of improvement by comparing the baseline score to the score after treatment has been administered.

In certain embodiments, a satisfactory degree of improvement in psoriasis patients is obtained by administering the anti-OX40L antibodies one or more times per week. In certain embodiments, the anti-OX40L antibodies may be administered one time, two times or three or more times per week. In certain embodiments, treatment may be continued over a period of at least one week, for two weeks, three weeks, four weeks or longer. In certain embodiments, treatment may be discontinued after the patient improves, then resumed if symptoms return, or alternatively, the treatment may be administered continuously for an indefinite period. In certain embodiments, the route of administration is subcutaneous injection. In certain embodiments, anti-OX40L antibodies are administered by injection at a dose 5-12 mg/m², or a flat dose of either 25 mg or 50 mg. In certain embodiments, a dose of 25 mg is injected two times per week, and in certain embodiments, a dose of 50 mg is injected one time per week. In certain embodiments, anti-OX40L antibodies are administered once every 6 months. In certain embodiments, anti-OX40L antibodies are administered once every 3 months. In certain embodiments, anti-OX40L antibodies are administered once every month. In certain embodiments of treating pediatric psoriasis patients, the dose administered by injection is 0.1 mg/kg, up to a maximum dose of 25 mg.

In certain embodiments, anti-OX40L antibodies may be used to treat ordinary psoriasis in combination with one, two, three or more other medications that are effective against psoriasis. These additional medications may be administered before, simultaneously with, or sequentially with anti-OX40L antibodies. Exemplary drugs suitable for combination therapies of psoriasis include, but are not limited to, pain medications (analgesics), including but not limited to acetaminophen, codeine, propoxyphene napsylate, oxycodone hydrochloride, hydrocodone 24 bitartrate and tramadol. In certain embodiments, an anti-OX40L antibody with our without ENBREL™, may be administered in combination with methotrexate, sulfasalazine, gold salts, azathioprine, cyclosporine, antimalarials, oral steroids (e.g., prednisone) or colchicine. Non-steroidal anti-inflammatories may also be coadministered with an anti-OX40L antibody and TNFR mimic, including but not limited to: salicylic acid (aspirin); ibuprofen; indomethacin; celecoxib; rofecoxib; ketorolac; nambumetone; piroxicam; naproxen; oxaprozin; sulindac; ketoprofen; diclofenac; and other COX-1 and COX-2 inhibitors, salicylic acid derivatives, propionic acid derivatives, acetic acid derivatives, fumaric acid derivatives, carboxylic acid derivatives, butyric acid derivatives, oxicarns, pyrazoles and pyrazolones, including newly developed anti-inflammatories.

In certain embodiments, anti-OX40L antibodies may be used to treat psoriasis in combination with one or more of the following: topical steroids, systemic steroids, antagonists of inflammatory cytokines, antibodies against T cell surface proteins, anthralin, coal tar, vitamin D3 and its analogs (including 1,25-dihydroxy vitamin D3 and calcipotriene), topical retinoids, oral retinoids (including but not limited to etretinate, acitretin and isotretinoin), topical salicylic acid, methotrexate, cyclosporine, hydroxyurea, and/or sulfasalazine. In certain embodiments, anti-OX40L antibodies may be administered in combination with one or more of the following compounds: minocycline; misoprostol; oral collagen; penicillamine; 6-mercaptopurine; nitrogen mustard; gabapentin; bromocriptine; somatostatin; peptide T; anti-CD4 monoclonal antibody; fumaric acid; polyunsaturated ethyl ester lipids; zinc; and/or other drugs that may be used to treat psoriasis.

In certain embodiments, anti-OX40L antibodies may be used to treat psoriasis by administering anti-OX40L antibodies in combination with one or more of the following topically applied compounds: oils, including fish oils, nut oils and vegetable oils; aloe vera; jojoba; Dead Sea salts; capsaicin; milk thistle; witch hazel; moisturizers; and/or Epsom salts.

In certain embodiments, anti-OX40L antibodies may be used to treat psoriasis by administering anti-OX40L antibodies in combination with one or more of the following exemplary therapies: plasmapheresis; phototherapy with ultraviolet light B; psoralen combined with ultraviolet light A (PUVA); and/or sunbathing.

In certain embodiments, anti-OX40L antibodies may be used to treat lung disorders including, but not limited to, asthma, chronic obstructive pulmonary disease, pulmonary alveolar proteinosis, bleomycin-induced pneumopathy and fibrosis, radiation-induced pulmonary fibrosis, cystic fibrosis, collagen accumulation in the lungs, and ARDS. In certain embodiments, such diseases may be treated with combinations anti-OX40L antibodies and an IL-4 inhibitor. In certain embodiments, anti-OX40L antibodies may be used to treat various skin disorders, including but not limited to dermatitis herpetiformis (Duhring's disease), atopic dermatitis, contact dermatitis, urticaria (including chronic idiopathic urticaria), and autoimmune blistering diseases, including pemphigus vulgaris and bullous pemphigoid. In certain embodiments, anti-OX40L antibodies may be used to treat myesthenia gravis, sarcoidosis, including pulmonary sarcoidosis, scleroderma, reactive arthritis, hyper IgE syndrome, multiple sclerosis and idiopathic hypereosinophil syndrome. In certain embodiments, anti-OX40L antibodies may be used to treat allergic reactions to medication and as an adjuvant to allergy immunotherapy.

In certain embodiments, anti-OX40L antibodies may be used to treat cardiovascular disorders or injuries including, but not limited to, aortic aneurysms; including abdominal aortic aneurysms, acute coronary syndrome, arteritis; vascular occlusion, including cerebral artery occlusion; complications of coronary by-pass surgery; ischemidreperfusion injury; heart disease, including atherosclerotic heart disease, myocarditis, including chronic autoimmune myocarditis and viral myocarditis; heart failure, including chronic heart failure, congestive heart failure, cachexia of heart failure; myocardial infarction; restenosis and/or atherosclerosis after heart surgery or after carotid artery balloon angioplastic procedures; silent myocardial ischemia; left ventricular pump dysfunction, post implantation complications of left ventricular assist devices; Raynaud's phenomena; thrombophlebitis; vasculitis, including Kawasaki's vasculitis; veno-occlusive disease, giant cell arteritis, Wegener's granulomatosis; mental confusion following cardio pulmonary by pass surgery, and Schoenlein-Henoch purpura. In certain embodiments, combinations of anti-OX40L antibodies, TNF inhibitors and angiogenesis inhibitors (e.g. anti-VEGF) may be used to treat certain cardiovascular diseases such as aortic aneurysms and tumors.

It is understood that the response by individual patients to the aforementioned medications or combination therapies may vary, and an appropriate efficacious combination of drugs for each patient may be determined by his or her physician.

The cynomolgus monkey provides a useful model for certain diseases. Exemplary diseases include, but are not limited to, transplantation rejection syndrome and inflammatory bowel disease-like disease. When testing the efficacy of a human MAb In cynomolgus monkey human disease model, in certain embodiments, it is useful to determine whether the anti-OX40L MAb binds to OX40L in humans and cynomolgus monkeys at a comparable level.

In certain embodiments, an anti-OX40L antibody may be part of a conjugate molecule comprising all or part of the anti-OX40L antibody and a cytotoxic agent. The term “cytotoxic agent” refers to a substance that inhibits or prevents the function of cells and/or causes the death or destruction of cells. The term includes, but is not limited to, radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof. Cytotoxic agents include, but are not limited to, Adriamycin, Doxorubicin, 5-Fluorouracil, Cytosine arabinoside (“Ara-C”), Cyclophosphamide, Thiotepa, Taxotere (docetaxel), Busulfan, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Carminomycin, Aminopterin, Dactinomycin, Mitomycins, Esperamicins, Melphalan and other related nitrogen mustards.

In certain embodiments, an anti-OX40L antibody may be part of a conjugate molecule comprising all or part of the anti-OX40L antibody and a prodrug. In certain embodiments, the term “prodrug” refers to a precursor or derivative form of a pharmaceutically active substance. In certain embodiments, a prodrug is less cytotoxic to cells compared to the parent drug and is capable of being enzymatically activated or converted into the more active cytotoxic parent form. Exemplary prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs and optionally substituted phenylacetamide-containing prodrugs, 5-fluorocytosine and other 5-fluorouridine prodrugs which can be converted into a more active cytotoxic free drug. Examples of cytotoxic drugs that can be derivatized into a prodrug form include, but are not limited to, those cytotoxic agents described above. See, e.g., U.S. Pat. No. 6,702,705.

In certain embodiments, antibody conjugates function by having the antibody portion of the molecule target the cytotoxic portion or prodrug portion of the molecule to a specific population of cells in the patient. In the case of anti-OX40L antibodies, such conjugate molecules may be used, for example, in certain embodiments, to destroy APCs that express OX40L at sites of abnormal or destructive inflammatory responses.

In certain embodiments, methods of treating a patient comprising administering a therapeutically effective amount of an anti-OX40L antibody are provided. In certain embodiments, methods of treating a patient comprising administering a therapeutically effective amount of an antibody conjugate are provided. In certain embodiments, an antibody is used in conjunction with a therapeutically effective amount of at least one additional therapeutic agent. Exemplary therapeutic agents include, but are not limited to, the bone morphogenic factors designated BMP-1 through BMP-12; transforming growth factor-β (TGF-β) and TGF-β family members; interleukin-1 (IL-1) inhibitors, including, but not limited to, IL-1Ra and derivatives thereof and Kineret™; TNFα inhibitors, including, but not limited to, soluble TNFα receptors, ENBREL™, anti-TNFα antibodies, Remicade™, and D2E7 antibodies; parathyroid hormone and analogs thereof; parathyroid related protein and analogs thereof; E series prostaglandins; bisphosphonates (such as alendronate and others); bone-enhancing minerals such as fluoride and calcium; non-steroidal anti-inflammatory drugs (NSAIDs), including, but not limited to, COX-2 inhibitors, such as Celebrex™ and Vioxx™; immunosuppressants, such as methotrexate or leflunomide; serine protease inhibitors, including, but not limited to, secretory leukocyte protease inhibitor (SLPI); IL-6 inhibitors (including, but not limited to, antibodies to IL-6), IL-8 inhibitors (including, but not limited to, antibodies to IL-8); IL-18 inhibitors (including, but not limited to, IL-18 binding protein and IL-18 antibodies); Interleukin-1 converting enzyme (ICE) modulators; fibroblast growth factors FGF-1 to FGF-10 and FGF modulators; PAF antagonists; keratinocyte growth factor (KGF), KGF-related molecules, and KGF modulators; matrix metalloproteinase (MMP) modulators; Nitric oxide synthase (NOS) modulators, including, but not limited to, modulators of inducible NOS; modulators of glucocorticoid receptor; modulators of glutamate receptor; modulators of lipopolysaccharide (LPS) levels; and noradrenaline and modulators and mimetics thereof. See, e.g., Published PCT Application No. WO 03/0002713 for exemplary details on exemplary additional therapeutic agents.

As discussed above, in certain embodiments, anti-OX40L antibodies may be administered concurrently with one or more other drugs that are administered to the same patient, each drug being administered according to a regimen suitable for that medicament. Such treatment encompasses pre-treatment, simultaneous treatment, sequential treatment, and alternating regimens. Additional examples of such drugs include, but are not limited to antivirals, antibiotics, analgesics, corticosteroids, antagonists of inflammatory cytokines, DMARDs, and nonsteroidal anti-inflammatories. Additionally, in certain embodiments, anti-OX40L antibodies are administered in combination with pentoxifylline or thalidomide.

In certain embodiments, various medical disorders are treated with anti-OX40L antibodies in combination with another cytokine or cytokine inhibitor. For example, in certain embodiments, anti-OX40L antibodies may be administered in a composition that also contains a compound that inhibits the interaction of other inflammatory cytokines with their receptors. In certain embodiments, the anti-OX40L antibody and cytokine inhibitors may be administered as separate compositions, and these may be administered by the same or different routes. Examples of cytokine inhibitors used in combination with anti-OX40L antibodies include, but are not limited to, those that antagonize, for example, TGFβ, IFNγ, type II IL-1 receptor, IL-6 or IL-8 and TNF. In certain embodiments, the combination of an anti-OX40L antibody and an IL-1 inhibitor, e.g. type II IL-1 receptor or IL-6 may be used to treat the recurrence of seizures, including seizures induced by GABAA receptor antagonism, seizures associated with EEG ictal episodes and motor limbic seizures occurring during status epilepticus. In certain embodiments, the combination of anti-OX40L antibodies and IFNγ-1b may be used to treat idiopathic pulmonary fibrosis and cystic fibrosis. Other exemplary combinations for treating diseases, such as those described herein, include the use of anti-OX40L antibodies with compounds that interfere with the binding of RANK and RANK-ligand, such as RANK-ligand inhibitors, 8 or soluble forms of RANK, including RANK:Fc. In certain embodiments, the combination of anti-OX40L antibodies and RANK:Fc may be used to inhibit or prevent bone destruction in various settings including but not limited to various rheumatic disorders, osteoporosis, multiple myeloma or other malignancies that cause bone degeneration, or anti-tumor therapy aimed at inhibiting or preventing metastasis to bone, or bone destruction associated with prosthesis wear debris or with periodontitis. In certain embodiments, anti-OX40L antibodies may be administered in combination with one or more of the following: G-CSF, GM-CSF, IL-2 and/or inhibitors of protein kinase A type 1 to enhance T cell proliferation in MV-infected patients who are receiving antiretroviral therapy. In certain embodiments, anti-OX40L antibodies may be administered in combination with one or more of the following: soluble forms of an IL-17 receptor (such as IL-17R:Fc), IL-18 binding protein, soluble forms of IL-18 receptors, and IL-18 antibodies, antibodies against IL-18 receptors or antibodies against CD30-ligand and/or against CD4.

In certain embodiments, medical disorders may be treated with a combination of anti-OX40L antibodies, a TNF inhibitor (e.g., TNFR:Fc (ENBREL™ marketed for clinical uses by Immunex Corp)) and any combination of the above described cytokines or cytokine inhibitors that are active agents in combination therapies. In certain embodiments, combination therapy methods for treating rheumatoid arthritis, stroke, and congestive heart failure, include administering anti-OX40L antibodies and ENBREL™. In certain embodiments, anti-OX40L antibodies and TNF inhibitors may be used in combination therapies for use in medicine and in particular in therapeutic and preventive therapies for medical disorders such as those described herein. In certain embodiments, the use in medicine may involve the treatment of any of the medical disorders as described herein with a combination therapy that includes administering a combination of anti-OX40L antibodies and ENBREL™. In certain embodiments, the anti-OX40L antibodies and TNF inhibitor (ENBREL™) may be in the form of compounds, compositions or combination therapies. Where the compounds are used together with one or more other components, the compound and the one or more other components may be administered simultaneously, separately or sequentially (e.g., in a pharmaceutical format).

Exemplary TNF antagonists that may be used with anti-OX40L antibodies include, but are not limited to, peptide fragments of TNF antisense oligonucleotides or ribozymes that inhibit TNFα, production, antibodies directed against TNFα (i.e. REMICADE), and recombinant proteins comprising all or portions of receptors for TNFα or modified variants thereof, including, but not limited to, genetically-modified muteins, multimeric forms and sustained-release formulations. Exemplary TNFα inhibitors are disclosed in U.S. Pat. Nos. 5,641,751 and 5,519,000, and the D-amino acid-containing peptides are described in U.S. Pat. No. 5,753,628.

Exemplary compounds that are TNF inhibitors that may be used in combination therapies include, but are not limited to, small molecules such as thalidomide or thalidomide analogs, pentoxifylline, or matrix metalloproteinase (MMP) inhibitors and other small molecules. Exemplary MMP inhibitors include, for example, those described in U.S. Pat. Nos. 5,883,131; 5,863,949; and 5,861,510, as well as the mercapto alkyl peptidyl compounds described in U.S. Pat. No. 5,872,146. Other small molecules capable of reducing TNFα production, include, for example, the molecules described in U.S. Pat. Nos. 5,508,300; 5,596,013; and 5,563,143, any of which can be administered in combination with TNFα inhibitors such as soluble TNFRs or antibodies against TNFα. Additional exemplary small molecules useful for treating the TNFα-mediated diseases described herein include the MMP inhibitors that are described in U.S. Pat. Nos. 5,747,514 and 5,691,382, as well as the hydroxamic acid derivatives described in U.S. Pat. No. 5,821,262. The diseases described herein also may be treated with small molecules that inhibit phosphodiesterase IV and TNFα production, such as substituted oxime derivatives (WO 96/00215), quinoline sulfonamides (U.S. Pat. No. 5,834,485), aryl furan derivatives (WO 99/18095) and heterobicyclic derivatives (WO 96/01825; GB 2 291422 A). In certain embodiments, thiazole derivatives that suppress TNFα and IFNγ (WO 99/15524), as well as xanthine derivatives that suppress TNFα and other proinflammatory cytokines (see, for example, U.S. Pat. Nos. 5,118,500; 5,096,906; and 5,196,430) may also be useful for treatment of the diseases described herein. Additional exemplary small molecules to treat the conditions described herein include those disclosed in U.S. Pat. No. 5,547,979.

In certain embodiments, antisense oligonucleotides for suitable for treating diseases in therapeutic combinations include, for example, the anti-TNFα oligonucleotides described in U.S. Pat. No. 6,080,580, which proposes the use of such oligonucleotides as candidates for testing in animal models of diabetes mellitus, rheumatoid arthritis, contact sensitivity, Crohn's disease, multiple sclerosis, pancreatitis, hepatitis, and heart transplant.

In certain embodiments, combination therapies utilize soluble TNFRs as a TNFα antagonist. Soluble forms of TNFRs may include monomers, fusion proteins (also called “chimeric proteins”), dimers, trimers or higher order multimers. In certain embodiments, the soluble TNFR derivative is one that mimics the 75 kDa TNFR or the 55 kDa TNFR and that binds to TNFα in the patient's body. In certain embodiments, these soluble TNFR mimics may be derived from TNFRs p55 or p75 or fragments thereof.

In certain embodiments, TNFRs other than p55 and p75 may be used for deriving soluble compounds for treating the various medical disorders described herein, for example, the TNFR that is described in WO 99/04001. Exemplary soluble TNFR molecules used to construct TNFR mimics include, but are not limited to, analogs or fragments of native TN FRs having at least 20 amino acids, that lack the transmembrane region of the native TNFR, and that are capable of binding TNFα.

In certain embodiments, antagonists derived from TNFRs compete for TNFα with the receptors on the cell surface, thus inhibiting TNFα from binding to cells, thereby preventing it from manifesting its biological activities. Binding of soluble TNFRs to TNFα or LTα can be assayed using ELISA or any other convenient assay. In certain embodiments, soluble TNFα receptors are used in the manufacture of medicaments for the treatment of numerous diseases.

In certain embodiments, anti-OX40L antibodies may be administered to a patient in a therapeutically effective amount along with therapeutically effective amounts of an IL-4 inhibitor, and optionally, a TNFα inhibitor, e.g. ENBREL™ in any of the aforementioned combination therapies.

IL-4 antagonists that may be employed according to certain embodiments include, but are not limited to, IL-4 receptors (IL-4R) and other IL-4-binding molecules, IL-4 muteins and antibodies that bind specifically with IL-4 or IL-4 receptors thereby blocking signal transduction, as well as antisense oligonucleotides and ribozymes targeted to IL-4 or IL-4R. Antibodies specific for IL-4 or IL-4 receptor may be prepared using standard procedures. In certain embodiments, IL-4 receptors suitable for use as described herein are soluble fragments of human IL-4R that retain the ability to bind IL-4. In certain embodiments, such fragments are capable of binding IL-4, and retain all or part of the IL-4R extracellular region.

Exemplary IL-4 antagonists that may be useful in combination therapies include molecules that selectively block the synthesis of endogenous IL-4 or IL-4R. Exemplary IL-4 receptors are described in U.S. Pat. No. 5,599,905; Idzerda et al., J. Exp. Med. 171:861-873, March 1990 (human IL-4R); and Mosley et al., Cell 59:335-348, 1989 (murine IL-4R). The protein described in those three references is sometimes referred to in the scientific literature as IL-4Rα.

In certain embodiments, in view of the disease to be treated and the desired level of treatment, two, three, or more agents may be administered. In certain embodiments, such agents may be provided together by inclusion in the same formulation. In certain embodiments, such agents and an antibody may be provided together by inclusion in the same formulation. In certain embodiments, such agents may be provided together by inclusion in a treatment kit. In certain embodiments, such agents may be provided separately. In certain embodiments, when administered by gene therapy, the genes encoding protein agents and/or an antibody may be included in the same vector. In certain embodiments, the genes encoding protein agents and/or an antibody may be under the control of the same promoter region. In certain embodiments, the genes encoding protein agents and/or an antibody may be in separate vectors.

In certain embodiments, the invention provides for pharmaceutical compositions are provided comprising a therapeutically effective amount of an antibody together with a pharmaceutically acceptable diluent, carrier, solubilizer, emulsifier, preservative and/or adjuvant.

In certain embodiments, the invention provides for pharmaceutical compositions are provided comprising a therapeutically effective amount of an antibody and a therapeutically effective amount of at least one additional therapeutic agents, together with a pharmaceutically acceptable diluent, carrier, solubilizer, emulsifier, preservative and/or adjuvant.

In certain embodiments, acceptable formulation materials preferably are nontoxic to recipients at the dosages and concentrations employed.

In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18^{th} Edition, A. R. Gennaro, ed., Mack Publishing Company (1990).

In certain embodiments, an antibody and/or an additional therapeutic molecule is linked to a half-life extending vehicle known in the art. Such vehicles include, but are not limited to, the Fc domain, polyethylene glycol, and dextran. Such vehicles are described, e.g., in U.S. application Ser. No. 09/428,082 and published PCT Application No. WO 99/25044.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies.

In certain embodiments, the primary vehicle or carrier in a pharmaceutical composition may be either aqueous or non-aqueous in nature. For example, in certain embodiments, a suitable vehicle or carrier may be water for injection, physiological saline solution or artificial cerebrospinal fluid, possibly supplemented with other materials common in compositions for parenteral administration. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which may further include sorbitol or a suitable substitute therefor. In certain embodiments, a pharmaceutical composition is an aqueous or liquid formulation comprising an acetate buffer of about pH 4.0-5.5, a polyol (polyalcohol), and optionally, a surfactant, wherein the composition does not comprise a salt, e.g., sodium chloride, and wherein the composition is isotonic for the patient. Exemplary polyols include, but are not limited to, sucrose, glucose, sorbitol, and mannitol. An exemplary surfactant includes, but is not limited to, polysorbate. In certain embodiments, a pharmaceutical composition is an aqueous or liquid formulation comprising an acetate buffer of about pH 5.0, sorbitol, and a polysorbate, wherein the composition does not comprise a salt, e.g., sodium chloride, and wherein the composition is isotonic for the patient. Certain exemplary compositions are found, for example, in U.S. Pat. No. 6,171,586. Additional pharmaceutical carriers include, but are not limited to, oils, including petroleum oil, animal oil, vegetable oil, peanut oil, soybean oil, mineral oil, sesame oil, and the like. Aqueous dextrose and glycerol solutions can also be employed as liquid carriers, particularly for injectable solutions. In certain embodiments, a composition comprising an antibody, with or without at least one additional therapeutic agents, may be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising an antibody, with or without at least one additional therapeutic agents, may be formulated as a lyophilizate using appropriate excipient solutions (e.g., sucrose) as diluents.

In certain embodiments, anti-OX40L antibodies are administered in the form of a physiologically acceptable composition comprising purified recombinant protein in conjunction with physiologically acceptable carriers, excipients or diluents. In certain embodiments, such carriers are nontoxic to recipients at the dosages and concentrations employed. In certain embodiments, preparing such compositions may involve combining the anti-OX40L antibodies with buffers, antioxidants such as ascorbic acid, low molecular weight polypeptides (such as those having fewer than 10 amino acids), proteins, amino acids, carbohydrates such as glucose, sucrose or dextrins, chelating agents such as EDTA, glutathione and/or other stabilizers and excipients. In certain embodiments, appropriate dosages are determined in standard dosing trials, and may vary according to the chosen route of administration. In certain embodiments, in accordance with appropriate industry standards, preservatives may also be added, which include, but are not limited to, benzyl alcohol. In certain embodiments, the amount and frequency of administration may be determined based on such factors as the nature and severity of the disease being treated, the desired response, the age and condition of the patient, and so forth.

In certain embodiments, pharmaceutical compositions can be selected for parenteral delivery. The preparation of such pharmaceutically acceptable compositions is within the skill of the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition may be in the form of a pyrogen-free, parenterally acceptable aqueous solution comprising the desired antibody, with or without additional therapeutic agents, in a pharmaceutically acceptable vehicle. In certain embodiments, a vehicle for parenteral injection is sterile distilled water in which the antibody, with or without at least one additional therapeutic agent, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide for the controlled or sustained release of the product which may then be delivered via a depot injection. In certain embodiments, hyaluronic acid may also be used, and may have the effect of promoting sustained duration in the circulation. In certain embodiments, implantable drug delivery devices may be used to introduce the desired molecule.

In certain embodiments, a pharmaceutical composition may be formulated for inhalation. In certain embodiments, an antibody, with or without at least one additional therapeutic agent, may be formulated as a dry powder for inhalation. In certain embodiments, an inhalation solution comprising an antibody, with or without at least one additional therapeutic agent, may be formulated with a propellant for aerosol delivery. In certain embodiments, solutions may be nebulized. Pulmonary administration is further described in PCT application no. PCT/US94/001875, which describes pulmonary delivery of chemically modified proteins.

In certain embodiments, it is contemplated that formulations may be administered orally. In certain embodiments, an antibody, with or without at least one additional therapeutic agents, that is administered in this fashion may be formulated with or without those carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized. In certain embodiments, at least one additional agent can be included to facilitate absorption of the antibody and/or any additional therapeutic agents. In certain embodiments, diluents, flavorings, low melting point waxes, vegetable oils, lubricants, suspending agents, tablet disintegrating agents, and binders may also be employed.

In certain embodiments, a pharmaceutical composition may involve an effective quantity of antibodies, with or without at least one additional therapeutic agents, in a mixture with non-toxic excipients which are suitable for the manufacture of tablets. In certain embodiments, by dissolving the tablets in sterile water, or another appropriate vehicle, solutions may be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving antibodies, with or without at least one additional therapeutic agents, in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations may include semipermeable polymer matrices in the form of shaped articles, e.g. films, or microcapsules. Sustained release matrices may include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions may also include liposomes, which can be prepared by any of several methods known in the art. See e.g., Eppstein et al., Proc. Nat. Acad. Sci. USA, 82:3688-3692 (1985); EP 036,676; EP 088,046 and EP 143,949.

In certain embodiments, the pharmaceutical composition to be used for in vivo administration is sterile. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, after the pharmaceutical composition has been formulated, it may be stored in sterile vials as a solution, suspension, gel, emulsion, solid, or as a dehydrated or lyophilized powder. In certain embodiments, such formulations may be stored either in a ready-to-use form or in a form (e.g., lyophilized) that is reconstituted prior to administration.

In certain embodiments, the present invention is directed to kits for producing a single-dose administration unit. In certain embodiments, the kits may each contain both a first container having a dried protein and a second container having an aqueous formulation. In certain embodiments of this invention, kits containing single and multi-chambered pre-filled syringes (e.g., liquid syringes and lyosyringes) are included.

In certain embodiments, the effective amount of a pharmaceutical composition comprising an antibody, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which the antibody, with or without at least one additional therapeutic agent, is being used, the route of administration, and the size (body weight, body surface or organ size) and/or condition (the age and general health) of the patient. In certain embodiments, the clinician may titer the dosage and modify the route of administration to obtain the optimal therapeutic effect. In certain embodiments, a typical dosage may range from about 0.1 μg/kg to up to about 100 mg/kg or more, depending on the factors mentioned above. In certain embodiments, the dosage may range from 0.1 μg/kg up to about 100 mg/kg; or 1 μg/kg up to about 100 mg/kg; or 5 μg/kg up to about 100 mg/kg.

In certain embodiments, the frequency of dosing will take into account the pharmacokinetic parameters of the antibody and/or any additional therapeutic agents in the formulation used. In certain embodiments, a clinician will administer the composition until a dosage is reached that achieves the desired effect. In certain embodiments, the composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages may be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

As discussed above, in various embodiments, any efficacious route of administration may be used to administer anti-OX40L antibodies. If injected, in certain embodiments, anti-OX40L antibodies may be administered, for example, via intra-articular, intravenous, intramuscular, intralesional, intraperitoneal, intracranial, inhalation or subcutaneous routes by bolus injection or by continuous infusion. In certain embodiments, pulmonary diseases can involve intranasal and inhalation methods of delivery. Exemplary methods of administration include, but are not limited to, sustained release from implants, aerosol inhalation, eyedrops, oral preparations, including pills, syrups, lozenges or chewing gum, and topical preparations such as lotions, gels, sprays, ointments or other suitable techniques.

In certain embodiments, administration by inhalation is beneficial when treating diseases associated with pulmonary disorders. In certain embodiments, anti-OX40L antibodies may be administered by implanting cultured cells that express the antibodies. In certain embodiments, the patient's own cells are induced to produce by transfection in vivo or ex vivo with one or more vectors that encode an anti-OX40L antibody. In certain embodiments, this vector can be introduced into the patient's cells, for example, by injecting naked DNA or liposome-encapsulated DNA that encodes an anti-OX40L antibody, or by other methods of transfection. When anti-OX40L antibodies are administered in combination with one or more other biologically active compounds, in certain embodiments, these may be administered by the same or by different routes, and may be administered simultaneously, separately or sequentially.

In certain embodiments, the composition may be administered locally via implantation of a membrane, sponge or another appropriate material onto which the desired molecule has been absorbed or encapsulated. In certain embodiments, where an implantation device is used, the device may be implanted into any suitable tissue or organ, and delivery of the desired molecule may be via diffusion, timed-release bolus, or continuous administration.

In certain embodiments, it may be desirable to use a pharmaceutical composition comprising an antibody, with or without at least one additional therapeutic agent, in an ex vivo manner. In such instances, cells, tissues and/or organs that have been removed from the patient are exposed to a pharmaceutical composition comprising an antibody, with or without at least one additional therapeutic agent, after which the cells, tissues and/or organs are subsequently implanted back into the patient.

In certain embodiments, an antibody and/or any additional therapeutic agents can be delivered by implanting certain cells that have been genetically engineered, using methods such as those described herein, to express and secrete the polypeptides. In certain embodiments, such cells may be animal or human cells, and may be autologous, heterologous, or xenogeneic. In certain embodiments, the cells may be immortalized. In certain embodiments, in order to decrease the chance of an immunological response, the cells may be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### EXAMPLES

### Example 1

### Production of Certain Human Monoclonal Antibodies

Certain human anti-OX40L monoclonal antibodies are produced in transgenic mice expressing human immunoglobulin genes. Mice are given 8 injections overall. On day 0, 10⁷ CHO cells expressing human OX40L are injected into footpads of the transgenic mice. On days 3, 7, 10, and 14, the mice are given boosting injections, each injection containing 10⁷ CHO cells expressing human OX40L plus 10 μg of a CpG polynucleotide. On days 17, 21, and 27, the mice are given additional boosting injections containing OX40L-Flag fusion protein. Whole blood from the immunized transgenic mice is harvested on day 31 and hybridomas are prepared via standard techniques. The resulting hybridoma supernatants are screened by FMAT and ELISA for antibody binding to OX40L. In the FMAT assay, plates are coated with cells expressing OX40L, hybridoma supernatant is added, and a secondary anti-human Ig antibody is then added for detection via standard ELISA techniques. Negative controls are the corresponding, non-transfected cells that do not express OX40L. The ELISA assay is done in a similar way, except the plates are directly coated with OX40L.

Fc fusions proteins are used in a BIACore method to screen the resulting antibodies. Human Fc-OX40L is a fusion protein comprised of the Fc domain of human IgG fused to human OX40L and hOX40R-Fc is comprised of the human IgG Fc domain fused to the human OX40 receptor. These fusion proteins are made by transiently transfecting 293T or COS PKB adherent cells grown and maintained in DMEM supplemented with 5% FBS+1× Non-Essential Amino Acids+1× Pen Strep Glut+1× Sodium Pyruvate.

Approximately, 4-5×10⁷ 293T cells (i.e., ATCC CRL-11268) are seeded in a 850 cm² roller bottle overnight. The previously seeded cells are then transfected the following day using FuGene6 transfection reagent. A DNA-FuGene6 mixture is prepared in approximately 6.75 mL serum-free DMEM, by first adding 675 μl FuGene6 transfection reagent to the DMEM, followed by adding 112.5 μg of plasmid DNA encoding the Fc fusion protein. The mixture is incubated at room temperature for 30 minutes. The entire mixture is then added to a roller bottle. The roller bottle is gassed with a 5% CO₂ gas mixture, capped tightly, and placed in a 37° C. incubator on a roller rack rotating at 0.35 RPM. The transfection is performed for 24 hours after which the medium is replaced with 100 mL DMEM+1× Insulin-Transferrin-Selenium Supplement+1× Pen Strep Glu+1× Non-Essential Amino Acids+1× Sodium Pyruvate and resulted in cells constituatively expressing the Fc fusion proteins. Two 100 ml 5 day harvests are obtained from each roller bottle. The harvested serum-free conditioned medium is pooled together and centrifuged at 4,000 RPM for 30 minutes at 4° C. before purification of the Fc fusion proteins.

Approximately, 2×10⁷ COS cells (i.e., ATCC CRL-1650) are seeded in a 850 cm² roller bottles overnight. The previously seeded cells are then transfected the following day using FuGene6 transfection reagent. A DNA-FuGene6 mixture is prepared in approximately 7.25 mL serum-free DMEM, by first adding 241.5 μl FuGene6 transfection reagent to the DMEM, followed by adding 120.75 μg of plasmid DNA encoding the Fc fusion protein. The mixture is incubated at room temperature for 30 minutes. The entire mixture is then added to a roller bottle. The roller bottle is gassed with a 5% CO₂ gas mixture, capped tightly, and placed in a 37° C. incubator on a roller rack rotating at 0.35 RPM. The transfection is performed for 24 hours after which the medium is replaced with 100 mL DMEM+1× Insulin-Transferrin-Selenium Supplement+1× Pen Strep Glu+1× Non-Essential Amino Acids+1× Sodium Pyruvate. Two 250 ml 5 day harvests are obtained from each roller bottle. The harvested serum-free conditioned medium is pooled together and centrifuged at 4,000 RPM for 30 minutes at 4° C. before purification of the Fc fusion proteins.

The antibodies discussed above are screened for their ability to bind human OX40L using a BIACore microchip analysis. Specifically, a BIACore 2000 analyzer is used in concert with a CM5 sensor chip (BIACore; Piscataway, N.J.). HFc-OX40L fusion protein is immobilized to the sensor chip surface according to manufacturer's instructions, using a continuous flow of HBS-EP buffer (10 mM HEPES, 0.15M NaCl, 3.4 mM EDTA, 0.005% P-20, pH 7.4). Carboxyl groups on the sensor chip surfaces are activated by injecting 60 μL of a mixture containing 0.2 M N-ethyl-N′-(dimethylaminopropyl)carbodiimide (EDC) and 0.05 M N-hydroxysuccinimide (NHS). Specific surfaces are obtained by injecting recombinant hFc-OX40L diluted in 10 mM acetate, pH 4.5 (BIACore, Inc.; Piscataway, N.J.) at a concentrations of 10 μg/ml to obtain a moderate surface density of 2,000 resonance units (RU). In certain embodiments, other concentrations of hFc-OX40L, such as 25 μg/ml, may also be used.

Excess reactive groups on the chip surfaces are deactivated by injecting 60 μL of 1 M ethanolamine. A blank, mock-coupled reference surface is also prepared on each sensor chip. For mock-coupling, activation and inactivation steps are carried out without protein.

Monoclonal antibody candidates are diluted into sample buffer (1×PBS+0.005% P-20+0.1 mg/ml BSA (fraction V, IgG free; Sigma, Inc.) filtered and degassed) to a concentration of 25 nM and injected over the hFc-OX40L surface for two minutes at a flow rate of 80 μL/min. A separate hFc-OX40R control is diluted into sample buffer (filtered and degassed) to a concentration of 50 nM and injected over the hFc-OX40L surface for two minutes at a flow rate of 80 μL/min. For all analyses, the instrument running buffer is 1×PBS (no calcium chloride, no magnesium chloride; Gibco Inc.)+0.005% P-20 (filtered and degassed), and the temperature is set to 25° C. After a dissociation time of 5 minutes, the surface is regenerated by injecting 8 mM glycine, pH 3.0 (BIACore, Inc.; Piscataway, N.J.), 1 M NaCl for 30 seconds. Binding curves are compared qualitatively for binding signal intensity, as well as for dissociation rates. Antibodies that demonstrate a positive binding signal are chosen for further study.

Hundreds of positive clones are identified according to the above screening method. Ten exemplary human monoclonal antibodies are selected for further study (Ab A through Ab J). Table 2 provides the EC₅₀ values for eight of these antibodies.

TABLE 2
Antibody Binding Activity to Immobilized hFc-OX40L

| Sample | Binding Activity (EC₅₀ [nM]¹) |
|---|---|
| Ab A | 1 |
| Ab B | 0.6 |
| Ab C | 0.1 |
| Ab D | 0.2 |
| Ab E¹ | 0.69 |
| Ab G | 0.4 |
| Ab H | 0.73 |
| Ab I | 0.4 |
| hFc-OX40R | 1 |

¹EC₅₀ is the antibody concentration that is required, at a given ligand concentration, to obtain a binding signal that is 50% of the binding signal for antibody alone.
²Ab E has the same amino acid sequence as Ab F.

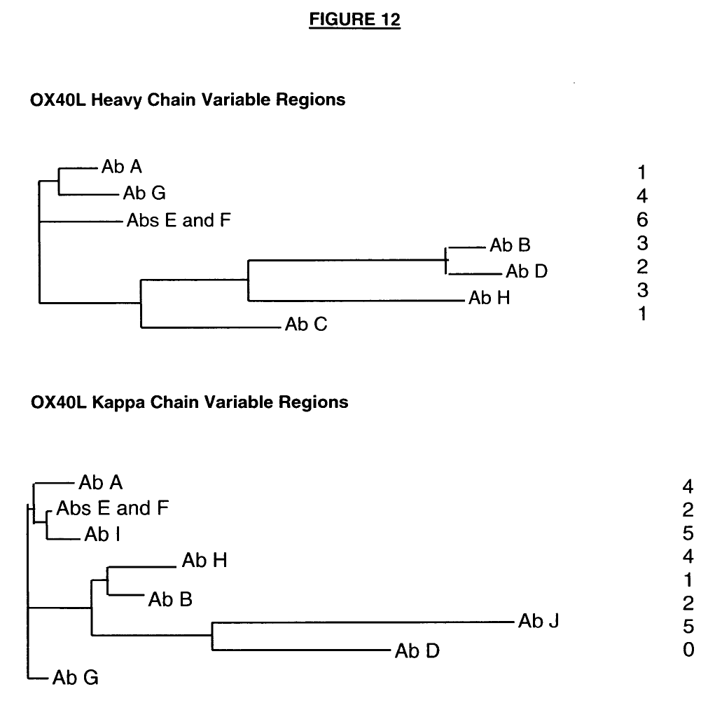

The amino acid sequences in the heavy chain variable regions of some of these antibodies are compared for sequence similarity. As shown in FIG. 12, these sequences fall into three major groups, with Ab A and Ab G in one group; Abs E and F in a second group; and Ab B, Ab D, Ab H, and Ab C in a third group. The amino acid sequences for Ab E and Ab F are identical. Likewise, the amino acid sequences of the light chain variable region in some of these antibodies are also compared. These sequences are also split into three groups with Ab A, Abs E and F, and Ab I in a first group; Ab H, Ab B, Ab J, and Ab D in a second group; and Ab G in a third group.

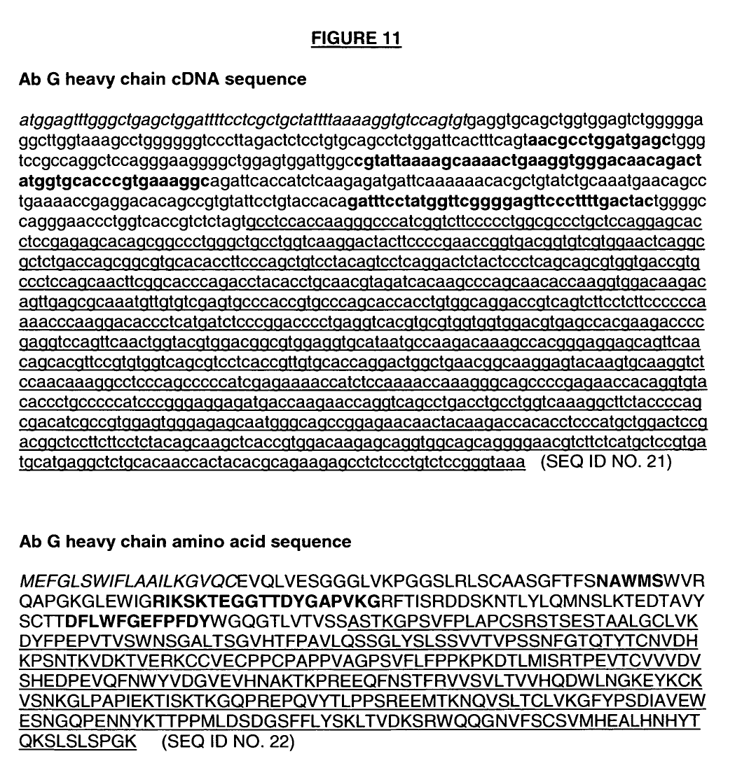

The cDNA nucleotide sequences and amino acid sequences of the heavy and light chains of Abs A-F are provided in FIGS. 1-10 and are identified as SEQ ID NOS. 1-20 as indicated in those figures. FIG. 11 provides the cDNA nucleotide sequence and amino acid sequence of the heavy chain in Ab G, which corresponds to SEQ ID NO. 21 and 22, respectively. As detailed in the examples below, certain of these monoclonal antibodies are tested in a variety of assays that address OX40L binding activity, the ability to block IL-2 production, and the ability to block OX40L stimulation of T cells.

### Example 2

### Relative Binding Affinity of Certain Anti-OX40L Human Monoclonal Antibodies to Human OX40L and Cynomolgus Monkey OX40L

The relative binding affinities of certain anti-OX40L MAbs were compared for binding to human OX40L and to cynomolgus monkey OX40L. Three individual beadsets were loaded by combining 270 μl of beads (Beadlyte Multi-Biotin Bead Kit (10 plex), 20× (2000 beads/μl) (Upstate Biotech. Cat# 41-012) with 20 ng of avidin-huIL-1 RFLAG (control), 20 ng avidin-hOX40L fusion, or 20 ng of avidin-cOX40L fusion in 15 ml centrifuge tubes (Corning cat #430052). Volumes were adjusted to 7.2 ml with PBST/1% BSA (PBS with 0.1% Tween-20/1% BSA) to normalize the protein concentration. Loading reactions were incubated at room temperature with mixing for at least 1 hour in the dark.

During this incubation, a 200 nm stock of each anti-OX40L antibody was prepared. Eight, 5-fold dilutions of each antibody stock were prepared in duplicate, resulting in antibody preparations ranging from 200 nm to 0.000512 nm. All dilutions were done in PBST/1% BSA.

Each bead loading reaction was transferred to a separate 50 ml filter top tube (0.45 μM; Corning cat# 430320), prewetting the membrane of the tube with PBST/1% BSA, and samples were gently aspirated. The beads were washed 3 times with 15 ml PBST (0.1% Tween-20 in PBS). After the last wash, each beadset was resuspended in 9 ml of PBST (180 wells×50 μl/well) by thoroughly washing the tube filters. Each beadset was separately mixed and 200 μl of each mixed beadsets (50 μl of each beadset/well×4 beadsets) were aliquotted to separate wells in 2 filter-bottom plates (Millipore cat # MABVN1210). A vacuum, using a Millipore vacuum system, was applied to the plate and the beads were then resuspended in 50 μl PBST/well.

Fifty μl of each 2× antibody dilution was added to the appropriate wells, so that each beadset was tested with each series of dilutions for each antibody. The resulting highest final concentration of anti-OX40L antibody was 100 nM, while the lowest final concentration was 0.000256 nm. The plates were incubated for 1.5 hours with mixing, protected from light. The Millipore vacuum system was used to wash beads 3 times with 250 μl PBST/well. To each well, 100 μl of 2 μg/ml anti-hulgG-PE (Rockland Immunochemicals) or anti-goat-PE (Rockland Immunochemicals cat# 705-708-125) diluted in PBST/1% BSA was added. Anti-goat-PE was used a negative control secondary antibody. The plates were incubated for 1 hour with mixing, protected from light, and were then washed 3 times using a Millipore vacuum system and 250 μl PBST/well. The beads were resuspended in 100 μl of PBST/well before analysis on a Luminex machine.

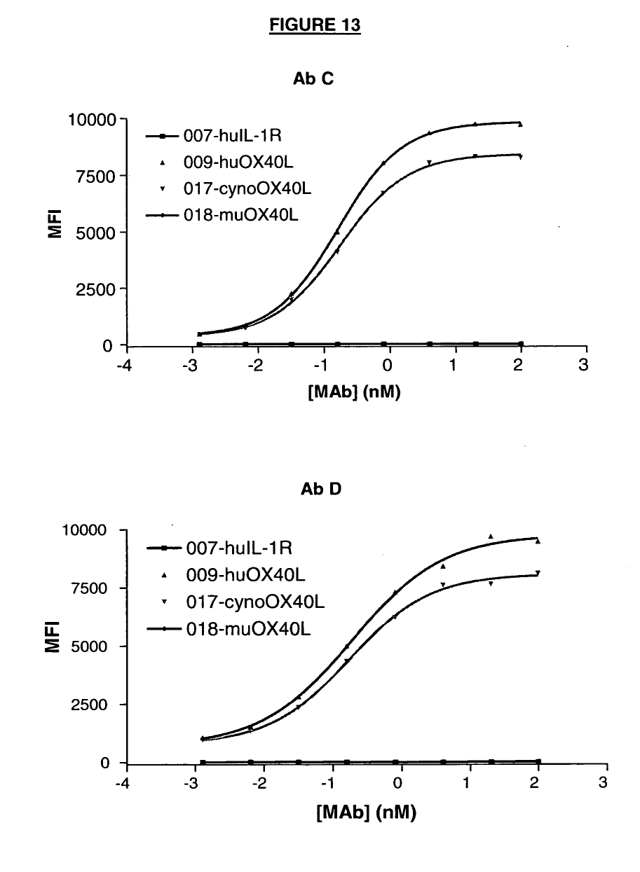

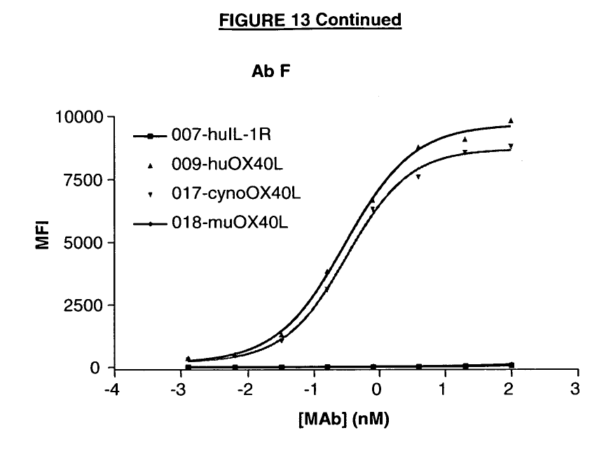

Samples were read by setting the Luminex machine to aspirate 75 μl of the 100 μl for each sample. The gates were set at 7109 and 18628. Binding to the human IL-1 receptor (IL-1R) attached to beads and to mouse OX40L attached to beads were included as negative control antigens for the assay. EC₅₀ values were then calculated from the resulting data. Table 2 lists the antibody EC₅₀ values. FIG. 13 provides raw binding data for three of the antibodies (Ab C, Ab D, and Ab F).

TABLE 3
Relative Binding Affinities

| | Relative Binding to | Relative Binding to |
|---|---|---|
| Antibody | Human OX40L¹ | Cynomolgus Monkey OX40L¹ |
| A | 0.180 | 0.302 |
| B | 0.780 | 0.769 |
| C | 0.162 | 0.176 |
| D | 0.211 | 0.173 |
| E | 0.564 | 0.606 |
| F | 0.299 | 0.321 |
| G | 0.360 | 0.340 |
| H | 0.316 | 0.399 |
| I | 0.524 | 0.496 |

¹Values are expressed as EC₅₀, which is the antibody concentration that is required, at a given ligand concentration, to obtain to obtain a binding signal that is 50% of the binding signal for antibody alone.

As set forth in Table 3 and FIG. 13, these antibodies bound to human OX40L (hOX40L) and cynomolgus monkey OX40L (cOX40L) at comparable levels.

### Example 3

### Binding Equilibrium of Certain Anti-OX40L MAbs and Competition for Binding Between Certain Anti-OX40L MAbs and OX40R

The binding equilibrium of four of the antibodies was assessed on a BIACore chip as described above in Example 1 with the following modifications. HFc-OX40L was immobilized to the sensor chip at a high density of 8,000 RU. Serial 2.5-fold dilutions of hFc-OX40L were prepared in sample buffer so that the final concentration of hFc-OX40L, once mixed with an anti-OX40L antibody, ranged between 20 nM to 0.005 nM. Monoclonal anti-OX40L antibody candidates were mixed with each hFc-OX40L dilution in a total of 400 μl so that the final concentration of monoclonal antibody was 0.2 nM. Samples were incubated at room temperature for at least five hours to allow samples to reach equilibrium. Samples were then injected over the immobilized hFc-OX40L surface for 30 minutes at 10 μl/min. After the sample injection, the samples were allowed to dissociate for 3 minutes, and then the surface was regenerated by injecting 8 mM glycine, pH 3.0, 1 M NaCl for 30 seconds. The binding signals obtained were proportional to the free antibody in solution at equilibrium for a given concentration of ligand. Plotting the binding signal versus ligand concentration, and using the scientific graphing software program GraphPad Prizm, the EC₅₀ values for each antibody at a given concentration in the presence of varying concentrations of hFc-OX40L were calculated.

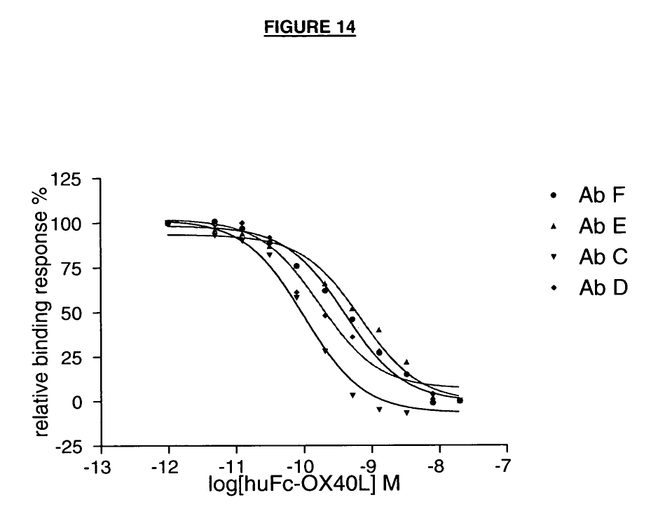

FIG. 14 provides a representative graph showing the binding signal data and Table 4 below provides the resulting EC₅₀ values.

TABLE 4
Equilibrium Binding Analysis

| Sample | EC₅₀¹ [M] |
|---|---|
| Ab F | 4.02 × 10⁻¹⁰ |
| Ab E | 6.864 × 10⁻¹⁰ |
| Ab C | 1.013 × 10⁻¹⁰ |
| Ab D | 1.701 × 10⁻¹⁰ |

¹The EC₅₀ is the antibody concentration that is required, at a given ligand concentration, to reduce the binding signal by 50% in comparison to the binding signal for ligand alone.

Some of the anti-OX40L antibodies were compared to OX40R for their binding affinities to OX40L immobilized on BIAcore chips or expressed on HUVEC cells. Specifically, BIACore chips were prepared as described above in Example 1, with the following modifications. HFc-OX40L was immobilized to the sensor chip at a high density of 8,000 RU. Monoclonal antibody candidates at two different final concentrations, 0.2 nM and 0.6 nM, or hOX40R at a final concentrations of 0.2 nM and 0.6 nM were incubated with varying final concentrations of 20 nM to 0.005 nM of hFc-OX40L, as described above. Samples were incubated at room temperature for at least five hours to allow samples to reach equilibrium. Samples were then injected over the immobilized hFc-OX40L surface for 30 minutes at 10 μl/min. After the sample injection, the samples were allowed to dissociate for 3 minutes, and then the surface was regenerated by injecting 8 mM glycine, pH 3.0, 1 M NaCl for 30 seconds. The binding signals, measured in RU, obtained were proportional to the free antibody in solution at equilibrium for a given concentration of ligand. The dissociation equilibrium constant (K_{D}) was obtained from nonlinear regression analysis of the competition curves using a dual-curve one-site homogeneous binding model (KinExA software v. 2.3, Sapidyne Instruments Inc., Boise Id.).

TABLE 5
Binding Affinities of anti-OX40L MAbs Compared to OX40R

| Sample | Affinity (K_{D}) |
|---|---|
| Ab F | 120 pM |
| Ab D | 390 pM |
| Ab C | 33 pM |
| Fc-OX40R | 1000 pM |

As shown in Table 5, the anti-OX40L antibodies had superior binding affinities compared to the OX40R.

Studies were performed with human embryonic vein endothelial cells (HUVECs; Clonetics CC-2571, lot # OF0611). HUVEC cells, which naturally express OX40L, were grown to confluency and passed 4 to 6 times before use. Cells were removed from the tissue culture flask with trypsin and washed 2× with PBS by centrifuging the cells at 400-500×g and discarding the media first and PBS second. Samples were prepared by suspending 300,000 cells in 100 μl of FACS buffer (0.1% BSA, 0.01% sodium azide in PBS). Cells were then pre-treated with 20 μg/ml (final concentration) human Ig for 5 minutes at room temperature. Next, the anti-OX40L antibody test reagent, at a final concentration of 15, 3, 0.6, 0.12, 0.024, or 0.0048 μg/ml, was added to cells. Cells were incubated with these antibodies for about 10 minutes on ice. Then, biotintylated hFc-OX40R at a final concentration of 3 μg/ml was added to all samples. HFc-OX40R alone was used as a positive control, while hFc alone, HUVEC alone, and PE-SA (PE-streptavidin) alone were used as negative controls. These samples were allowed to incubate for 20 additional minutes, cells were washed, and resuspended in FACS buffer containing (1:100) PE-SA for 20 minutes on ice. Cells were washed again in ice cold PBS and resuspended in 0.5 ml in 1% formaldehyde in ice cold PBS and immediately read by flow cytometer.

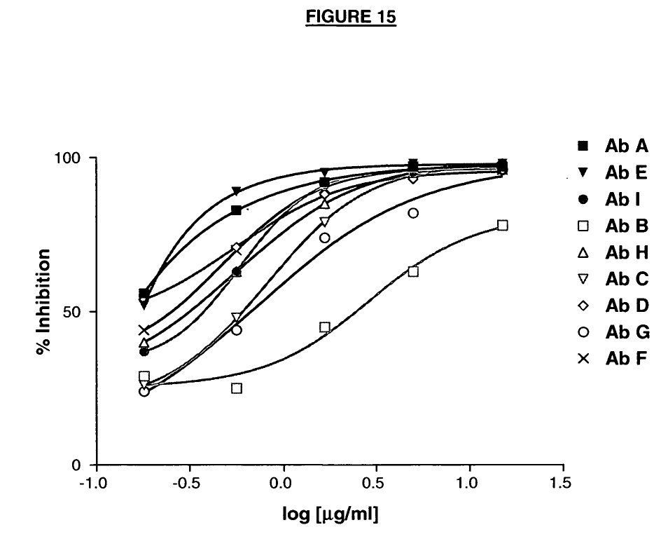

FIG. 15 shows the results from each of the tested anti-OX40L antibodies compared to the hFc-OX40R protein.

### Example 4

### Evaluating Inhibition of IL-2 Production by Human T Cells

Certain anti-OX40L MAbs were assessed for their ability to block the production of IL-2 by human T cells using a whole blood assay. Specifically, a human whole blood assay was developed based on the knowledge that OX40L co-stimulation leads to an increase in IL-2 production by T cells. Human whole blood was diluted 50% by adding an equal volume of Iscoves media (Gibco). Plates (96 wells; Falcon Inc.) were coated with a solution of 10 μg/ml of anti-CD3 (R&D system), diluted in PBS, by adding 100 μl of the anti-CD3 solution to each well and incubating at 4° C. overnight. The coated plates were washed using 200 μl of PBS. Diluted whole blood was added to each well and hFc-OX40L (soluble), diluted in Iscoves media (Gibco), was added to a final concentration of 1.5 nM. The blood was cultured for 48 hours at 37° C. and cells were pelleted by centrifuging at 400×g. The supernatant was removed and assayed by ELISA for IL-2 protein using a R&D System IL-2 ELISA kit according to manufacturer's instructions.

Antibodies were tested by adding increasing concentrations of antibody to the co-stimulation reactions and the effect on IL-2 production was determined (IC₅₀s). IC₅₀ were calculated as the concentration of antibody that reduces the amount of IL-2 by 50%. It was determined that the level of hFc-OX40L used gave a strong, reproducible, signal-noise ratio. However, because of the amount of hFc-OX40L used, the assay is limited in its ability to differentiate the potency of antibodies with sub nM potency because of the need for stoichiometric amounts of the antibody to neutralize this amount of ligand. Less potent antibodies were readily differentiated by the assay. Fc-OX40R was used as a positive control while human IgG was used as a negative control for the assay.

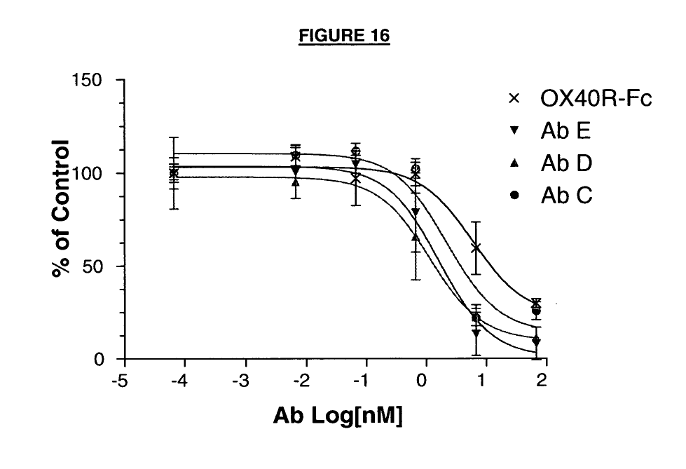

As shown in Table 6, the anti-OX40L antibodies inhibited IL-2 production in whole blood better than the OX40R. FIG. 16 provides a representative graph of data from an IL-2 production inhibition assay.

TABLE 6
Inhibition of IL-2 Production

| Sample | IL-2 Inhibition (IC₅₀ [nM]) |
|---|---|
| Ab A | 0.32 |
| Ab B | 0.49 |
| Ab C | 2 |
| Ab D | ND¹ |
| Ab E | 0.58 |
| Ab G | 0.22 |
| Ab H | 1.6 |
| Ab I | 0.26 |
| Fc-OX40R | 5.2 |

¹Not done.

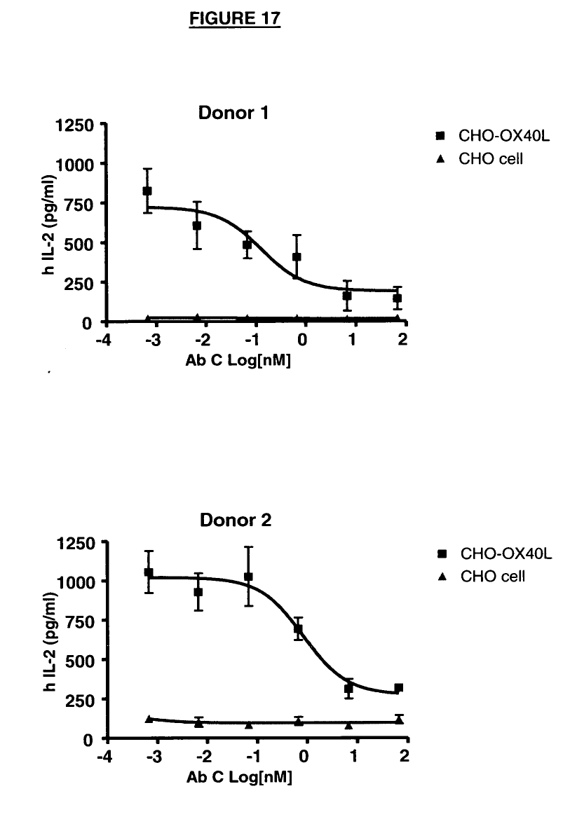

A similar assay was performed to measure the ability of AbC to block IL-2 production. This whole blood assay was performed as described above except CHO cells expressing hOX40L were used instead of soluble hFc-OX40L. The parental CHO cell line was used as a negative control. The results of this modified assay are shown in FIG. 17.

### Example 5

### Evaluating Inhibition of IL-2 Production by Cynomolgus Monkey T Cells

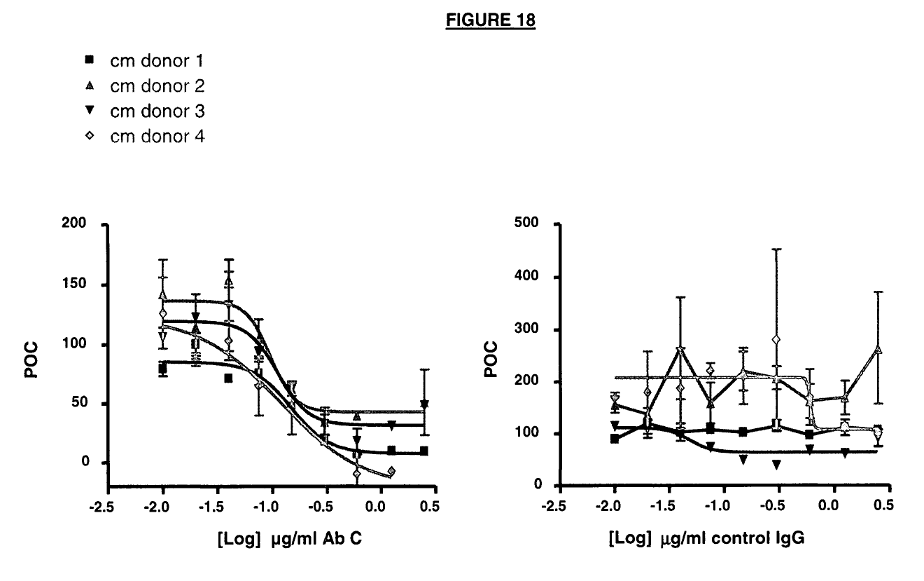

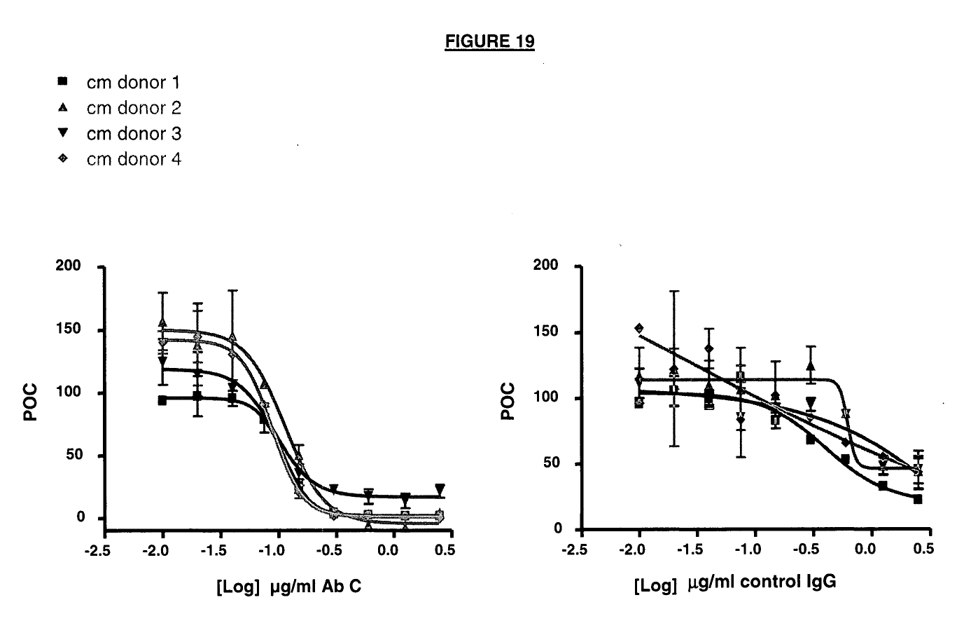

Ab C was assessed for its ability to block the production of IL-2 by cynomolgus monkey T cells. Plates (96 wells; Falcon Inc.) were coated with a solution of 1 μg/ml of anti-CD3 (R&D system), diluted in PBS, by adding 100 μl of the anti-CD3 solution to each well and incubating at 4° C. overnight. The coated plates were washed using 200 μl of PBS. The plates were then coated with a solution of either 2.5 μg/ml (FIG. 18) or 1.25 μg/ml (FIG. 19) of hFc-OX40L (soluble), diluted in PBS, by adding 100 μl of the hFc-OX40L solution to each well and incubating at 37° C. for 4 hours. The plates were washed again using 200 μl of PBS before T cells were added.

T cells from 4 cynomolgus monkey blood donors were purified by using the a Miltynl Biotec kit (catalog # 130-091-156) for purifying human T cells using negative selection by following the manufacturer's instructions with the following exception. After incubating the samples with the biotintylated antibodies provided in the kit, streptavidin-coated magnetic beads (to bind to the biotintylated antibodies) and anti-monkey CD20 magnetic beads (Miltynl Biotec catalog # 130-091-105) were added to the samples and incubated according to the T cell kit's instructions before loading the samples onto a magnetic column for final T cell purification. The anti-monkey CD20 magnetic beads were used to completely remove B cells.

Cynomolgus monkey T cells were resuspended in assay media (RPMI 1640, 10% FBS, PSG (penicillin, streptomycin, and glutinin), NEAA (non-essential amino acids), and β-mercaptoethanol) and 100,000 T cells in 100 μl were added to each well. Varying concentrations of Ab C or control IgG were tested by adding 100 μl of antibody solutions per well to attain final concentrations ranging from 2.5 μg/ml to 0.01 μg/ml. The T cells were cultured for 48 hours at 37° C., 5% CO₂ and cells were pelleted by centrifuging at 400×g. A 100 μl volume of the supernatant was removed and assayed by ELISA for IL-2 protein using a BD Pharmingen IL-2 ELISA kit (catalog #551494) according to manufacturer's instructions. ELISA OD values were converted to POC (percent of control) values for graphic analysis.

As shown in FIGS. 18 and 19, Ab C inhibited IL-2 production by co-stimulated cynomolgus monkey T cells.

### Example 6

### Evaluating Inhibition of OX40L Mediated T Cell Proliferation

Certain anti-OX40L antibodies were tested for their ability to block T cell costimulation mediated by OX40L and CD3. Round bottom 96-well plates were coated with anti-CD3 (Pharmingen #555336) overnight at 4° C. Because T cells were freshly harvested from individual donors, each T cell preparation required an empirical determination of the optimal concentration of anti-CD3 necessary to result in optimal stimulation. Thus, solutions of anti-CD3 ranging from 0.25 μg/ml to 4.0 μg/ml were used to determine the appropriate concentration to use with a particular T cell preparation. The plates were washed with 200 μl of PBS. The anti-CD3 coated plates were then coated with an 11 nm solution of hFc-OX40L for 4 hours at 37° C. Plates were then washed with 200 μl of PBS, as described above.

Peripheral blood mononuclear cells (PBMCs) were isolated from Leukopheresis packs using Ficoll-Paque density gradients (Pharmacia). T cells were isolated from the PBMCs using Pan T cell isolation kits from Miltenyi Biotec (cat# 130-053-001), using the manufacturer's instructions. Isolated T cells were diluted to 1×10⁶/ml in RPMI plus 10% fetal calf serum (FCS) and 100 μl of these diluted cells were added to the anti-CD3/hFc-OX40L coated plates. The anti-OX40L antibodies that were tested were individually diluted to 6 μg/ml and then further diluted in serial three fold dilutions that spanned final concentrations of 19 nM to 0.078 nM. 100 μl of each antibody dilution was added to the 100 μl of T cells in separate wells. Human IgG replaced the anti-OX40L antibodies as a negative control for this assay (i.e., no blocking). OX40R-Fc was used in place of the antibodies for a positive control (i.e., with blocking). Plates were incubated for 48 hours at 37° C., 5% CO₂. 1 μCi/well of ³H-thymidine (ICN, cat # 2404205) was then added. Plates were incubated for 16 hours 37° C., 5% CO₂. Cells were harvested using a Tomtec harvester. ³H-thymidine uptake was measured using a Microbeta Trilux Liquid Scintillation counter (Perkin Elmer).

As discussed above, for IC₅₀ determinations, antibodies were tested from 19 nM to 0.078 nM, in 3-fold dilutions and in triplicates. Depending on the T cell donor, various amounts of anti-CD3, and 11 nM of hFc-OX40L was used to stimulate the T cells. Average ³H-thymidine incorporation values (of triplicates) were expressed as percent of control. Inhibition curves were plotted (3H-thymidine incorporation (POC) vs. log antibody concentration) and IC₅₀ values determined by nonlinear regression (sigmoidal dose response with variable slope) using GraphPad (PRISM™) software. All results were expressed as the mean±standard error bar mean (SEM).

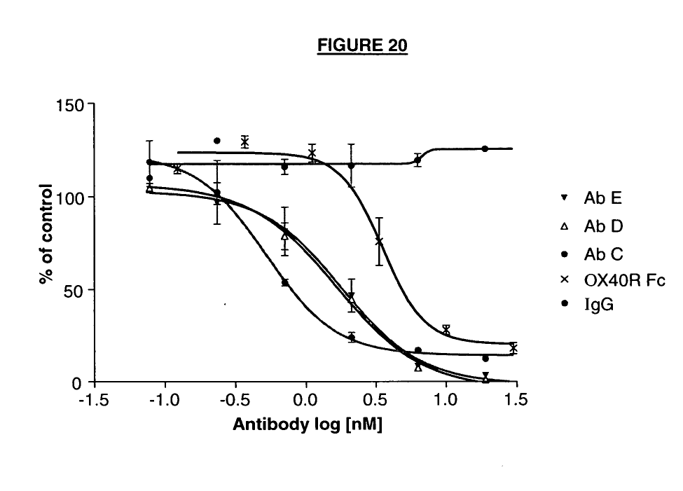

Table 7 and FIG. 20, show the results of the T cell proliferation inhibition assay.

TABLE 7
Inhibition of T cell Proliferation

| Sample | Costimulation Value (IC₅₀)¹ |
|---|---|
| Ab A | 1.2 |
| Ab B | 2.1 |
| Ab C | 0.5 |
| Ab D | 1.4 |
| Ab E | 1.8 |
| Ab G | 3.2 |
| Ab H | 2.2 |
| Ab I | 2.4 |
| Fc-OX40R | 3.6 |

¹The IC₅₀ is the antibody concentration that is required, at a given ligand concentration, to reduce the proliferation signal by 50% in comparison to the proliferation signal for ligand alone.

### Example 7

### Evaluating Binding of Certain Anti-OX40L Antibodies to CHO Cell OX40L and Evaluating Neutralization of Certain OX40R Binding to CHO Cell OX40L

Chinese hamster ovary (CHO) (i.e., ATCC CCL-61) cells are transfected to allow cell surface expression of OX40L. These cells are prepared by stably transfecting CHO cells with a Fc-cOX40L plasmid, linearized with Pvul. CHO cells are plated at 1.5×10⁶ so that the cells are 80-90% confluent when performing the transfection. Transfection reagent FuGene™ 6 (Roche, Cat. No. 1 814 443) is used for stable transfection. Twenty-four μl of FuGene™ 6 is diluted into 800 μl of MEM serum free medium and 8 μg of the linerized plasmid is added followed by an incubation at room temperature for 20 minutes. FuGene™ 6/DNA mix is added to CHO cells in a 100 mm plate followed by an incubation for 48 hours in 5% CO₂, 37° C. incubator. CHO cells are grown in DMEM high glucose (Gibco); 5% FBS, 1× pen/strep; glutamine, 1× Non essential aa; 1× Na Pyruvate; and 1×HT supplement.

After the 48 hour incubation, the cells are split 1:10 into HT minus selection media (DMEM high glucose (Gibco); 5% dialyzed FBS; 1× pen/strep, glutamine; 1× Non essential aa; 1× Na Pyruvate). Then cells are grown at 5% CO₂, 37° C. incubator, changing the selection medium twice a week. Colonies appear after two weeks of selection and are isolated into 6 well plates by cloning disc and grown in 5% CO₂ at 37° C. When cells are confluent in 6 well plates, huOX40L expression is detected by FACS with hFc-OX40R.

COX40L expressing CHO cells are used to compare anti-OX40L antibodies to cFc-OX40R (human Fc region and cynomolgus monkey OX40R) for binding to membrane associated cOX40L. Specifically, transfected CHO cells are grown to confluence in RPMI media and are harvested using Versene. Cells are washed in FACS buffer (2% fetal bovine serum (heat inactivated), 0.1% sodium azide in PBS buffer) with spinning at 400×g. CHO cells are then resuspended in FACS buffer and so that 5×10⁵ cells are introduced into each sample tube.

The anti-OX40L antibodies being tested, cFc-OX40R, and human IgG (negative control) staining reagents are separately diluted in ice cold FACS buffer to give final staining concentrations of 45, 15, 5, 1.7, 0.6, and 0.2 μg/ml for each staining reagent. Cells are stained with one of anti-OX40L antibodies, cFc-OX40R, or hIgG in 100 μl of staining reagent. Cells are then incubated on ice for 1 hour followed by 3 washes in FACS buffer. Goat anti human IgG Fc-FITC is diluted 1:1000 in cold FACS buffer and 100 μl is added to the washed cells in each sample. Cells are incubated on ice for 30 minutes and then washed 3 times. After the final wash, stained cells are resuspended in 500 μl cold FACS buffer and are kept on ice until analysis on a FACSCalibur (Becton Dickinson).

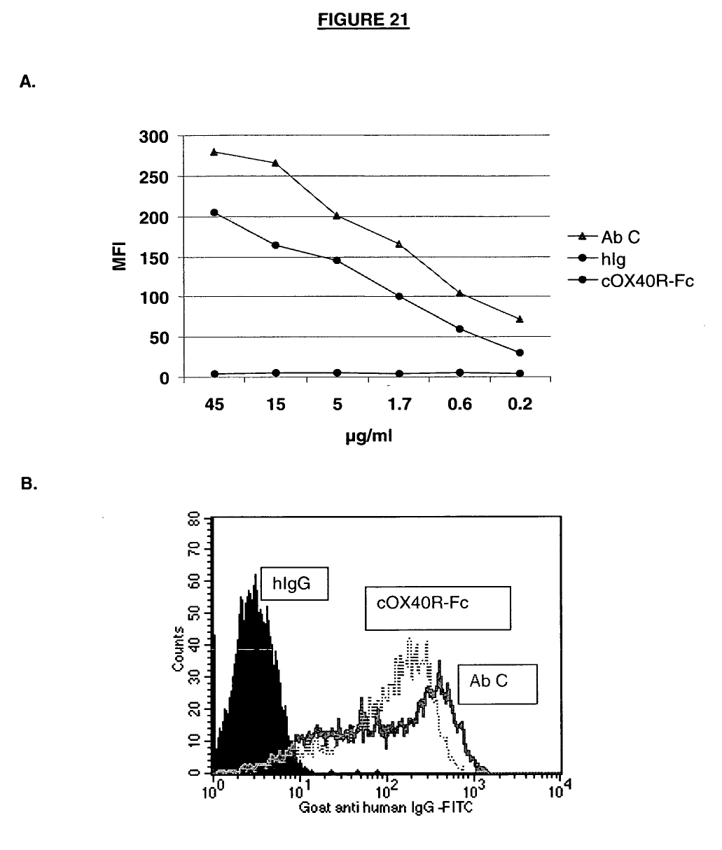

FIG. 21A provides the results of the FACS analysis.

Ab C, a representative antibody from the identified group of anti-OX40L antibodies, is also tested for its ability to neutralize binding of cOX40R-Fc to OX40L expressed on CHO cells. Transfected CHO cells are prepared as described above and incubated with 100 μl of AbC or hIgG at the final staining concentrations listed above and under the conditions described above. After washing the cells three times, they are then incubated with 100 μl of biotintylated (biotintylation kit from Pierce) cFc-OX40R (at 5 μg/ml diluted in cold FACS buffer) on ice for 1 hour. Cells are washed three times as described above. Streptavidin-PE is diluted 1:500 in cold (4° C.) FACS buffer and 100 μl is added to the washed cells, which are then incubated for 30 minutes on ice. Cells are washed 3 times and resuspended in 500 μl of cold FACS buffer for analysis as described above.

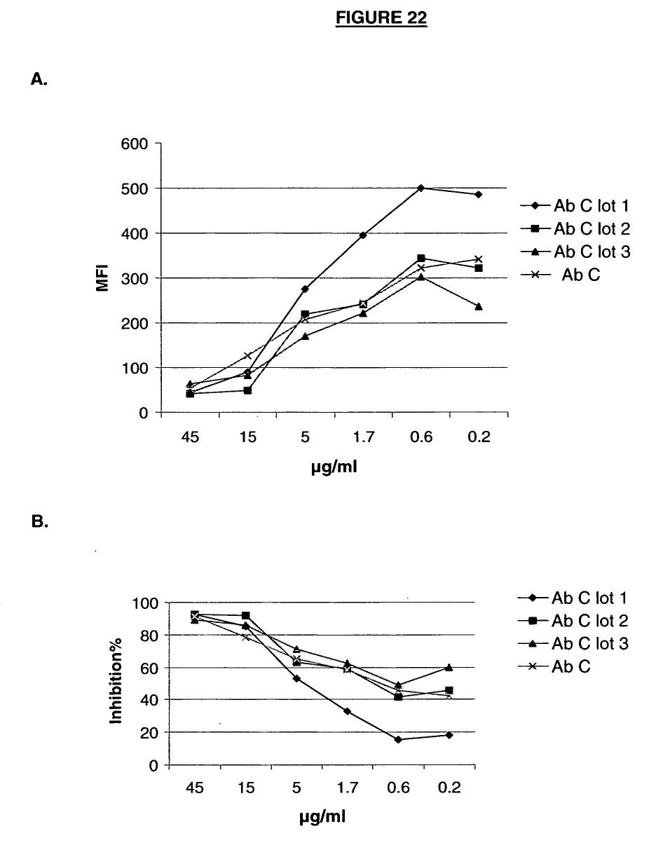

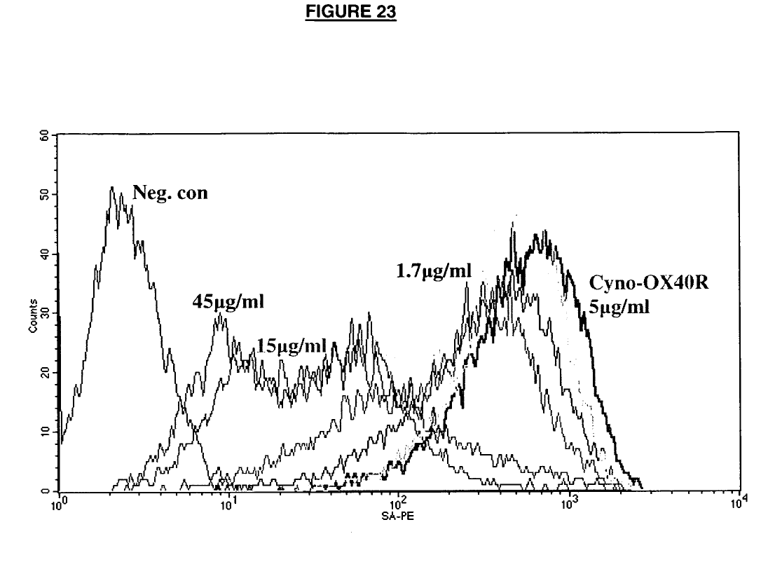

As shown in FIG. 22, Ab C reduces the ability of cFc-OX40R to bind to membrane associated OX40L on CHO cells. In addition, Ab C obtained from different sources shows minimal variation in activity, indicating that Ab C can be produced via several expression systems. FIG. 23 provides an exemplary FACS analysis comparing the activity of Ab C at various concentrations.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. An isolated polypeptide comprising at least one complementarity determining region (CDR) selected from CDR1a, CDR2a, or CDR3a
wherein CDR1a comprises the amino acid sequence a b c d e, wherein amino acid a is selected from asparagine, threonine, phenylalanine, or serine; amino acid b is selected from alanine or tyrosine; amino acid c is selected from tryptophan, tyrosine, or glycine; amino acid d is selected from methionine or tryptophan; and amino acid e is selected from serine, asparagine, or histidine;
wherein CDR2a comprises the amino acid sequence f g h i j k l m n o p q r s t, wherein amino acid f is selected from arginine or valine; amino acid g is isoleucine; amino acid h is selected from lysine, tyrosine, or tryptophan; amino acid i is selected from serine, isoleucine, tyrosine, threonine, or arginine; amino acid j is selected from lysine, serine, or aspartic acid; amino acid k is selected from threonine or glycine; amino acid l is selected from aspartic acid, serine, or glutamic acid; amino acid m is selected from glycine, threonine, or asparagine; amino acid n is selected from glycine, asparagine, lysine, or threonine; amino acid o is selected from threonine or tyrosine; amino acid p is selected from threonine, isoleucine, asparagine, or tyrosine; amino acid q is selected from aspartic acid, proline, or alanine; amino acid r is selected from tyrosine, serine, or aspartic acid; amino acid s is selected from glycine, alanine, leucine, or serine; and amino acid t is selected from alanine, lysine, or valine;
wherein CDR3a comprises the amino acid sequence u v w x y z a′ b′ c′ d′ e′ f′, wherein amino acid u is selected from aspartic acid, glycine, methionine, or serine; amino acid v is selected from arginine, glycine, aspartic acid, tyrosine, or phenylalanine; amino acid w is selected from tyrosine, valine, glycine, or leucine; amino acid x is selected from phenylalanine, aspartic acid, tyrosine, or tryptophan; amino acid y is selected from phenylalanine, aspartic acid, tyrosine, or isoleucine; amino acid z is selected from glycine, tyrosine, proline, valine, or phenylalanine; amino acid a′ is selected from glutamic acid, serine, tyrosine, tryptophan, or alanine; amino acid b′ is selected from phenylalanine, glycine, tyrosine, threonine, or serine; amino acid c′ is selected from proline, tyrosine, serine, lysine, or glycine; amino acid d′ is selected from phenylalanine, tyrosine, or glycine; amino acid e′ is selected from aspartic acid, tyrosine, arginine, or histidine; and amino acid f′ is selected from tyrosine, valine, glycine, arginine, or threonine; and
wherein the polypeptide, in association with an antibody light chain, is capable of binding OX40L.

2. The isolated polypeptide of claim 1, wherein the polypeptide comprises an antibody heavy chain variable region.

3. The isolated polypeptide of claim 2, further comprising an antibody heavy chain constant region.

4. The isolated polypeptide of claim 3, wherein the antibody heavy chain variable region and the heavy chain constant region comprise an amino acid sequence as set forth in SEQ ID NO. 2; SEQ ID NO. 6; SEQ ID NO. 10; SEQ ID NO. 14; SEQ ID NO. 18; or SEQ ID NO. 22.

5. The isolated polypeptide of claim 1, comprising CDR2a wherein CDR2a comprises the amino acid sequence f g h i j k l m n o p q r s t g′ and wherein amino acid g′ is selected from proline, lysine, or serine.

6. The isolated polypeptide of claim 5, comprising CDR2a wherein CDR2a comprises the amino acid sequence f g h i j k l m n o p q r s t g′ h′ and wherein amino acid h′ is selected from valine or glycine.

7. The isolated polypeptide of claim 6, comprising CDR2a wherein CDR2a comprises the amino acid sequence f g h i j k l m n o p q r s t g′ h′ i′ and wherein amino acid i′ is lysine.

8. The isolated polypeptide of claim 7, comprising CDR2a wherein CDR2a comprises the amino acid sequence f g h i j k l m n o p q r s t g′ h′ i′ j′ and wherein amino acid j′ is glycine.

9. The isolated polypeptide of claim 1, comprising CDR3a wherein CDR3a comprises the amino acid sequence u v w x y z a′ b′ c′ d′ e′ f′ k′ and wherein amino acid k′ is selected from aspartic acid, methionine, asparagine, tyrosine, or valine.

10. The isolated polypeptide of claim 9, comprising CDR3a wherein CDR3a comprises the amino acid sequence u v w x y z a′ b′ c′ d′ e′ f′ k′ l′ and wherein amino acid l′ is selected from histidine, aspartic acid, serine, tyrosine, or phenylalanine.

11. The isolated polypeptide of claim 10, comprising CDR3a wherein CDR3a comprises the amino acid sequence u v w x y z a′ b′ c′ d′ e′ f′ k′ l′ m′ and wherein amino acid m′ is selected from valine, aspartic acid, or glycine.

12. The isolated polypeptide of claim 11, comprising CDR3a wherein CDR3a comprises the amino acid sequence u v w x y z a′ b′ c′ d′ e′ f′ k′ l′ m′ n′ and wherein amino acid n′ is selected from phenylalanine, methionine, or tyrosine.

13. The isolated polypeptide of claim 12, comprising CDR3a wherein CDR3a comprises the amino acid sequence u v w x y z a′ b′ c′ d′ e′ f′ k′ l′ m′ n′ o′ and wherein amino acid o′ is aspartic acid.

14. The isolated polypeptide of claim 13, comprising CDR3a wherein CDR3a comprises the amino acid sequence u v w x y z a′ b′ c′ d′ e′ f′ k′ l′ m′ n′ o′ p′ and wherein amino acid p′ is selected from valine or tyrosine.

15. The isolated polypeptide of claim 1, comprising at least two complementarity determining regions (CDRs) selected from the CDR1a, CDR2a, or CDR3a of claim 1, wherein the polypeptide, in association with an antibody light chain, is capable of binding OX40L.

16. The isolated polypeptide of claim 1, comprising the CDR1a, CDR2a, and CDR3a of claim 1, wherein the polypeptide, in association with an antibody light chain, is capable of binding OX40L.

17. An antibody fragment selected from a Fab, a Fab′, a F(ab′)2, a Fv, a Facb, and a single-chain antibody, wherein the antibody fragment comprises the isolated polypeptide of claim 1.

18. An isolated polypeptide comprising at least one complementarity determining region (CDR) selected from at least one of
amino acids 50 to 54 of SEQ ID NO. 2;
amino acids 69 to 87 of SEQ ID NO. 2;
amino acids 120 to 135 of SEQ ID NO. 2;
amino acids 50 to 54 of SEQ ID NO. 6;
amino acids 69 to 84 of SEQ ID NO. 6;
amino acids 117 to 134 of SEQ ID NO. 6;
amino acids 50 to 54 of SEQ ID NO. 10;
amino acids 69 to 85 of SEQ ID NO. 10;
amino acids 118 to 135 of SEQ ID NO. 10;
amino acids 50 to 54 of SEQ ID NO. 14;
amino acids 69 to 84 of SEQ ID NO. 14;
amino acids 117 to 131 of SEQ ID NO. 14;
amino acids 50 to 54 of SEQ ID NO. 18;
amino acids 69 to 87 of SEQ ID NO. 18;
amino acids 120 to 133 of SEQ ID NO. 18;
amino acids 50 to 54 of SEQ ID NO. 22;
amino acids 69 to 87 of SEQ ID NO. 22; or
amino acids 120 to 131 of SEQ ID NO. 22
wherein the polypeptide, in association with an antibody light chain, is capable of binding OX40L.

19. An isolated polypeptide comprising at least two of the complementarity determining regions (CDRs) of claim 18, wherein the polypeptide, in association with an antibody light chain, is capable of binding OX40L.

20. An isolated polypeptide comprising at least three of the complementarity determining regions (CDRs) of claim 18, wherein the polypeptide, in association with an antibody light chain, is capable of binding OX40L.

21. The isolated polypeptide of claim 18, wherein the isolated polypeptide comprises amino acids 50 to 54 of SEQ ID NO. 2, amino acids 69 to 87 of SEQ ID NO. 2, and amino acids 120 to 135 of SEQ ID NO. 2.

22. The isolated polypeptide of claim 18, wherein the isolated polypeptide comprises amino acids 50 to 54 of SEQ ID NO. 6, amino acids 69 to 84 of SEQ ID NO. 6, and amino acids 117 to 134 of SEQ ID NO. 6.

23. The isolated polypeptide of claim 18, wherein the isolated polypeptide comprises amino acids 50 to 54 of SEQ ID NO. 10, amino acids 69 to 85 of SEQ ID NO. 10, and amino acids 118 to 135 of SEQ ID NO. 10.

24. The isolated polypeptide of claim 18, wherein the isolated polypeptide comprises amino acids 50 to 54 of SEQ ID NO. 14, amino acids 69 to 84 of SEQ ID NO. 14, and amino acids 117 to 131 of SEQ ID NO. 14.

25. The isolated polypeptide of claim 18, wherein the isolated polypeptide comprises amino acids 50 to 54 of SEQ ID NO. 18, amino acids 69 to 87 of SEQ ID NO. 18, and amino acids 120 to 133 of SEQ ID NO. 18.

26. The isolated polypeptide of claim 18, wherein the isolated polypeptide comprises amino acids 50 to 54 of SEQ ID NO. 22, amino acids 69 to 87 of SEQ ID NO. 22, and amino acids 120 to 131 of SEQ ID NO. 22.

27. An isolated polypeptide comprising at least one complementarity determining region (CDR) selected from CDR1b, CDR2b, or CDR3b
wherein CDR1b comprises the amino acid sequence a1 b1 c1 d1 e1 f1 g1 h1 i1 j1 k1, wherein amino acid a1 is arginine; amino acid b1 is selected from alanine or serine; amino acid c1 is serine; amino acid d1 is glutamine; amino acid e1 is selected from glycine or serine; amino acid f1 is selected from isoleucine, valine, or leucine; amino acid g1 is selected from serine or valine; amino acid h1 is selected from asparagine, serine, or histidine; amino acid i1 is selected from histidine, asparagine, serine, or tyrosine; amino acid j1 is selected from leucine, tyrosine, or aspartic acid; and amino acid k1 is selected from valine, leucine, glycine, or asparagine;
wherein CDR2b comprises the amino acid sequence l1 m1 n1 o1 p1 q1 r1, wherein amino acid l1 is selected from alanine, glycine, or lysine; amino acid m1 is selected from alanine or lysine; amino acid n1 is selected from serine or phenylalanine; amino acid o1 is selected from threonine, serine, or asparagine; amino acid p1 is selected from leucine or arginine; amino acid q1 is selected from glutamine, alanine, or phenylalanine; and amino acid r1 is selected from serine or threonine;
wherein CDR3b comprises the amino acid sequence s1 t1 u1 v1 w1 x1 y1 z1 a1′, wherein amino acid s1 is selected from glutamine or methionine; and amino acid t1 is selected from lysine or glutamine; amino acid u1 is selected from tyrosine, alanine, serine, or phenylalanine; amino acid v1 is selected from asparagine, glycine, threonine, or tyrosine; amino acid w1 is selected from serine, glycine, or glutamine; amino acid x1 is selected from alanine, serine, isoleucine, or threonine; amino acid y1 is selected from proline or leucine; amino acid z1 is selected from leucine, tryptophan, or phenylalanine; and amino acid a1′ is threonine; and
wherein the polypeptide, in association with an antibody heavy chain, is capable of binding OX40L.

28. The isolated polypeptide of claim 27, wherein the polypeptide comprises an antibody light chain variable region.

29. The isolated polypeptide of claim 28, further comprising an antibody light chain constant region.

30. The isolated polypeptide of claim 29, wherein the antibody light chain variable region and the light chain constant region comprise an amino acid sequence as set forth in SEQ ID NO. 4; SEQ ID NO. 8; SEQ ID NO. 12; SEQ ID NO. 16; or SEQ ID NO. 20.

31. The isolated polypeptide of claim 27, comprising CDR1b wherein CDR1b comprises the amino acid sequence a1 b1 c1 d1 e1 f1 g1 h1 i1 j1 k1 b1′ and wherein amino acid b1′ is selected from asparagine or alanine.

32. The isolated polypeptide of claim 31, comprising CDR1b wherein CDR1b comprises the amino acid sequence a1 b1 c1 d1 e1 f1 g1 h1 i1 j1 k1 b1′ c1′ (SEQ ID NO. 23) and wherein amino acid c1′ is threonine.

33. The isolated polypeptide of claim 32, comprising CDR1b wherein CDR1b comprises the amino acid sequence a1 b1 c1 d1 e1 f1 g1 h1 i1 j1 k1 b1′ c1′ d1′ (SEQ ID NO. 24) and wherein amino acid d1′ is tyrosine.

34. The isolated polypeptide of claim 33, comprising CDR1b wherein CDR1b comprises the amino acid sequence a1 b1 c1 d1 e1 f1 g1 h1 i1 j1 k1 b1′ c1′ d1′ e1′ (SEQ ID NO. 25) and wherein amino acid e1′ is leucine.

35. The isolated polypeptide of claim 34, comprising CDR1b wherein CDR1b comprises the amino acid sequence a1 b1 c1 d1 e1 f1 g1 h1 i1 j1 k1 b1′ c1′ d1′ e1′ f′ (SEQ ID NO. 26) and wherein amino acid f1′ is serine.

36. The isolated polypeptide of claim 27, comprising at least two complementarity determining regions (CDRs) selected from the CDR1b, CDR2b, or CDR3b of claim 27, wherein the polypeptide, in association with an antibody heavy chain, is capable of binding OX40L.

37. The isolated polypeptide of claim 27, comprising the CDR1b, CDR2b, and CDR3b of claim 27, wherein the polypeptide, in association with an antibody heavy chain, is capable of binding OX40L.

38. An antibody fragment selected from a Fab, a Fab′, a F(ab′)2, a Fv, a Facb, and a single-chain antibody, wherein the antibody fragment comprises the isolated polypeptide of claim 27.

39. An isolated polypeptide comprising at least one complementarity determining region (CDR) selected from at least one of
amino acids 46 to 56 of SEQ ID NO. 4;
amino acids 72 to 78 of SEQ ID NO. 4;
amino acids 111 to 119 of SEQ ID NO. 4;
amino acids 46 to 56 of SEQ ID NO. 8;
amino acids 72 to 78 of SEQ ID NO. 8;
amino acids 111 to 119 of SEQ ID NO. 8;
amino acids 44 to 59 of SEQ ID NO. 12;
amino acids 75 to 81 of SEQ ID NO. 12;
amino acids 114 to 122 of SEQ ID NO. 12;
amino acids 44 to 55 of SEQ ID NO. 16;
amino acids 71 to 77 of SEQ ID NO. 16;
amino acids 110 to 118 of SEQ ID NO. 16;
amino acids 46 to 56 of SEQ ID NO. 20;
amino acids 72 to 78 of SEQ ID NO. 20; or
amino acids 111 to 119 of SEQ ID NO. 20
wherein the polypeptide, in association with an antibody heavy chain, is capable of binding OX40L.

40. An isolated polypeptide comprising at least two of the complementarity determining regions (CDRs) of claim 39, wherein the polypeptide, in association with an antibody heavy chain, is capable of binding OX40L.

41. An isolated polypeptide comprising at least three of the complementarity determining regions (CDRs) of claim 39, wherein the polypeptide, in association with an antibody heavy chain, is capable of binding OX40L.

42. The isolated polypeptide of claim 39, wherein the isolated polypeptide comprises amino acids 46 to 56 of SEQ ID NO. 4, amino acids 72 to 78 of SEQ ID NO. 4, and amino acids 111 to 119 of SEQ ID NO. 4.

43. The isolated polypeptide of claim 39, wherein the isolated polypeptide comprises amino acids 46 to 56 of SEQ ID NO. 8, amino acids 72 to 78 of SEQ ID NO. 8, and amino acids 111 to 119 of SEQ ID NO. 8.

44. The isolated polypeptide of claim 39, wherein the isolated polypeptide comprises amino acids 44 to 59 of SEQ ID NO. 12, amino acids 75 to 81 of SEQ ID NO. 12, and amino acids 114 to 122 of SEQ ID NO. 12.

45. The isolated polypeptide of claim 39, wherein the isolated polypeptide comprises amino acids 44 to 55 of SEQ ID NO. 16, amino acids 71 to 77 of SEQ ID NO. 16, and amino acids 110 to 118 of SEQ ID NO. 16.

46. The isolated polypeptide of claim 39, wherein the isolated polypeptide comprises amino acids 46 to 56 of SEQ ID NO. 20, amino acids 72 to 78 of SEQ ID NO. 20, and amino acids 111 to 119 of SEQ ID NO. 20.

47. An isolated polynucleotide comprising a sequence encoding a polypeptide comprising at least one complementarity determining region (CDR) selected from CDR1a, CDR2a, or CDR3a
wherein CDR1a comprises the amino acid sequence a b c d e, wherein amino acid a is selected from asparagine, threonine, phenylalanine, or serine; amino acid b is selected from alanine or tyrosine; amino acid c is selected from tryptophan, tyrosine, or glycine; amino acid d is selected from methionine or tryptophan; and amino acid e is selected from serine, asparagine, or histidine;
wherein CDR2a comprises the amino acid sequence f g h i j k l m n o p q r s t, wherein amino acid f is selected from arginine or valine; amino acid g is isoleucine; amino acid h is selected from lysine, tyrosine, or tryptophan; amino acid i is selected from serine, isoleucine, tyrosine, threonine, or arginine; amino acid j is selected from lysine, serine, or aspartic acid; amino acid k is selected from threonine or glycine; amino acid l is selected from aspartic acid, serine, or glutamic acid; amino acid m is selected from glycine, threonine, or asparagine; amino acid n is selected from glycine, asparagine, lysine, or threonine; amino acid o is selected from threonine or tyrosine; amino acid p is selected from threonine, isoleucine, asparagine, or tyrosine; amino acid q is selected from aspartic acid, proline, or alanine; amino acid r is selected from tyrosine, serine, or aspartic acid; amino acid s is selected from glycine, alanine, leucine, or serine; and amino acid t is selected from alanine, lysine, or valine;
wherein CDR3a comprises the amino acid sequence u v w x y z a′ b′ c′ d′ e′ f′, wherein amino acid u is selected from aspartic acid, glycine, methionine, or serine; amino acid v is selected from arginine, glycine, aspartic acid, tyrosine, or phenylalanine; amino acid w is selected from tyrosine, valine, glycine, or leucine; amino acid x is selected from phenylalanine, aspartic acid, tyrosine, or tryptophan; amino acid y is selected from phenylalanine, aspartic acid, tyrosine, or isoleucine; amino acid z is selected from glycine, tyrosine, proline, valine, or phenylalanine; amino acid a′ is selected from glutamic acid, serine, tyrosine, tryptophan, or alanine; amino acid b′ is selected from phenylalanine, glycine, tyrosine, threonine, or serine; amino acid c′ is selected from proline, tyrosine, serine, lysine, or glycine; amino acid d′ is selected from phenylalanine, tyrosine, or glycine; amino acid e′ is selected from aspartic acid, tyrosine, arginine, or histidine; and amino acid f′ is selected from tyrosine, valine, glycine, arginine, or threonine; and
wherein the polypeptide, in association with an antibody light chain, is capable of binding OX40L.

48. The isolated polynucleotide of claim 47, wherein the sequence encoding a polypeptide is a sequence encoding an antibody heavy chain variable region.

49. The isolated polynucleotide of claim 47, wherein the isolated polynucleotide encodes a single-chain antibody.

50. The isolated polynucleotide of claim 48, further comprising a polynucleotide sequence encoding an antibody heavy chain constant region.

51. The isolated polynucleotide of claim 50, wherein the isolated polynucleotide comprises a sequence encoding a polypeptide comprising an amino acid sequence as set forth in SEQ ID NO. 2; SEQ ID NO. 6; SEQ ID NO. 10; SEQ ID NO. 14; SEQ ID NO. 18; or SEQ ID NO. 22.

52. The isolated polynucleotide of claim 50, wherein the isolated polynucleotide comprises a nucleotide sequence as set forth in SEQ ID NO. 1; SEQ ID NO. 5; SEQ ID NO. 9; SEQ ID NO. 13; SEQ ID NO. 17; or SEQ ID NO. 21.

53. An isolated polynucleotide comprising a sequence encoding a polypeptide comprising at least one complementarity determining region (CDR) selected from CDR1b, CDR2b, or CDR3b wherein CDR1b comprises the amino acid sequence a1 b1 c1 d1 e1 f1 g1 h1 i1 j1 k1, wherein amino acid a1 is arginine; amino acid b1 is selected from alanine or serine; amino acid c1 is serine; amino acid d1 is glutamine; amino acid e1 is selected from glycine or serine; amino acid f1 is selected from isoleucine, valine, or leucine; amino acid g1 is selected from serine or valine; amino acid h1 is selected from asparagine, serine, or histidine; amino acid i1 is selected from histidine, asparagine, serine, or tyrosine; amino acid j1 is selected from leucine, tyrosine, or aspartic acid; and amino acid k1 is selected from valine, leucine, glycine, or asparagine;
wherein CDR2b comprises the amino acid sequence l1 m1 n1 o1 p1 q1 r1, wherein amino acid l1 is selected from alanine, glycine, or lysine; amino acid m1 is selected from alanine or lysine; amino acid n1 is selected from serine or phenylalanine; amino acid o1 is selected from threonine, serine, or asparagine; amino acid p1 is selected from leucine or arginine; amino acid q1 is selected from glutamine, alanine, or phenylalanine; and amino acid r1 is selected from serine or threonine;
wherein CDR3b comprises the amino acid sequence s1 t1 u1 v1 w1 x1 y1 z1 a1′, wherein amino acid s1 is selected from glutamine or methionine; and amino acid t1 is selected from lysine or glutamine; amino acid u1 is selected from tyrosine, alanine, serine, or phenylalanine; amino acid v1 is selected from asparagine, glycine, threonine, or tyrosine; amino acid w1 is selected from serine, glycine, or glutamine; amino acid x1 is selected from alanine, serine, isoleucine, or threonine; amino acid y1 is selected from proline or leucine; amino acid z1 is selected from leucine, tryptophan, or phenylalanine; and amino acid a1′ is threonine; and
wherein the polypeptide, in association with an antibody heavy chain, is capable of binding OX40L.

54. The isolated polynucleotide of claim 53, wherein the sequence encoding a polypeptide is a sequence encoding an antibody light chain variable region.

55. The isolated polynucleotide of claim 53, wherein the isolated polynucleotide encodes a single-chain antibody.

56. The isolated polynucleotide of claim 54, further comprising a polynucleotide sequence encoding an antibody light chain constant region.

57. The isolated polynucleotide of claim 56, wherein the isolated polynucleotide comprises a sequence encoding a polypeptide comprising an amino acid sequence as set forth in SEQ ID NO. 4; SEQ ID NO. 8; SEQ ID NO. 12; SEQ ID NO. 16; or SEQ ID NO. 20.

58. The isolated polynucleotide of claim 56, wherein the isolated polynucleotide comprises a nucleotide sequence as set forth in SEQ ID NO. 3; SEQ ID NO. 7; SEQ ID NO. 11; SEQ ID NO. 15; or SEQ ID NO. 19.

59. An isolated anti-OX40L antibody, comprising a variable region and a constant region, wherein the antibody comprises:
(i) a first polypeptide comprising at least one complementarity determining region (CDR) selected from CDR1a, CDR2a, or CDR3a
wherein CDR1a comprises the amino acid sequence a b c d e, wherein amino acid a is selected from asparagine, threonine, phenylalanine, or serine; amino acid b is selected from alanine or tyrosine; amino acid c is selected from tryptophan, tyrosine, or glycine; amino acid d is selected from methionine or tryptophan; and amino acid e is selected from serine, asparagine, or histidine;
wherein CDR2a comprises the amino acid sequence f g h i j k l m n o p q r s t, wherein amino acid f is selected from arginine or valine; amino acid g is isoleucine; amino acid h is selected from lysine, tyrosine, or tryptophan; amino acid i is selected from serine, isoleucine, tyrosine, threonine, or arginine; amino acid j is selected from lysine, serine, or aspartic acid; amino acid k is selected from threonine or glycine; amino acid l is selected from aspartic acid, serine, or glutamic acid; amino acid m is selected from glycine, threonine, or asparagine; amino acid n is selected from glycine, asparagine, lysine, or threonine; amino acid o is selected from threonine or tyrosine; amino acid p is selected from threonine, isoleucine, asparagine, or tyrosine; amino acid q is selected from aspartic acid, proline, or alanine; amino acid r is selected from tyrosine, serine, or aspartic acid; amino acid s is selected from glycine, alanine, leucine, or serine; and amino acid t is selected from alanine, lysine, or valine;
wherein CDR3a comprises the amino acid sequence u v w x y z a′ b′ c′ d′ e′ f′, wherein amino acid u is selected from aspartic acid, glycine, methionine, or serine; amino acid v is selected from arginine, glycine, aspartic acid, tyrosine, or phenylalanine; amino acid w is selected from tyrosine, valine, glycine, or leucine; amino acid x is selected from phenylalanine, aspartic acid, tyrosine, or tryptophan; amino acid y is selected from phenylalanine, aspartic acid, tyrosine, or isoleucine; amino acid z is selected from glycine, tyrosine, proline, valine, or phenylalanine; amino acid a′ is selected from glutamic acid, serine, tyrosine, tryptophan, or alanine; amino acid b′ is selected from phenylalanine, glycine, tyrosine, threonine, or serine; amino acid c′ is selected from proline, tyrosine, serine, lysine, or glycine; amino acid d′ is selected from phenylalanine, tyrosine, or glycine; amino acid e′ is selected from aspartic acid, tyrosine, arginine, or histidine; and amino acid f′ is selected from tyrosine, valine, glycine, arginine, or threonine; and
wherein the first polypeptide, in association with an antibody light chain, is capable of binding OX40L; and
(ii) a second polypeptide comprising at least one complementarity determining region (CDR) selected from CDR1b, CDR2b, or CDR3b
wherein CDR1b comprises the amino acid sequence a1 b1 c1 d1 e1 f1 g1 h1 i1 j1 k1, wherein amino acid a1 is arginine; amino acid b1 is selected from alanine or serine; amino acid c1 is serine; amino acid d1 is glutamine; amino acid e1 is selected from glycine or serine; amino acid f1 is selected from isoleucine, valine, or leucine; amino acid g1 is selected from serine or valine; amino acid h1 is selected from asparagine, serine, or histidine; amino acid i1 is selected from histidine, asparagine, serine, or tyrosine; amino acid j1 is selected from leucine, tyrosine, or aspartic acid; and amino acid k1 is selected from valine, leucine, glycine, or asparagine;
wherein CDR2b comprises the amino acid sequence l1 m1 n1 o1 p1 q1 r1, wherein amino acid l1 is selected from alanine, glycine, or lysine; amino acid m1 is selected from alanine or lysine; amino acid n1 is selected from serine or phenylalanine; amino acid o1 is selected from threonine, serine, or asparagine; amino acid p1 is selected from leucine or arginine; amino acid q1 is selected from glutamine, alanine, or phenylalanine; and amino acid r1 is selected from serine or threonine;
wherein CDR3b comprises the amino acid sequence s1 t1 u1 v1 w1 x1 y1 z1 a1′, wherein amino acid s1 is selected from glutamine or methionine; and amino acid t1 is selected from lysine or glutamine; amino acid u1 is selected from tyrosine, alanine, serine, or phenylalanine; amino acid v1 is selected from asparagine, glycine, threonine, or tyrosine; amino acid w1 is selected from serine, glycine, or glutamine; amino acid x1 is selected from alanine, serine, isoleucine, or threonine; amino acid y1 is selected from proline or leucine; amino acid z1 is selected from leucine, tryptophan, or phenylalanine; and amino acid a1′ is threonine; and
wherein the second polypeptide, in association with an antibody heavy chain, is capable of binding OX40L.

60. The isolated antibody of claim 59, wherein the antibody is a human antibody.

61. The isolated antibody of claim 59, wherein the antibody is a chimeric antibody.

62. An isolated anti-OX40L antibody, comprising a variable region and a constant region, wherein the antibody comprises:
a first polypeptide comprising complementarity determining regions (CDRs) as set forth in SEQ ID NO. 2 and a second polypeptide comprising CDRs as set forth in SEQ ID NO. 4;
a first polypeptide comprising CDRs as set forth in SEQ ID NO. 6 and a second polypeptide comprising CDRs as set forth in SEQ ID NO. 8;
a first polypeptide comprising CDRs as set forth in SEQ ID NO. 10 and a second polypeptide comprising CDRs as set forth in SEQ ID NO. 12;
a first polypeptide comprising CDRs as set forth in SEQ ID NO. 14 and a second polypeptide comprising CDRs as set forth in SEQ ID NO. 16; or
a first polypeptide comprising CDRs as set forth in SEQ ID NO. 18 and a second polypeptide comprising CDRs as set forth in SEQ ID NO. 20.

63. A method for detecting the presence or absence of OX40L in a sample comprising:
a) combining the antibody of claim 59 and the sample;
b) separating antibodies bound to an antigen from unbound antibodies; and
c) detecting the presence or absence of antibodies bound to the antigen.

64. The method of claim 63, wherein the method uses an enzyme linked immunosorbent assay (ELISA).

65. A method for treating an inflammatory disease in a patient comprising administering a therapeutically effective amount of the antibody of claim 59 to the patient.

66. The method of claim 65, wherein the inflammatory disease is selected from at least one of rheumatoid arthritis, osteoarthritis, graft-versus-host disease, inflammatory bowel disease, Crohn's Disease, ulcerative colitis, multiple sclerosis, psoriasis, or proliferative lupus nephritis.

67. An expression vector comprising a polynucleotide of claim 47.

68. An expression vector comprising a polynucleotide of claim 53.

69. A cell comprising at least one of the expression vectors of claim 67 or claim 68.

70. A method of making a polypeptide comprising producing the polypeptide in a cell comprising the expression vector of claim 67 in conditions suitable to express the polynucleotide contained therein to produce the polypeptide.

71. A method of making a polypeptide comprising producing the polypeptide in a cell comprising the expression vector of claim 68 in conditions suitable to express the polynucleotide contained therein to produce the polypeptide.

72. A method of making an anti-OX40L antibody comprising producing the antibody in a cell comprising the expression vector of claim 67 and further comprising the expression vector of claim 68 in conditions suitable to express the polynucleotides contained therein to produce the antibody.

73. A pharmaceutical composition comprising the antibody of claim 59 and a pharmaceutically acceptable carrier.

74. An isolated antibody that specifically binds to an epitope that is specifically bound by at least one of Ab A, Ab B, Ab C, Ab D, Ab E, Ab F, Ab G, Ab H, Ab I, or Ab J.


SEQUENCE LISTING (26 sequences)

SEQ ID NO: 1 - DNA, 1416 bp, Homo sapiens
atggagtttgggctgagctggattttccttgctgctattttaaaaggtgtccagtgtgaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaatagactacgctgcacccgtgaaaggcagattcaccatctcaagagatgattcaaaaaacacgctgtatctgcaaatgaacagcctgaaaaccgaggacacagccgtgtattactgtaccacagatcggtatttcgatttttggagtggttattatacggtctttgactactggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - protein, 472 aa, Homo sapiens
MEFGLSWIFLAAILKGVQCEVQLVESGGGLVKPGGSLRLSCAASGFTFSNAWMSWVRQAPGKGLEWVGRIKSKTDGGTIDYAAPVKGRFTISRDDSKNTLYLQMNSLKTEDTAVYYCTTDRYFDFWSGYYTVFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - DNA, 708 bp, Homo sapiens
atggacatgagggtgcccgctcagctcctgggactcctgctgctctggctcccagataccagatgtgacatccagatgacccagtctccatcctccctgtctgcatctgtaggagacagagtcaccatcacttgccgggcgagtcagggcattagcaatcatttagtctggtatcagcagaaagtagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtttaacagtgccccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 4 - protein, 236 aa, Homo sapiens
MDMRVPAQLLGLLLLWLPDTRCDIQMTQSPSSLSASVGDRVTITCRASQGISNHLVWYQQKVGKVPKLLIYAASTLQSGVPSRFSGSGSGTDFTLTISSLQPEDVATYYCQKFNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1413 bp, Homo sapiens
atgaaacacctgtggttcttcctcctgctggtggcagctcccagatgggtcctgtcccaggtgcagctgcaggagtcgggcccaggactggtgaagccttcggagaccctgtccctcacctgcactgtctctggtggctccatcagtacttactactggaactggatccggcagcccgccgggaagggactggagtggattgggcgtatctatattagtgggagcaccaactacaacccctccctcaagagtcgagtcaccatgtcagtagacacgtccaagaaccagttctccctgaagctgagctctgtgaccgccgcggacacggccgtatattactgtgcgagagatagggtggatatagtggctacgaaggggcactactactacggtatggacgtctggggccaagggaccaccgtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 6 - protein, 471 aa, Homo sapiens
MKHLWFFLLLVAAPRWVLSQVQLQESGPGLVKPSETLSLTCTVSGGSISTYYWNWIRQPAGKGLEWIGRIYISGSTNYNPSLKSRVTMSVDTSKNQFSLKLSSVTAADTAVYYCARDRVDIVATKGHYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - DNA, 708 bp, Homo sapiens
atggacatgagggtgcccgctcagctcctggggctcctgctactctggctccgaggtgccagatgtgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctttgctgcatccagtttgcaaagtggggtcccatcaaggttcagtggcagtggatctgggacagatttcactctcaccatcagcagtctgcaacctgaagattttgcaacttactactgtcaacagagttacagtaccccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 8 - protein, 236 aa, Homo sapiens
MDMRVPAQLLGLLLLWLRGARCDIQMTQSPSSLSASVGDRVTITCRASQSISSYLNWYQQKPGKAPKLLIFAASSLQSGVPSRFSGSGSGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 1416 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtttctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagggggttatgattacgtttgggggagttatcgccgtaactccgactttgactactggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 10 - protein, 472 aa, Homo sapiens
MEFGLSWVFLVALLRGVQCQVQLVESGGGVVQPGRSLRLSCAASGFTFSFYGMHWVRQAPGKGLEWVAVIWYDGSNKYYADSVKGRFTISRDNSKNTLYLQMNSLRAEDTAVYYCARGGYDYVWGSYRRNSDFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - DNA, 717 bp, Homo sapiens
atgaggctccttgctcagcttctggggctgctaatgctctgggtccctggatccagtggggatattgtgatgacccagactccactctcctcacctgtcacccttggacagccggcctccatctcctgcaggtctagtcaaagcctcgtacacagtgatggaaacacctacttgagttggcttcagcagaggccaggccagcctccaagactcctaatttataagaaatttaaccggttctctggggtcccagacagattcagtggcagtggggcagggacagatttcacactgaaaatcagcagggtggaagctgaggatgtcggggtttattactgcatgcaagctacacaaattcctctcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 12 - protein, 239 aa, Homo sapiens
MRLLAQLLGLLMLWVPGSSGDIVMTQTPLSSPVTLGQPASISCRSSQSLVHSDGNTYLSWLQQRPGQPPRLLIYKKFNRFSGVPDRFSGSGAGTDFTLKISRVEAEDVGVYYCMQATQIPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1404 bp, Homo sapiens
atgaaacacctgtggttcttcctcctgctggtggcagctcccagatgggtcctgtcccaggtgcagctgcaggagtcgggcccaggactggtgaagccttcggagaccctgtccctcacctgcactgtctctggtggctccatcagtagttactactggaactggatccggcagcccgccgggaagggactggaatggattgggcgtatctataccagtgggagcaccacctacaacccctccctcaagagtcgagtcaccatgtcagtagacacgtccaagaaccagttctccctgaagctgagctctgtgaccgccgcggacacggccgtgtattactgtgcgagaatggacggtgactacccctactactactactacggtatggacgtctggggccaagggatcaccgtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - protein, 468 aa, Homo sapiens
MKHLWFFLLLVAAPRWVLSQVQLQESGPGLVKPSETLSLTCTVSGGSISSYYWNWIRQPAGKGLEWIGRIYTSGSTTYNPSLKSRVTMSVDTSKNQFSLKLSSVTAADTAVYYCARMDGDYPYYYYYGMDVWGQGITVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - DNA, 705 bp, Homo sapiens
atggaaaccccagcgcagcttctcttcctcctgctactctggctcccagatatcaccggagaaattgtgttgacgcagtctccaggcaccctgtctttgtctccaggggaaagagccaccctctcctgcagggccagtcagagtgttagcagcaactacttagcctggcaccagcagaaacctggccaggctcccaggctcctcatccatggtgcatccagcagggccactggcatcccagacaggttcagtggcagtgggtctgggacagacttcactctcaccatcagcagactggagcctgaagattttgcagtgtattactgtcagcagtatggtggctcactgtggacgttcggccaagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 16 - protein, 235 aa, Homo sapiens
METPAQLLFLLLLWLPDITGEIVLTQSPGTLSLSPGERATLSCRASQSVSSNYLAWHQQKPGQAPRLLIHGASSRATGIPDRFSGSGSGTDFTLTISRLEPEDFAVYYCQQYGGSLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 1410 bp, Homo sapiens
atggagtttgggctgagctggattttccttgctgctattttaaaaggtgtccagtgtgaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaaggaaaactgatggtgggacaatagactacgctgcacccgtgaaaggcagattcaccatctcaagagctgattcaaaaaacacgctgtttctgcaaatgaacagcctgataaccgaggacacagccgtgtattactgtaccatctcgtattactatgattatagtggttattactacgtggaccactggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 18 - protein, 470 aa, Homo sapiens
MEFGLSWIFLAAILKGVQCEVQLVESGGGLVKPGGSLRLSCAASGFTFSNAWMSWVRQAPGKGLEWVGRIKRKTDGGTIDYAAPVKGRFTISRADSKNTLFLQMNSLITEDTAVYYCTISYYYDYSGYYYVDHWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - DNA, 708 bp, Homo sapiens
atggacatgagggtgcccgctcagctcctgggactcctgctgctctggctcccagataccagatgtgacatccagatgacccagtctccatcctccctgtctgcatctgtaggagacagagtcaccatcacttgccgggcgagtcagggcattagcaatcatttagtctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 20 - protein, 236 aa, Homo sapiens
MDMRVPAQLLGLLLLWLPDTRCDIQMTQSPSSLSASVGDRVTITCRASQGISNHLVWYQQKPGKVPKLLIYAASTLQSGVPSRFSGSGSGTDFTLTISSLQPEDVATYYCQKYNSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 1404 bp, Homo sapiens
atggagtttgggctgagctggattttcctcgctgctattttaaaaggtgtccagtgtgaggtgcagctggtggagtctgggggaggcttggtaaagcctggggggtcccttagactctcctgtgcagcctctggattcactttcagtaacgcctggatgagctgggtccgccaggctccagggaaggggctggagtggattggccgtattaaaagcaaaactgaaggtgggacaacagactatggtgcacccgtgaaaggcagattcaccatctcaagagatgattcaaaaaacacgctgtatctgcaaatgaacagcctgaaaaccgaggacacagccgtgtattcctgtaccacagatttcctatggttcggggagttcccttttgactactggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 22 - protein, 468 aa, Homo sapiens
MEFGLSWIFLAAILKGVQCEVQLVESGGGLVKPGGSLRLSCAASGFTFSNAWMSWVRQAPGKGLEWIGRIKSKTEGGTTDYGAPVKGRFTISRDDSKNTLYLQMNSLKTEDTAVYSCTTDFLWFGEFPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 13 aa, Artificial Sequence
  Description of Artificial Sequence Synthetic peptide
RXSEXXXXXXXXT

SEQ ID NO: 24 - protein, 14 aa, Artificial Sequence
  Description of Artificial Sequence Synthetic peptide
RXSEXXXXXXXXTY

SEQ ID NO: 25 - protein, 15 aa, Artificial Sequence
  Description of Artificial Sequence Synthetic peptide
RXSEXXXXXXXXTYL

SEQ ID NO: 26 - protein, 16 aa, Artificial Sequence
  Description of Artificial Sequence Synthetic peptide
RXSEXXXXXXXXTYLS